(19) United States
(12) Patent Application Publication
(10) Pub. No.: US 2020/0308276 A1
(43) Pub. Date: Oct. 1, 2020

(54) ANTICANCER COMBINATION THERAPY

(71) Applicant: Boehringer Ingelheim International GmbH, Ingelheim am Rhein (DE)

(72) Inventors: Vittoria ZINZALLA, Vienna (AT); Markus Johann BAUER, Vienna (AT); Barbara DROBITS-HANDL, Vienna (AT)

(21) Appl. No.: 16/830,316

(22) Filed: Mar. 26, 2020

(30) Foreign Application Priority Data
Mar. 29, 2019 (EP) 19166375.6

(51) Int. Cl. C07K 16/28; A61P 35/00; A61K 9/00

(52) U.S. Cl. CPC C07K 16/28; A61P 35/00; A61K 2039/545; A61K 9/0019; C07K 2317/565; C07K 16/2818

(57) ABSTRACT

The invention describes anti-cancer therapies comprising using an polypeptide capable of specifically binding to LRP5 and LRP6 in combination with an anti-PD1 antibody, each as described herein.

### FIELD OF THE INVENTION

The present invention relates to a combination therapy in the treatment of cancer and to compounds for use in such a combination therapy. The compounds for combination are an LRP5/6 antagonist and a PD-1 antagonist.

### BACKGROUND OF THE INVENTION

Activation of the Wnt signaling pathway requires binding of extracellular Wnt ligands to the Frizzled receptor and to the co-receptor LRP5 (Accession number: UniProtKB-O75197/LRP5_HUMAN) or its closely related homologue LRP6 (Accession number: UniProtKB-O75581/LRP6_HUMAN). There are 19 Wnt proteins and 10 Frizzled receptors in mammalian cells. In the absence of Wnt ligand, cytoplasmic beta-catenin is phosphorylated by a protein complex consisting of the scaffolding proteins Axin and APC and the kinases GSK3beta and CK1a. Subsequent recognition by the ubiquitin ligase beta-TrcP leads to ubiquitin-mediated degradation of beta-catenin. In the presence of Wnt ligand, binding of Wnt to Frizzled and LRP5 or LRP6 leads to recruitment of the cytoplasmic effector protein Dvl and phosphorylation of the LRP5 or LRP6 cytoplasmic tail, which provides the docking site for Axin. Axin sequestration by LRP5 or LRP6 leads to the inactivation of the Axin-APC-GSK3beta complex and, therefore, intracellular beta-catenin stabilization and accumulation. Hence, cytoplasmic levels of beta-catenin rise, and beta-catenin migrates to the nucleus and complexes with members of the T-cell factor (TCF)/Lymphoid enhancer-binding factor (LEF) family of transcription factors. Basal transcription machinery and transcriptional co-activators are then recruited, including cAMP response element-binding protein (CREB)-binding protein (CBP) or its homolog p300, leading to expression of various target genes, including Axin2, cyclin D1, Naked1, Notum and c-Myc.

An additional level of ligand-dependent Wnt pathway regulation is mediated by the E3 ligase RNF43, and its closely related homologue ZNRF3, and by the secreted R-Spondin proteins (de Lau et al. “The R-spondin/Lgr5/Rnf43 module: regulator of Wnt signal strength”. Genes Dev. 2014; 28(4):305-16). RNF43 mediates the ubiquitination of the Frizzled/LRP5 or LRP6 receptor complex at the cell surface, leading to its degradation and, thereby, inhibiting ligand-dependent Wnt pathway activity. The activity of RNF43 is counteracted by the R spondin family members (R-spondin 1 to 4 ligands). When R-Spondin ligand is present, it removes RNF43 from the cell surface, allowing Frizzled/LRP5 or LRP6 complex accumulation and enhancement of Wnt signaling in the presence of Wnt ligands.

Hyperactivation of Wnt signaling is involved in the pathogenesis of various, albeit not all, types of cancer in at least two different ways: in some cancer types frequent mutations in downstream signaling molecules contribute to constitutively activated Wnt pathway (e.g. APC mutations in colorectal cancer; beta-catenin activating mutation in hepatocellular carcinoma), while in other types of cancer, such as e.g. Triple Negative Breast Cancer (TNBC), Non Small Cell Lung Cancer (NSCLC), pancreatic adenocarcinoma and in a subset of Colo-Rectal Cancer (CRC) and endometrial cancers, Wnt signaling activation is driven by a ligand dependent mechanism (i.e. by an autocrine/paracrine Wnt activation), as detected by beta-catenin intracellular accumulation. In NSCLC, TNBC and pancreatic adenocarcinoma, ligand dependent Wnt activation is mediated by multiple mechanisms, including increased expression of the Wnt ligands and/or of LRP5 and LRP6 receptors, or silencing of LRP5 and LRP6 negative regulator DKK1 (TNBC: Liu et al. “LRP6 overexpression defines a class of breast cancer subtype and is a target for therapy”. Proc Natl Acad Sci USA 2010; 107 (11):5136-41; Khramtsov et al. “Wnt/beta-catenin pathway activation is enriched in basal-like breast cancers and predicts poor outcome”. Am J Pathol. 2010; 176(6): 2911-20; NSCLC: Nakashima et al. “Wnt1 overexpression associated with tumor proliferation and a poor prognosis in non-small cell lung cancer patients”. Oncol Rep. 2008; 19(1):203-9; Pancreatic cancer: Zhang et al. “Canonical wnt signaling is required for pancreatic carcinogenesis”. Cancer Res. 2013; 73(15):4909-22). In particular, published data have shown that in healthy tissues (e.g. mammary and lung epithelium), beta-catenin is localized solely at the plasma membrane. In contrast, the majority of TNBC, NSCLC and pancreatic adenocarcinoma primary clinical samples showed beta-catenin intracellular accumulation (i.e. in the cytoplasm/nucleus; biomarker of Wnt signaling activation), due to aberrant Wnt signaling. Recent publications have shown that ligand dependent Wnt signaling activation is mediated by mutated/inactivated RNF43 (Giannakis et al. “RNF43 is frequently mutated in colorectal and endometrial cancers”. Nat Genet. 2014; 46(12):1264-6) or by activating R-Spondin fusion transcripts (encoding R-spondin2 or R-spondin3 proteins driven by constitutively active strong promoters; Seshagiri et al. “Recurrent R-spondin fusions in colon cancer”. Nature 2012; 488(7413):660-4) in a subset of CRC and endometrial cancers. Inactivating RNF43 mutations and R-Spondin fusion transcripts have both been shown to augment ligand dependent Wnt signaling in vitro by increasing the abundance of Frizzled on the cell surface. Ligand dependent Wnt activation in tumors was shown to drive tumor growth and resistance to chemotherapy or immunotherapy, and is linked to recurrence in pre-clinical models.

Because LRP5 and LRP6 function as gatekeeper of ligand dependent Wnt signaling activation, it may be considered as target to achieve complete blockade of the pathway mediated by all 19 Wnt ligands and 10 Frizzled receptors.

An alternative method to the above described approach of directly targeting the cancer/cancer cells is cancer immunotherapy. Cancer immunotherapy is a branch of oncology in which the immune system is used to treat cancer, which is in stark contrast to existing common methods of treatment in which the tumour is directly excised or treated. This therapeutic concept is based on the identification of a number of proteins on the surface of T-cells which act to inhibit the immune function of these cells. Listed among these proteins is PD-1 (Programmed cell Death 1).

PD-1 is a cell surface receptor protein expressed on T-cells. PD-1 has two ligands, PD-L1 and PD-L2, which interact with the cell surface receptor. On ligand-binding, PD-1 induces an intracellular signal which negatively regulates T-cell response. Thus, typically, the protein functions as an “immune checkpoint” inhibitor, i.e. it acts to modulate the activity of cells in the immune system so as to regulate and limit autoimmune diseases. It has been recently understood that many cancers can protect themselves from the immune system by modifying “immune checkpoint” inhibitors and thus avoid detection.

Accordingly, it has also been shown in a range of different cancer settings that antagonistic PD-1 antibody molecules, such as e.g. nivolumab and pembrolizumab, can be used to stimulate the immune system and thereby treat cancer.

Despite the above described advances in the treatment of cancer, there is still a need for new therapeutic concepts for the treatment of cancer which show advantages over standard therapies. These advantages may include in vivo efficacy (e.g. improved clinical response, extend of the response, increase of the rate of response, duration of response, disease stabilization rate, duration of stabilization, time to disease progression, progression free survival (PFS) and/or overall survival (OS), later occurrence of resistance and the like), safe and well tolerated administration and reduced frequency and severity of adverse events. Specifically, there is a need for additional treatment options for patients with cancers like, e.g., lung cancer (e.g. NSCLC), melanoma, bladder and gastrointestinal cancers.

It is thus an object of the present invention to provide a novel treatment for cancer that is advantageous over treatments/methods of treatment currently used and/or known in the prior art.

### BRIEF SUMMARY OF THE INVENTION

The present invention provides a method for treating a patient with a hyperproliferative disease with an LRP5/LRP6 antagonist (this term is used interchangeably herein with the terms “polypeptide specifically binding to LRP5 and LRP6” or “polypeptide capable of specifically binding to LRP5 and LRP6”), together with an antibody specific for Programmed Cell Death 1 (PD-1) (this term is used interchangeably herein with the terms “anti PD-1 antibody”, “PD-1 antibody” or “PD-1 antagonist”), thereby antagonizing the PD-1 signaling pathway. Accordingly, the present invention provides a combination therapy comprising an LRP5/LRP6 antagonist and an anti-PD-1 antibody.

In one aspect, the invention provides a polypeptide capable of specifically binding to LRP5 and LRP6 for use in a method of treating and/or preventing a hyperproliferative disease, preferably cancer, wherein said method comprises that the polypeptide capable of specifically binding to LRP5 and LRP6 is to be administered in combination with a PD-1 antibody to a patient in need thereof, wherein the polypeptide capable of specifically binding to LRP5 and LRP6 is selected from the group consisting of
(i) a polypeptide comprising a first immunoglobulin single variable domain (ISVD) (a) comprising the following CDR sequences:
   CDR1: TYTVG (=SEQ ID NO:40)
   CDR2: AIRRRGSSTYYADSVKG (=SEQ ID NO:41)
   CDR3: DTRTVALLQYRYDY (=SEQ ID NO:42), and
   a second ISVD (b) comprising the following CDR sequences:
   CDR1: SYAMG (=SEQ ID NO:49)
   CDR2: AISWSGGSTYYADSVKG (=SEQ ID NO:50)
   CDR3: SPIPYGSLLRRRNNYDY (=SEQ ID NO:51);
(ii) a polypeptide comprising a first ISVD (a) comprising the following CDR sequences:
   CDR1: SYAMG (=SEQ ID NO:43)
   CDR2: AIRRSGRRTYYADSVKG (=SEQ ID NO:44)
   CDR3: ARRVRSSTRYNTGTWWWEY (=SEQ ID NO:45), and
   a second ISVD (b) comprising the following CDR sequences:
   CDR1: SYAMG (=SEQ ID NO:49)
   CDR2: AISWSGGSTYYADSVKG (=SEQ ID NO:50)
   CDR3: SPIPYGSLLRRRNNYDY (=SEQ ID NO:51);
(iii) a polypeptide comprising a first ISVD (a) comprising the following CDR sequences:
   CDR1: RYTMG (=SEQ ID NO:46)
   CDR2: AIVRSGGSTYYADSVKG (=SEQ ID NO:47)
   CDR3: DRRGRGENYILLYSSGRYEY (=SEQ ID NO:48), and
   a second ISVD (b) comprising the following CDR sequences:
   CDR1: SYAMG (=SEQ ID NO:49)
   CDR2: AISWSGGSTYYADSVKG (=SEQ ID NO:50)
   CDR3: SPIPYGSLLRRRNNYDY (=SEQ ID NO:51);
(iv) a polypeptide comprising a first ISVD (a) comprising the following CDR sequences:
   CDR1: TYTVG (=SEQ ID NO:40)
   CDR2: AIRRRGSSTYYADSVKG (=SEQ ID NO:41)
   CDR3: DTRTVALLQYRYDY (=SEQ ID NO:42), and
   a second ISVD (b) comprising the following CDR sequences:
   CDR1: SYAMG (=SEQ ID NO:52)
   CDR2: AISWRSGSTYYADSVKG (=SEQ ID NO:53)
   CDR3: DPRGYGVAYVSAYYEY (=SEQ ID NO:54);
(v) a polypeptide comprising a first ISVD (a) comprising the following CDR sequences:
   CDR1: SYAMG (=SEQ ID NO:43)
   CDR2: AIRRSGRRTYYADSVKG (=SEQ ID NO:44)
   CDR3: ARRVRSSTRYNTGTWWWEY (=SEQ ID NO:45), and
   a second ISVD (b) comprising the following CDR sequences:
   CDR1: SYAMG (=SEQ ID NO:52)
   CDR2: AISWRSGSTYYADSVKG (=SEQ ID NO:53)
   CDR3: DPRGYGVAYVSAYYEY (=SEQ ID NO:54);

and
(vi) a polypeptide comprising a first ISVD (a) comprising the following CDR sequences:
   CDR1: RYTMG (=SEQ ID NO:46)
   CDR2: AIVRSGGSTYYADSVKG (=SEQ ID NO:47)
   CDR3: DRRGRGENYILLYSSGRYEY (=SEQ ID NO:48), and
   a second ISVD (b) comprising the following CDR sequences:
   CDR1: SYAMG (=SEQ ID NO:52)
   CDR2: AISWRSGSTYYADSVKG (=SEQ ID NO:53)
   CDR3: DPRGYGVAYVSAYYEY (=SEQ ID NO:54);

and wherein the PD-1 antibody is selected from the group consisting of
(i) an anti-PD1 antibody comprising heavy chain CDRs comprising the amino acid sequence of SEQ ID NO:1 (HCDR1), SEQ ID NO:2 (HCDR2) and SEQ ID NO:3 (HCDR3) and light chain CDRs comprising the amino acid sequence of SEQ ID NO:4 (LCDR1), SEQ ID NO:5 (LCDR2) and SEQ ID NO:6 (LCDR3);
(ii) an anti-PD1 antibody comprising heavy chain CDRs comprising the amino acid sequence of SEQ ID NO:7 (HCDR1), SEQ ID NO:8 (HCDR2) and SEQ ID NO:9 (HCDR3) and light chain CDRs comprising the amino acid sequence of SEQ ID NO:10 (LCDR1), SEQ ID NO:11 (LCDR2) and SEQ ID NO:12 (LCDR3);

and
(iii) an anti-PD1 antibody comprising heavy chain CDRs comprising the amino acid sequence of SEQ ID NO:13 (HCDR1), SEQ ID NO:14 (HCDR2) and SEQ ID NO:15 (HCDR3) and light chain CDRs comprising the amino acid sequence of SEQ ID NO:16 (LCDR1), SEQ ID NO:17 (LCDR2) and SEQ ID NO:18 (LCDR3).

In another aspect, the invention provides a method of treating and/or preventing a hyperproliferative disease, preferably cancer, comprising administering to a patient in need thereof a therapeutically effective amount of a polypeptide capable of specifically binding to LRP5 and LRP6 and a therapeutically effective amount of a PD-1 antibody, wherein the polypeptide capable of specifically binding to LRP5 and LRP6 is selected from the group consisting of
(i) a polypeptide comprising a first immunoglobulin single variable domain (ISVD) (a) comprising the following CDR sequences:
   CDR1: TYTVG (=SEQ ID NO:40)
   CDR2: AIRRRGSSTYYADSVKG (=SEQ ID NO:41)
   CDR3: DTRTVALLQYRYDY (=SEQ ID NO:42), and
   a second ISVD (b) comprising the following CDR sequences:
   CDR1: SYAMG (=SEQ ID NO:49)
   CDR2: AISWSGGSTYYADSVKG (=SEQ ID NO:50)
   CDR3: SPIPYGSLLRRRNNYDY (=SEQ ID NO:51);
(ii) a polypeptide comprising a first ISVD (a) comprising the following CDR sequences:
   CDR1: SYAMG (=SEQ ID NO:43)
   CDR2: AIRRSGRRTYYADSVKG (=SEQ ID NO:44)
   CDR3: ARRVRSSTRYNTGTWWWEY (=SEQ ID NO:45), and
   a second ISVD (b) comprising the following CDR sequences:
   CDR1: SYAMG (=SEQ ID NO:49)
   CDR2: AISWSGGSTYYADSVKG (=SEQ ID NO:50)
   CDR3: SPIPYGSLLRRRNNYDY (=SEQ ID NO:51);
(iii) a polypeptide comprising a first ISVD (a) comprising the following CDR sequences:
   CDR1: RYTMG (=SEQ ID NO:46)
   CDR2: AIVRSGGSTYYADSVKG (=SEQ ID NO:47)
   CDR3: DRRGRGENYILLYSSGRYEY (=SEQ ID NO:48), and
   a second ISVD (b) comprising the following CDR sequences:
   CDR1: SYAMG (=SEQ ID NO:49)
   CDR2: AISWSGGSTYYADSVKG (=SEQ ID NO:50)
   CDR3: SPIPYGSLLRRRNNYDY (=SEQ ID NO:51);
(iv) a polypeptide comprising a first ISVD (a) comprising the following CDR sequences:
   CDR1: TYTVG (=SEQ ID NO:40)
   CDR2: AIRRRGSSTYYADSVKG (=SEQ ID NO:41)
   CDR3: DTRTVALLQYRYDY (=SEQ ID NO:42), and
   a second ISVD (b) comprising the following CDR sequences:
   CDR1: SYAMG (=SEQ ID NO:52)
   CDR2: AISWRSGSTYYADSVKG (=SEQ ID NO:53)
   CDR3: DPRGYGVAYVSAYYEY (=SEQ ID NO:54);
(v) a polypeptide comprising a first ISVD (a) comprising the following CDR sequences:
   CDR1: SYAMG (=SEQ ID NO:43)
   CDR2: AIRRSGRRTYYADSVKG (=SEQ ID NO:44)
   CDR3: ARRVRSSTRYNTGTWWWEY (=SEQ ID NO:45), and
   a second ISVD (b) comprising the following CDR sequences:
   CDR1: SYAMG (=SEQ ID NO:52)
   CDR2: AISWRSGSTYYADSVKG (=SEQ ID NO:53)
   CDR3: DPRGYGVAYVSAYYEY (=SEQ ID NO:54);

and
(vi) a polypeptide comprising a first ISVD (a) comprising the following CDR sequences:
CDR1: RYTMG (=SEQ ID NO:46)
CDR2: AIVRSGGSTYYADSVKG (=SEQ ID NO:47)
CDR3: DRRGRGENYILLYSSGRYEY (=SEQ ID NO:48), and
a second ISVD (b) comprising the following CDR sequences:
CDR1: SYAMG (=SEQ ID NO:52)
CDR2: AISWRSGSTYYADSVKG (=SEQ ID NO:53)
CDR3: DPRGYGVAYVSAYYEY (=SEQ ID NO:54);

and wherein the PD-1 antibody is selected from the group consisting of
(i) an anti-PD1 antibody comprising heavy chain CDRs comprising the amino acid sequence of SEQ ID NO:1 (HCDR1), SEQ ID NO:2 (HCDR2) and SEQ ID NO:3 (HCDR3) and light chain CDRs comprising the amino acid sequence of SEQ ID NO:4 (LCDR1), SEQ ID NO:5 (LCDR2) and SEQ ID NO:6 (LCDR3);
(ii) an anti-PD1 antibody comprising heavy chain CDRs comprising the amino acid sequence of SEQ ID NO:7 (HCDR1), SEQ ID NO:8 (HCDR2) and SEQ ID NO:9 (HCDR3) and light chain CDRs comprising the amino acid sequence of SEQ ID NO:10 (LCDR1), SEQ ID NO:11 (LCDR2) and SEQ ID NO:12 (LCDR3);

and
(iii) an anti-PD1 antibody comprising heavy chain CDRs comprising the amino acid sequence of SEQ ID NO:13 (HCDR1), SEQ ID NO:14 (HCDR2) and SEQ ID NO:15 (HCDR3) and light chain CDRs comprising the amino acid sequence of SEQ ID NO:16 (LCDR1), SEQ ID NO:17 (LCDR2) and SEQ ID NO:18 (LCDR3).

In another aspect, the invention provides a PD-1 antibody for use in a method of treating and/or preventing a hyperproliferative disease, preferably cancer, the method comprising that a PD-1 antibody is to be administered in combination with polypeptide capable of specifically binding to LRP5 and LRP6 to a patient in need thereof, wherein the polypeptide capable of specifically binding to LRP5 and LRP6 is selected from the group consisting of
(i) a polypeptide comprising a first immunoglobulin single variable domain (ISVD) (a) comprising the following CDR sequences:
   CDR1: TYTVG (=SEQ ID NO:40)
   CDR2: AIRRRGSSTYYADSVKG (=SEQ ID NO:41)
   CDR3: DTRTVALLQYRYDY (=SEQ ID NO:42), and
   a second ISVD (b) comprising the following CDR sequences:
   CDR1: SYAMG (=SEQ ID NO:49)
   CDR2: AISWSGGSTYYADSVKG (=SEQ ID NO:50)
   CDR3: SPIPYGSLLRRRNNYDY (=SEQ ID NO:51);
(ii) a polypeptide comprising a first ISVD (a) comprising the following CDR sequences:
   CDR1: SYAMG (=SEQ ID NO:43)
   CDR2: AIRRSGRRTYYADSVKG (=SEQ ID NO:44)
   CDR3: ARRVRSSTRYNTGTWWWEY (=SEQ ID NO:45), and
   a second ISVD (b) comprising the following CDR sequences:
   CDR1: SYAMG (=SEQ ID NO:49)
   CDR2: AISWSGGSTYYADSVKG (=SEQ ID NO:50)
   CDR3: SPIPYGSLLRRRNNYDY (=SEQ ID NO:51);
(iii) a polypeptide comprising a first ISVD (a) comprising the following CDR sequences:
   CDR1: RYTMG (=SEQ ID NO:46)
   CDR2: AIVRSGGSTYYADSVKG (=SEQ ID NO:47)
   CDR3: DRRGRGENYILLYSSGRYEY (=SEQ ID NO:48), and
   a second ISVD (b) comprising the following CDR sequences:
   CDR1: SYAMG (=SEQ ID NO:49)
   CDR2: AISWSGGSTYYADSVKG (=SEQ ID NO:50)
   CDR3: SPIPYGSLLRRRNNYDY (=SEQ ID NO:51);
(iv) a polypeptide comprising a first ISVD (a) comprising the following CDR sequences:
   CDR1: TYTVG (=SEQ ID NO:40)
   CDR2: AIRRRGSSTYYADSVKG (=SEQ ID NO:41)
   CDR3: DTRTVALLQYRYDY (=SEQ ID NO:42), and
   a second ISVD (b) comprising the following CDR sequences:
   CDR1: SYAMG (=SEQ ID NO:52)
   CDR2: AISWRSGSTYYADSVKG (=SEQ ID NO:53)
   CDR3: DPRGYGVAYVSAYYEY (=SEQ ID NO:54);
(v) a polypeptide comprising a first ISVD (a) comprising the following CDR sequences:
   CDR1: SYAMG (=SEQ ID NO:43)
   CDR2: AIRRSGRRTYYADSVKG (=SEQ ID NO:44)
   CDR3: ARRVRSSTRYNTGTWWWEY (=SEQ ID NO:45), and
   a second ISVD (b) comprising the following CDR sequences:
   CDR1: SYAMG (=SEQ ID NO:52)
   CDR2: AISWRSGSTYYADSVKG (=SEQ ID NO:53)
   CDR3: DPRGYGVAYVSAYYEY (=SEQ ID NO:54);

and
(vi) a polypeptide comprising a first ISVD (a) comprising the following CDR sequences:
   CDR1: RYTMG (=SEQ ID NO:46)
   CDR2: AIVRSGGSTYYADSVKG (=SEQ ID NO:47)
   CDR3: DRRGRGENYILLYSSGRYEY (=SEQ ID NO:48), and
   a second ISVD (b) comprising the following CDR sequences:
   CDR1: SYAMG (=SEQ ID NO:52)
   CDR2: AISWRSGSTYYADSVKG (=SEQ ID NO:53)
   CDR3: DPRGYGVAYVSAYYEY (=SEQ ID NO:54);

and wherein the PD-1 antibody is selected from the group consisting of
(i) an anti-PD1 antibody comprising heavy chain CDRs comprising the amino acid sequence of SEQ ID NO:1 (HCDR1), SEQ ID NO:2 (HCDR2) and SEQ ID NO:3 (HCDR3) and light chain CDRs comprising the amino acid sequence of SEQ ID NO:4 (LCDR1), SEQ ID NO:5 (LCDR2) and SEQ ID NO:6 (LCDR3);
(ii) an anti-PD1 antibody comprising heavy chain CDRs comprising the amino acid sequence of SEQ ID NO:7 (HCDR1), SEQ ID NO:8 (HCDR2) and SEQ ID NO:9 (HCDR3) and light chain CDRs comprising the amino acid sequence of SEQ ID NO:10 (LCDR1), SEQ ID NO:11 (LCDR2) and SEQ ID NO:12 (LCDR3);

and
(iii) an anti-PD1 antibody comprising heavy chain CDRs comprising the amino acid sequence of SEQ ID NO:13 (HCDR1), SEQ ID NO:14 (HCDR2) and SEQ ID NO:15 (HCDR3) and light chain CDRs comprising the amino acid sequence of SEQ ID NO:16 (LCDR1), SEQ ID NO:17 (LCDR2) and SEQ ID NO:18 (LCDR3).

In another aspect, the invention provides for the use of polypeptide capable of specifically binding to LRP5 and LRP6 for preparing a pharmaceutical composition for use in a method of treating and/or preventing a hyperproliferative disease, preferably cancer, wherein the polypeptide capable of specifically binding to LRP5 and LRP6 is to be used in combination with a PD-1 antibody, wherein the polypeptide capable of specifically binding to LRP5 and LRP6 is selected from the group consisting of
(i) a polypeptide comprising a first immunoglobulin single variable domain (ISVD) (a) comprising the following CDR sequences:
   CDR1: TYTVG (=SEQ ID NO:40)
   CDR2: AIRRRGSSTYYADSVKG (=SEQ ID NO:41)
   CDR3: DTRTVALLQYRYDY (=SEQ ID NO:42), and
   a second ISVD (b) comprising the following CDR sequences:
   CDR1: SYAMG (=SEQ ID NO:49)
   CDR2: AISWSGGSTYYADSVKG (=SEQ ID NO:50)
   CDR3: SPIPYGSLLRRRNNYDY (=SEQ ID NO:51);
(ii) a polypeptide comprising a first ISVD (a) comprising the following CDR sequences:
   CDR1: SYAMG (=SEQ ID NO:43)
   CDR2: AIRRSGRRTYYADSVKG (=SEQ ID NO:44)
   CDR3: ARRVRSSTRYNTGTWWWEY (=SEQ ID NO:45), and
   a second ISVD (b) comprising the following CDR sequences:
   CDR1: SYAMG (=SEQ ID NO:49)
   CDR2: AISWSGGSTYYADSVKG (=SEQ ID NO:50)
   CDR3: SPIPYGSLLRRRNNYDY (=SEQ ID NO:51);
(iii) a polypeptide comprising a first ISVD (a) comprising the following CDR sequences:
   CDR1: RYTMG (=SEQ ID NO:46)
   CDR2: AIVRSGGSTYYADSVKG (=SEQ ID NO:47)
   CDR3: DRRGRGENYILLYSSGRYEY (=SEQ ID NO:48), and
   a second ISVD (b) comprising the following CDR sequences:
   CDR1: SYAMG (=SEQ ID NO:49)
   CDR2: AISWSGGSTYYADSVKG (=SEQ ID NO:50)
   CDR3: SPIPYGSLLRRRNNYDY (=SEQ ID NO:51);
(iv) a polypeptide comprising a first ISVD (a) comprising the following CDR sequences:
   CDR1: TYTVG (=SEQ ID NO:40)
   CDR2: AIRRRGSSTYYADSVKG (=SEQ ID NO:41)
   CDR3: DTRTVALLQYRYDY (=SEQ ID NO:42), and
   a second ISVD (b) comprising the following CDR sequences:
   CDR1: SYAMG (=SEQ ID NO:52)
   CDR2: AISWRSGSTYYADSVKG (=SEQ ID NO:53)
   CDR3: DPRGYGVAYVSAYYEY (=SEQ ID NO:54);
(v) a polypeptide comprising a first ISVD (a) comprising the following CDR sequences:
   CDR1: SYAMG (=SEQ ID NO:43)
   CDR2: AIRRSGRRTYYADSVKG (=SEQ ID NO:44)
   CDR3: ARRVRSSTRYNTGTWWWEY (=SEQ ID NO:45), and
   a second ISVD (b) comprising the following CDR sequences:
   CDR1: SYAMG (=SEQ ID NO:52)
   CDR2: AISWRSGSTYYADSVKG (=SEQ ID NO:53)
   CDR3: DPRGYGVAYVSAYYEY (=SEQ ID NO:54);

and
(vi) a polypeptide comprising a first ISVD (a) comprising the following CDR sequences:
   CDR1: RYTMG (=SEQ ID NO:46)
   CDR2: AIVRSGGSTYYADSVKG (=SEQ ID NO:47)
   CDR3: DRRGRGENYILLYSSGRYEY (=SEQ ID NO:48), and
   a second ISVD (b) comprising the following CDR sequences:
   CDR1: SYAMG (=SEQ ID NO:52)
   CDR2: AISWRSGSTYYADSVKG (=SEQ ID NO:53)
   CDR3: DPRGYGVAYVSAYYEY (=SEQ ID NO:54);

and wherein the PD-1 antibody is selected from the group consisting of
(i) an anti-PD1 antibody comprising heavy chain CDRs comprising the amino acid sequence of SEQ ID NO:1 (HCDR1), SEQ ID NO:2 (HCDR2) and SEQ ID NO:3 (HCDR3) and light chain CDRs comprising the amino acid sequence of SEQ ID NO:4 (LCDR1), SEQ ID NO:5 (LCDR2) and SEQ ID NO:6 (LCDR3);
(ii) an anti-PD1 antibody comprising heavy chain CDRs comprising the amino acid sequence of SEQ ID NO:7 (HCDR1), SEQ ID NO:8 (HCDR2) and SEQ ID NO:9 (HCDR3) and light chain CDRs comprising the amino acid sequence of SEQ ID NO:10 (LCDR1), SEQ ID NO:11 (LCDR2) and SEQ ID NO:12 (LCDR3);

and
(iii) an anti-PD1 antibody comprising heavy chain CDRs comprising the amino acid sequence of SEQ ID NO:13 (HCDR1), SEQ ID NO:14 (HCDR2) and SEQ ID NO:15 (HCDR3) and light chain CDRs comprising the amino acid sequence of SEQ ID NO:16 (LCDR1), SEQ ID NO:17 (LCDR2) and SEQ ID NO:18 (LCDR3).

In another aspect, the invention provides for the use of a PD-1 antibody for preparing a pharmaceutical composition for use in a method of treating and/or preventing a hyperproliferative disease, preferably cancer, wherein the PD-1-antibody is to be used in combination with a polypeptide capable of specifically binding to LRP5 and LRP6 is selected from the group consisting of
(i) a polypeptide comprising a first immunoglobulin single variable domain (ISVD) (a) comprising the following CDR sequences:
   CDR1: TYTVG (=SEQ ID NO:40)
   CDR2: AIRRRGSSTYYADSVKG (=SEQ ID NO:41)
   CDR3: DTRTVALLQYRYDY (=SEQ ID NO:42), and
   a second ISVD (b) comprising the following CDR sequences:
   CDR1: SYAMG (=SEQ ID NO:49)
   CDR2: AISWSGGSTYYADSVKG (=SEQ ID NO:50)
   CDR3: SPIPYGSLLRRRNNYDY (=SEQ ID NO:51);
(ii) a polypeptide comprising a first ISVD (a) comprising the following CDR sequences:
   CDR1: SYAMG (=SEQ ID NO:43)
   CDR2: AIRRSGRRTYYADSVKG (=SEQ ID NO:44)
   CDR3: ARRVRSSTRYNTGTWWWEY (=SEQ ID NO:45), and
   a second ISVD (b) comprising the following CDR sequences:
   CDR1: SYAMG (=SEQ ID NO:49)
   CDR2: AISWSGGSTYYADSVKG (=SEQ ID NO:50)
   CDR3: SPIPYGSLLRRRNNYDY (=SEQ ID NO:51);
(iii) a polypeptide comprising a first ISVD (a) comprising the following CDR sequences:
   CDR1: RYTMG (=SEQ ID NO:46)
   CDR2: AIVRSGGSTYYADSVKG (=SEQ ID NO:47)
   CDR3: DRRGRGENYILLYSSGRYEY (=SEQ ID NO:48), and
   a second ISVD (b) comprising the following CDR sequences:
   CDR1: SYAMG (=SEQ ID NO:49)
   CDR2: AISWSGGSTYYADSVKG (=SEQ ID NO:50)
   CDR3: SPIPYGSLLRRRNNYDY (=SEQ ID NO:51);
(iv) a polypeptide comprising a first ISVD (a) comprising the following CDR sequences:
   CDR1: TYTVG (=SEQ ID NO:40)
   CDR2: AIRRRGSSTYYADSVKG (=SEQ ID NO:41)
   CDR3: DTRTVALLQYRYDY (=SEQ ID NO:42), and
   a second ISVD (b) comprising the following CDR sequences:
   CDR1: SYAMG (=SEQ ID NO:52)
   CDR2: AISWRSGSTYYADSVKG (=SEQ ID NO:53)
   CDR3: DPRGYGVAYVSAYYEY (=SEQ ID NO:54);
(v) a polypeptide comprising a first ISVD (a) comprising the following CDR sequences:
   CDR1: SYAMG (=SEQ ID NO:43)
   CDR2: AIRRSGRRTYYADSVKG (=SEQ ID NO:44)
   CDR3: ARRVRSSTRYNTGTWWWEY (=SEQ ID NO:45), and
   a second ISVD (b) comprising the following CDR sequences:
   CDR1: SYAMG (=SEQ ID NO:52)
   CDR2: AISWRSGSTYYADSVKG (=SEQ ID NO:53)
   CDR3: DPRGYGVAYVSAYYEY (=SEQ ID NO:54);

and
(vi) a polypeptide comprising a first ISVD (a) comprising the following CDR sequences:
   CDR1: RYTMG (=SEQ ID NO:46)
   CDR2: AIVRSGGSTYYADSVKG (=SEQ ID NO:47)
   CDR3: DRRGRGENYILLYSSGRYEY (=SEQ ID NO:48), and
   a second ISVD (b) comprising the following CDR sequences:
   CDR1: SYAMG (=SEQ ID NO:52)
   CDR2: AISWRSGSTYYADSVKG (=SEQ ID NO:53)
   CDR3: DPRGYGVAYVSAYYEY (=SEQ ID NO:54);

and wherein the PD-1 antibody is selected from the group consisting of
(i) an anti-PD1 antibody comprising heavy chain CDRs comprising the amino acid sequence of SEQ ID NO:1 (HCDR1), SEQ ID NO:2 (HCDR2) and SEQ ID NO:3 (HCDR3) and light chain CDRs comprising the amino acid sequence of SEQ ID NO:4 (LCDR1), SEQ ID NO:5 (LCDR2) and SEQ ID NO:6 (LCDR3);
(ii) an anti-PD1 antibody comprising heavy chain CDRs comprising the amino acid sequence of SEQ ID NO:7 (HCDR1), SEQ ID NO:8 (HCDR2) and SEQ ID NO:9 (HCDR3) and light chain CDRs comprising the amino acid sequence of SEQ ID NO:10 (LCDR1), SEQ ID NO:11 (LCDR2) and SEQ ID NO:12 (LCDR3);

and
(iii) an anti-PD1 antibody comprising heavy chain CDRs comprising the amino acid sequence of SEQ ID NO:13 (HCDR1), SEQ ID NO:14 (HCDR2) and SEQ ID NO:15 (HCDR3) and light chain CDRs comprising the amino acid sequence of SEQ ID NO:16 (LCDR1), SEQ ID NO:17 (LCDR2) and SEQ ID NO:18 (LCDR3).

In another aspect, the invention provides for a pharmaceutical composition comprising:
a polypeptide capable of specifically binding to LRP5 and LRP6;
a PD-1 antibody; and,
optionally, one or more pharmaceutically acceptable carriers, excipients and/or vehicles; wherein the polypeptide capable of specifically binding to LRP5 and LRP6 is selected from the group consisting of
(i) a polypeptide comprising a first immunoglobulin single variable domain (ISVD) (a) comprising the following CDR sequences:
   CDR1: TYTVG (=SEQ ID NO:40)
   CDR2: AIRRRGSSTYYADSVKG (=SEQ ID NO:41)
   CDR3: DTRTVALLQYRYDY (=SEQ ID NO:42), and
   a second ISVD (b) comprising the following CDR sequences:
   CDR1: SYAMG (=SEQ ID NO:49)
   CDR2: AISWSGGSTYYADSVKG (=SEQ ID NO:50)
   CDR3: SPIPYGSLLRRRNNYDY (=SEQ ID NO:51);
(ii) a polypeptide comprising a first ISVD (a) comprising the following CDR sequences:
   CDR1: SYAMG (=SEQ ID NO:43)
   CDR2: AIRRSGRRTYYADSVKG (=SEQ ID NO:44)
   CDR3: ARRVRSSTRYNTGTWWWEY (=SEQ ID NO:45), and
   a second ISVD (b) comprising the following CDR sequences:
   CDR1: SYAMG (=SEQ ID NO:49)
   CDR2: AISWSGGSTYYADSVKG (=SEQ ID NO:50)
   CDR3: SPIPYGSLLRRRNNYDY (=SEQ ID NO:51);
(iii) a polypeptide comprising a first ISVD (a) comprising the following CDR sequences:
   CDR1: RYTMG (=SEQ ID NO:46)
   CDR2: AIVRSGGSTYYADSVKG (=SEQ ID NO:47)
   CDR3: DRRGRGENYILLYSSGRYEY (=SEQ ID NO:48), and
   a second ISVD (b) comprising the following CDR sequences:
   CDR1: SYAMG (=SEQ ID NO:49)
   CDR2: AISWSGGSTYYADSVKG (=SEQ ID NO:50)
   CDR3: SPIPYGSLLRRRNNYDY (=SEQ ID NO:51);
(iv) a polypeptide comprising a first ISVD (a) comprising the following CDR sequences:
   CDR1: TYTVG (=SEQ ID NO:40)
   CDR2: AIRRRGSSTYYADSVKG (=SEQ ID NO:41)
   CDR3: DTRTVALLQYRYDY (=SEQ ID NO:42), and
   a second ISVD (b) comprising the following CDR sequences:
   CDR1: SYAMG (=SEQ ID NO:52)
   CDR2: AISWRSGSTYYADSVKG (=SEQ ID NO:53)
   CDR3: DPRGYGVAYVSAYYEY (=SEQ ID NO:54);
(v) a polypeptide comprising a first ISVD (a) comprising the following CDR sequences:
   CDR1: SYAMG (=SEQ ID NO:43)
   CDR2: AIRRSGRRTYYADSVKG (=SEQ ID NO:44)
   CDR3: ARRVRSSTRYNTGTWWWEY (=SEQ ID NO:45), and
   a second ISVD (b) comprising the following CDR sequences:
   CDR1: SYAMG (=SEQ ID NO:52)
   CDR2: AISWRSGSTYYADSVKG (=SEQ ID NO:53)
   CDR3: DPRGYGVAYVSAYYEY (=SEQ ID NO:54);

and
(vi) a polypeptide comprising a first ISVD (a) comprising the following CDR sequences:
   CDR1: RYTMG (=SEQ ID NO:46)
   CDR2: AIVRSGGSTYYADSVKG (=SEQ ID NO:47)
   CDR3: DRRGRGENYILLYSSGRYEY (=SEQ ID NO:48), and
   a second ISVD (b) comprising the following CDR sequences:
   CDR1: SYAMG (=SEQ ID NO:52)
   CDR2: AISWRSGSTYYADSVKG (=SEQ ID NO:53)
   CDR3: DPRGYGVAYVSAYYEY (=SEQ ID NO:54);

and wherein the PD-1 antibody is selected from the group consisting of
(i) an anti-PD1 antibody comprising heavy chain CDRs comprising the amino acid sequence of SEQ ID NO:1 (HCDR1), SEQ ID NO:2 (HCDR2) and SEQ ID NO:3 (HCDR3) and light chain CDRs comprising the amino acid sequence of SEQ ID NO:4 (LCDR1), SEQ ID NO:5 (LCDR2) and SEQ ID NO:6 (LCDR3);
(ii) an anti-PD1 antibody comprising heavy chain CDRs comprising the amino acid sequence of SEQ ID NO:7 (HCDR1), SEQ ID NO:8 (HCDR2) and SEQ ID NO:9 (HCDR3) and light chain CDRs comprising the amino acid sequence of SEQ ID NO:10 (LCDR1), SEQ ID NO:11 (LCDR2) and SEQ ID NO:12 (LCDR3);

and
(iii) an anti-PD1 antibody comprising heavy chain CDRs comprising the amino acid sequence of SEQ ID NO:13 (HCDR1), SEQ ID NO:14 (HCDR2) and SEQ ID NO:15 (HCDR3) and light chain CDRs comprising the amino acid sequence of SEQ ID NO:16 (LCDR1), SEQ ID NO:17 (LCDR2) and SEQ ID NO:18 (LCDR3).

In some embodiments, the pharmaceutical composition is for use in a method of treating and/or preventing a hyperproliferative disease, preferably cancer.

In another aspect, the invention provides for a kit comprising in one or more containers
a first pharmaceutical composition or dosage form comprising a polypeptide capable of specifically binding to LRP5 and LRP6 and, optionally, one or more pharmaceutically acceptable carriers, excipients and/or vehicles;
a second pharmaceutical composition or dosage form comprising a PD-1 antibody and, optionally, one or more pharmaceutically acceptable carriers, excipients and/or vehicles; and
optionally a package insert comprising printed instructions;

wherein the polypeptide capable of specifically binding to LRP5 and LRP6 is selected from the group consisting of
(i) a polypeptide comprising a first immunoglobulin single variable domain (ISVD) (a) comprising the following CDR sequences:
   CDR1: TYTVG (=SEQ ID NO:40)
   CDR2: AIRRRGSSTYYADSVKG (=SEQ ID NO:41)
   CDR3: DTRTVALLQYRYDY (=SEQ ID NO:42), and
   a second ISVD (b) comprising the following CDR sequences:
   CDR1: SYAMG (=SEQ ID NO:49)
   CDR2: AISWSGGSTYYADSVKG (=SEQ ID NO:50)
   CDR3: SPIPYGSLLRRRNNYDY (=SEQ ID NO:51);
(ii) a polypeptide comprising a first ISVD (a) comprising the following CDR sequences:
   CDR1: SYAMG (=SEQ ID NO:43)
   CDR2: AIRRSGRRTYYADSVKG (=SEQ ID NO:44)
   CDR3: ARRVRSSTRYNTGTWWWEY (=SEQ ID NO:45), and
   a second ISVD (b) comprising the following CDR sequences:
   CDR1: SYAMG (=SEQ ID NO:49)
   CDR2: AISWSGGSTYYADSVKG (=SEQ ID NO:50)
   CDR3: SPIPYGSLLRRRNNYDY (=SEQ ID NO:51);
(iii) a polypeptide comprising a first ISVD (a) comprising the following CDR sequences:
   CDR1: RYTMG (=SEQ ID NO:46)
   CDR2: AIVRSGGSTYYADSVKG (=SEQ ID NO:47)
   CDR3: DRRGRGENYILLYSSGRYEY (=SEQ ID NO:48), and
   a second ISVD (b) comprising the following CDR sequences:
   CDR1: SYAMG (=SEQ ID NO:49)
   CDR2: AISWSGGSTYYADSVKG (=SEQ ID NO:50)
   CDR3: SPIPYGSLLRRRNNYDY (=SEQ ID NO:51);
(iv) a polypeptide comprising a first ISVD (a) comprising the following CDR sequences:
   CDR1: TYTVG (=SEQ ID NO:40)
   CDR2: AIRRRGSSTYYADSVKG (=SEQ ID NO:41)
   CDR3: DTRTVALLQYRYDY (=SEQ ID NO:42), and
   a second ISVD (b) comprising the following CDR sequences:
   CDR1: SYAMG (=SEQ ID NO:52)
   CDR2: AISWRSGSTYYADSVKG (=SEQ ID NO:53)
   CDR3: DPRGYGVAYVSAYYEY (=SEQ ID NO:54);
(v) a polypeptide comprising a first ISVD (a) comprising the following CDR sequences:
   CDR1: SYAMG (=SEQ ID NO:43)
   CDR2: AIRRSGRRTYYADSVKG (=SEQ ID NO:44)
   CDR3: ARRVRSSTRYNTGTWWWEY (=SEQ ID NO:45), and
   a second ISVD (b) comprising the following CDR sequences:
   CDR1: SYAMG (=SEQ ID NO:52)
   CDR2: AISWRSGSTYYADSVKG (=SEQ ID NO:53)
   CDR3: DPRGYGVAYVSAYYEY (=SEQ ID NO:54);

and
(vi) a polypeptide comprising a first ISVD (a) comprising the following CDR sequences:
   CDR1: RYTMG (=SEQ ID NO:46)
   CDR2: AIVRSGGSTYYADSVKG (=SEQ ID NO:47)
   CDR3: DRRGRGENYILLYSSGRYEY (=SEQ ID NO:48), and
   a second ISVD (b) comprising the following CDR sequences:
   CDR1: SYAMG (=SEQ ID NO:52)
   CDR2: AISWRSGSTYYADSVKG (=SEQ ID NO:53)
   CDR3: DPRGYGVAYVSAYYEY (=SEQ ID NO:54);

and wherein the PD-1 antibody is selected from the group consisting of
(i) an anti-PD1 antibody comprising heavy chain CDRs comprising the amino acid sequence of SEQ ID NO:1 (HCDR1), SEQ ID NO:2 (HCDR2) and SEQ ID NO:3 (HCDR3) and light chain CDRs comprising the amino acid sequence of SEQ ID NO:4 (LCDR1), SEQ ID NO:5 (LCDR2) and SEQ ID NO:6 (LCDR3);
(ii) an anti-PD1 antibody comprising heavy chain CDRs comprising the amino acid sequence of SEQ ID NO:7 (HCDR1), SEQ ID NO:8 (HCDR2) and SEQ ID NO:9 (HCDR3) and light chain CDRs comprising the amino acid sequence of SEQ ID NO:10 (LCDR1), SEQ ID NO:11 (LCDR2) and SEQ ID NO:12 (LCDR3);

and
(iii) an anti-PD1 antibody comprising heavy chain CDRs comprising the amino acid sequence of SEQ ID NO:13 (HCDR1), SEQ ID NO:14 (HCDR2) and SEQ ID NO:15 (HCDR3) and light chain CDRs comprising the amino acid sequence of SEQ ID NO:16 (LCDR1), SEQ ID NO:17 (LCDR2) and SEQ ID NO:18 (LCDR3).

In some embodiments, the kit according to the invention is for use in a method of treating and/or preventing a hyperproliferative disease, preferably cancer.

In preferred embodiments of the invention the polypeptide capable of specifically binding to LRP5 and LRP6 is selected from the group consisting of
(i) a polypeptide comprising a first ISVD comprising an amino acid sequence of SEQ ID NO:58, and a second ISVD comprising the sequence of SEQ ID NO:61;
(ii) a polypeptide comprising a first ISVG comprising an amino acid sequence of SEQ ID NO:59 and a second ISVD comprising the sequence of SEQ ID NO:61;
(iii) a polypeptide comprising a first ISVD comprising the sequence of SEQ ID NO:60, and a second ISVD comprising the sequence of SEQ ID NO:61;
(iv) a polypeptide comprising a first ISVD comprising an amino acid sequence of SEQ ID NO:58 and a second ISVD comprising the sequence of SEQ ID NO:62;
(v) a polypeptide comprising a first ISVD comprising an amino acid sequence of SEQ ID NO:59 and a second ISVD comprising the sequence of SEQ ID NO:62; and
(vi) a polypeptide comprising a first ISVD comprising an amino acid sequence of SEQ ID NO:60 and a second ISVD comprising the sequence of SEQ ID NO:62;
preferably wherein the polypeptide capable of specifically binding to LRP5 and LRP6 further comprises an Alb11 domain comprising the amino acid sequence of SEQ ID NO:63.

In particularly preferred embodiments, the polypeptide capable of specifically binding to LRP5 and LRP6 comprises a polypeptide comprising an amino acid sequence selected from the group consisting of SEQ ID NO: 64, SEQ ID NO:65 and SEQ ID NO:66.

In preferred embodiments of the invention, the anti-PD1 antibody is selected from the group consisting of
(i) an antibody having a heavy chain variable domain comprising the amino acid sequence of SEQ ID NO:19 and a light chain variable domain comprising the amino acid sequence of SEQ ID NO:20;
(ii) an antibody having a heavy chain variable domain comprising the amino acid sequence of SEQ ID NO:21 and a light chain variable domain comprising the amino acid sequence of SEQ ID NO:22;
(iii) an antibody having a heavy chain variable domain comprising the amino acid sequence of SEQ ID NO:23 and a light chain variable domain comprising the amino acid sequence of SEQ ID NO:24;
(iv) an antibody having a heavy chain variable domain comprising the amino acid sequence of SEQ ID NO:25 and a light chain variable domain comprising the amino acid sequence of SEQ ID NO:26; and
(v) an antibody having a heavy chain variable domain comprising the amino acid sequence of SEQ ID NO:27 and a light chain variable domain comprising the amino acid sequence of SEQ ID NO:28.

In particularly preferred embodiments of the invention, the PD-1 antibody is selected from the group consisting of
(i) an antibody comprising a heavy chain comprising the amino acid sequence of SEQ ID NO: 29 and a light chain comprising the amino acid sequence of SEQ ID NO: 30;
(ii) an antibody comprising a heavy chain comprising the amino acid sequence of SEQ ID NO: 31 and a light chain comprising the amino acid sequence of SEQ ID NO: 32;
(iii) an antibody comprising a heavy chain comprising the amino acid sequence of SEQ ID NO: 33 and a light chain comprising the amino acid sequence of SEQ ID NO: 34;
(iv) an antibody comprising a heavy chain comprising the amino acid sequence of SEQ ID NO: 35 and a light chain comprising the amino acid sequence of SEQ ID NO: 36; and
(v) an antibody comprising a heavy chain comprising the amino acid sequence of SEQ ID NO: 37 and a light chain comprising the amino acid sequence of SEQ ID NO: 38.

In some embodiments of the invention, the PD-1 antibody is to be administered simultaneously, concurrently, sequentially, successively, alternately or separately with the polypeptide capable of specifically binding to LRP5 and LRP6.

In preferred embodiments, the polypeptide capable of specifically binding to LRP5 and LRP6 and the PD-1 antibody are to be administered according to the following treatment regimen:
(i) a first treatment period, wherein the polypeptide capable of specifically binding to LRP5 and LRP6 and the PD-1 antibody are to be administered simultaneously or concurrently, preferably every three or four weeks; and
(ii) a second treatment period, wherein only the PD-1 antibody is to be administered and the polypeptide capable of specifically binding to LRP5 and LRP6 is not to be administered, preferably wherein the PD-1 antibody is to be administered every three or four weeks.

In preferred embodiments of the invention, the hyperproliferative disease to be treated is a cancer selected from the group consisting of gastrointestinal cancers, melanoma tumours, bladder cancer and lung cancer (e.g. NSCLC), even more preferably the cancer is an immunotherapy-resistant gastrointestinal cancer (including but not limited to esophageal cancer (e.g., gastroesophageal junction cancer), stomach (gastric) cancer, hepatocellularcarcinoma, biliary tract cancer (e.g., cholangiocarcinoma), gallbladder cancer, pancreatic cancer or colorectal cancer (CRC)), immunotherapy-resistant melanoma, immunotherapy-resistant bladder cancer or an immunotherapy-resistant lung cancer.

In alternative preferred embodiments of the invention, the hyperproliferative disease to be treated is a solid immunotherapy-resistant tumour.

### BRIEF DESCRIPTION OF THE FIGURES

FIG. 1A-1H: shows the anti-tumor activity of the exemplary LRP5/LRP6 antagonist as single agent and in combination with an exemplary antibody to PD-1, in a subcutaneous syngeneic mouse model derived from the breast cancer cell line EMT6 in Balb/c mice. FIG. 1A: Measurement of tumor volume at the indicated days after treatment with isotype matched antibody; 1B: with LRP5/6 antagonist; 1C: with PD-1 antibody; and 1D: with LRP5/6 antagonist+PD-1 antibodies. FIG. 1E: Measurement of tumor shrinkage response at the indicated days after treatment with isotype matched antibody; 1F: with LRP5/6 antagonist; 1G: with PD-1 antibody; and 1H: with LRP5/6 antagonist+PD-1 antibodies. The numbering indicated with * shows the number of mice out of the total investigated mice in which a response was observed, i.e. in which the ratio between the tumor volume at the end and the start of treatment is below 1 (i.e. indicating shrinkage of the tumor).

FIG. 2 shows tumor-infiltrating CD8+ lymphocytes (% of positive cells in the total area of tumor) assessed by immunohistochemical staining of tumor samples at day 16 of the exemplary LRP5/LRP6 antagonist as single agent and in combination with an exemplary antibody to PD-1, in a subcutaneous syngeneic mouse model derived from the breast cancer cell line EMT6 in Balb/c mice.

FIGS. 3A and 3B: FIG. 3A: shows that Wnt signaling activation blocks PBMC mediated inhibition of cancer cell viability, which is restored by treatment with an LRP5/LRP6 antagonist.

FIG. 3B: demonstrates that a combination of the LRP5/LRP6 antagonist and an anti-human PD-1 antibody leads to the enhancement of PBMC-mediated tumor cell killing, when compared to monotherapy with the LRP5/LRP6 antagonist, in an in vitro co-culture assay with tumor cells (NCI-H1437 non-small cell lung cancer cell line) and human PBMC. NCI-H1437 cells were stably transfected to express a red fluorescent protein (mKate2) and cultured in 3D as spheroids. Wnt3a (1 μg/ml), LRP5/LRP6 antagonist (LRP5/6; 1000 nM), anti-human PD-1 antibody (PD1; 200 nM) and an isotype control of the anti-PD-1 antibody (iso; 200 nM) were added at 0 hour. Activated human PBMCs (pre-treatment with anti-CD3/CD28 agonists for 72 hours) were added to the tumor cells at 0 hour. Tumor cell viability was measured as fluorescent signal (mKate2 RFU) at the indicated time points (days).

### DETAILED DESCRIPTION OF THE INVENTION

### Definitions

The above and other aspects and embodiments of the invention will become clear from the further description herein, in which:

Unless indicated or defined otherwise, all terms used have their usual meaning in the art, which will be clear to the person skilled in the art to which this invention belongs. In case of conflict, the patent specification, including definitions, will prevail. Reference is for example made to the standard handbooks, such as Sambrook et al, “Molecular Cloning: A Laboratory Manual” (2nd Ed.), Vols. 1-3, Cold Spring Harbor Laboratory Press (1989); Lewin, “Genes IV”, Oxford University Press, New York, (1990), and Roitt et al., “Immunology” (2^{nd} Ed.), Gower Medical Publishing, London, New York (1989), as well as to the general background art cited herein. Furthermore, unless indicated otherwise, all methods, steps, techniques and manipulations that are not specifically described in detail can be performed and have been performed in a manner known per se, as will be clear to the skilled person. Reference is for example again made to the standard handbooks, to the general background art referred to above and to the further references cited therein.

The term “antibody” encompasses antibodies, antibody fragments, antibody-like molecules and conjugates with any of the above. Antibodies include, but are not limited to, poly- or monoclonal, chimeric, humanized, human, mono-, bi- or multispecific antibodies. The term “antibody” shall encompass complete immunoglobulins as they are produced by lymphocytes and for example present in blood sera, monoclonal antibodies secreted by hybridoma cell lines, polypeptides produced by recombinant expression in host cells, which have the binding specificity of immunoglobulins or monoclonal antibodies, and molecules which have been derived from such immunoglobulins, monoclonal antibodies, or polypeptides by further processing while retaining their binding specificity. In particular, the term “antibody” includes complete immunoglobulins comprising two heavy chains and two light chains. In another embodiment, the term encompasses a fragment of an immunoglobulin, like Fab fragments. In another embodiment, the term “antibody” encompasses a polypeptide having one or more variable domains derived from an immunoglobulin, like single chain antibodies (scFv), single domain antibodies, and the like.

A “human antibody” is one which possesses an amino acid sequence which corresponds to that of an antibody produced by a human cell or derived from a non-human source that utilizes human antibody repertoires or other human antibody-encoding sequences. This definition of a human antibody specifically excludes a humanized antibody comprising non-human antigen-binding residues.

The term “recombinant human antibody”, as used herein, is intended to include all human antibodies that are prepared, expressed, created or isolated by recombinant means, such as antibodies isolated from a host cell such as a NSO or CHO cell or from an animal (e.g. a mouse) that is transgenic for human immunoglobulin genes or antibodies expressed using a recombinant expression vector transfected into a host cell. Such recombinant human antibodies have variable and constant regions in a rearranged form. The recombinant human antibodies according to the invention have been subjected to in vivo somatic hypermutation. Thus, the amino acid sequences of the VH and VL regions of the recombinant antibodies are sequences that, while derived from and related to human germ line VH and VL sequences, may not naturally exist within the human antibody germ line repertoire in vivo.

A “humanized” antibody refers to a chimeric antibody comprising amino acid residues from non-human hypervariable regions (HVRs) and amino acid residues from human framework regions (FRs). In certain embodiments, a humanized antibody will comprise substantially all of at least one, and typically two, variable domains, in which all or substantially all of the HVRs (e.g. complementary determining regions (CDRs)) correspond to those of a non-human antibody, and all or substantially the entire framework regions (FRs) correspond to those of a human antibody. A humanized antibody optionally may comprise at least a portion of an antibody constant region derived from a human antibody. A “humanized form” of an antibody, e.g. a non-human antibody, refers to an antibody that has undergone humanization.

The expressions “variable domain” or “variable region” or Fv as used herein denotes each of the pair of light and heavy chains which is involved directly in binding the antibody to the antigen. The variable domain of a light chain is abbreviated as “VL” and the variable domain of a heavy chain is abbreviated as “VH”. The variable light and heavy chain domains have the same general structure and each domain comprises four framework (FR) regions whose sequences are widely conserved, connected by three HVRs (or CDRs). The framework regions adopt a beta-sheet conformation and the CDRs may form loops connecting the beta-sheet structure. The CDRs in each chain are held in their three-dimensional structure by the framework regions and form together with the CDRs from the other chain the antigen binding site. The antibody's heavy and light chain CDR regions play a particularly important role in the binding specificity/affinity of the antibodies according to the invention and therefore provide a further object of the invention.

Within the context of this invention, reference to CDR's in connection with antibodies (e.g. PD1 antibodies) is based on the definition of Chothia (Chothia and Lesk, J. Mol. Biol. 1987, 196: 901-917), together with Kabat (E. A. Kabat, T. T. Wu, H. Bilofsky, M. Reid-Miller and H. Perry, Sequence of Proteins of Immunological Interest, National Institutes of Health, Bethesda (1983)).

Unless indicated otherwise, the terms “immunoglobulin” and “immunoglobulin sequence”—whether used herein to refer to a heavy chain antibody or to a conventional 4-chain antibody—are used as general terms to include both the full-size antibody, the individual chains thereof, as well as all parts, domains or fragments thereof (including but not limited to antigen-binding domains or fragments such as VHH domains or VH/VL domains, respectively). In addition, the term “sequence” as used herein (for example in terms like “immunoglobulin sequence”, “antibody sequence”, “(single) variable domain sequence”, “VHH sequence” or “protein sequence”), should generally be understood to include both the relevant amino acid sequence as well as nucleic acid sequences or nucleotide sequences encoding the same, unless the context requires a more limited interpretation.

The term “domain” (of a polypeptide or protein) as used herein refers to a folded protein structure which has the ability to retain its tertiary structure independently of the rest of the protein. Generally, domains are responsible for discrete functional properties of proteins, and in many cases can be added, removed or transferred to other proteins without loss of function of the remainder of the protein and/or of the domain.

The term “immunoglobulin domain” as used herein refers to a globular region of an antibody chain (such as e.g. a chain of a conventional 4-chain antibody or of a heavy chain antibody), or to a polypeptide that essentially consists of such a globular region. Immunoglobulin domains are characterized in that they retain the immunoglobulin fold characteristic of antibody molecules, which consists of a 2-layer sandwich of about 7 antiparallel beta-strands arranged in two beta-sheets, optionally stabilized by a conserved disulphide bond.

The term “immunoglobulin variable domain” as used herein means an immunoglobulin domain essentially consisting of four “framework regions” which are referred to in the art and herein as “framework region 1” or “FR1”; as “framework region 2” or “FR2”; as “framework region 3” or “FR3”; and as “framework region 4” or “FR4”, respectively; which framework regions are interrupted by three “complementarity determining regions” or “CDRs”, which are referred to in the art and herein as “complementarity determining region 1” or “CDR1”; as “complementarity determining region 2” or “CDR2”; and as “complementarity determining region 3” or “CDR3”, respectively. Thus, the general structure or sequence of an immunoglobulin variable domain can be indicated as follows: FR1-CDR1-FR2-CDR2-FR3-CDR3-FR4. It is the immunoglobulin variable domain(s) that confer specificity to an antibody for the antigen by carrying the antigen-binding site.

The term “immunoglobulin single variable domain” (or ISVD) as used herein means an immunoglobulin variable domain which is capable of specifically binding to an epitope of the antigen without pairing with an additional variable immunoglobulin domain. One example of ISVDs in the meaning of the present invention are “domain antibodies”, such as the ISVDs VH and VL (VH domains and VL domains). Another important example of ISVDs are “VHH domains” (or simply “VHHs”) from camelids, as defined hereinafter.

In view of the above definition, the antigen-binding domain of a conventional 4-chain antibody (such as an IgG, IgM, IgA, IgD or IgE molecule; known in the art) or of a Fab fragment, a F(ab′)2 fragment, an Fv fragment such as a disulphide linked Fv or a scFv fragment, or a diabody (all known in the art) derived from such conventional 4-chain antibody, would normally not be regarded as an ISVD, as, in these cases, binding to the respective epitope of an antigen would normally not occur by one (single) immunoglobulin domain but by a pair of (associating) immunoglobulin domains such as light and heavy chain variable domains, i.e. by a VH-VL pair of immunoglobulin domains, which jointly bind to an epitope of the respective antigen.

“VHH domains”, also known as VHHs, V_{H}H domains, VHH antibody fragments, and VHH antibodies, have originally been described as the antigen binding immunoglobulin (variable) domain of “heavy chain antibodies” (i.e. of “antibodies devoid of light chains”; Hamers-Casterman C, Atarhouch T, Muyldermans S, Robinson G, Hamers C, Songa E B, Bendahman N, Hamers R.: “Naturally occurring antibodies devoid of light chains”; Nature 363, 446-448 (1993)). The term “VHH domain” has been chosen in order to distinguish these variable domains from the heavy chain variable domains that are present in conventional 4-chain antibodies (which are referred to herein as “V_{H} domains” or “VH domains”) and from the light chain variable domains that are present in conventional 4-chain antibodies (which are referred to herein as “V_{L} domains” or “VL domains”). VHH domains can specifically bind to an epitope without an additional antigen binding domain (as opposed to VH or VL domains in a conventional 4-chain antibody, in which case the epitope is recognized by a VL domain together with a VH domain). VHH domains are small, robust and efficient antigen recognition units formed by a single immunoglobulin domain.

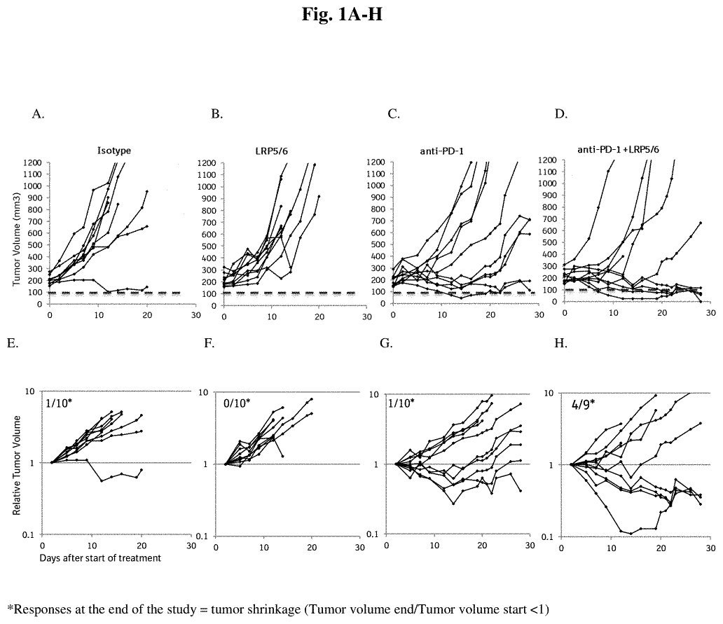

In the context of the present invention, the terms VHH domain, VHH, V_{H}H domain, VHH antibody fragment, VHH antibody, as well as “Nanobody®” and “Nanobody® domain” (“Nanobody” being a trademark of the company Ablynx N.V.; Ghent; Belgium) are used interchangeably and are representatives of ISVDs (having the structure: FR1-CDR1-FR2-CDR2-FR3-CDR3-FR4 and specifically binding to an epitope without requiring the presence of a second immunoglobulin variable domain), and which can also be distinguished from VH domains by the so-called “hallmark residues”, as defined in e.g. WO2009/109635, FIG. 1.

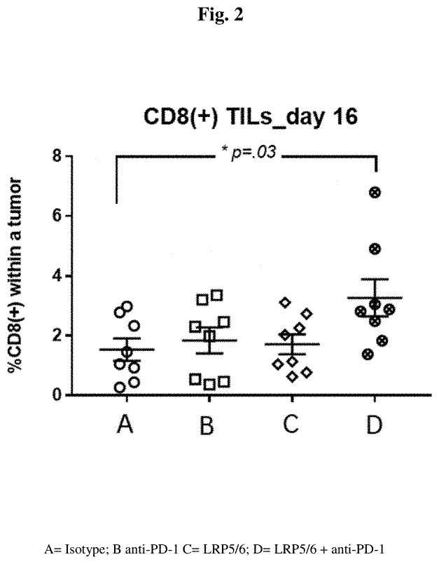

The amino acid residues of a VHH domain are numbered according to the general numbering for V_{H} domains given by Kabat et al. (“Sequence of proteins of immunological interest”, US Public Health Services, NIH Bethesda, Md., Publication No. 91), as applied to VHH domains from Camelids, as shown e.g. in FIG. 2 of Riechmann and Muyldermans, J. Immunol.

Methods 231, 25-38 (1999). According to this numbering,
FR1 comprises the amino acid residues at positions 1-30,
CDR1 comprises the amino acid residues at positions 31-35,
FR2 comprises the amino acids at positions 36-49,
CDR2 comprises the amino acid residues at positions 50-65,
FR3 comprises the amino acid residues at positions 66-94,
CDR3 comprises the amino acid residues at positions 95-102, and
FR4 comprises the amino acid residues at positions 103-113.

However, it should be noted that—as is well known in the art for V_{H} domains and for VHH domains—the total number of amino acid residues in each of the CDRs may vary and, consequently, may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that although the numbering of the amino acid residues of a VHH domain is based on the numbering according to Kabat, the actual numbering of the amino acid residues in the actual sequence can differ. As this kind of variation is well known in the art, the respective numbering and the allocation of framework regions and CDRs within such a sequence can be determined by the skilled person without further ado.

Alternative methods for numbering the amino acid residues of V_{H} domains, which methods can also be applied in an analogous manner to VHH domains, are known in the art. However, in the present description, claims and figures in connection with ISVDs described herein, the numbering according to Kabat and applied to VHH domains as described above will be followed, unless indicated otherwise.

The total number of amino acid residues in a VHH domain will usually be in the range of from 110 to 120, often between 112 and 115. It should however be noted that smaller and longer sequences may also be suitable for the purposes described herein.

Methods of obtaining VHH domains binding to a specific antigen or epitope have been described earlier, e.g. in WO2006/040153 and WO2006/122786. VHH domains derived from camelids can be “humanized” by replacing one or more amino acid residues in the amino acid sequence of the original VHH sequence by one or more of the amino acid residues that occur at the corresponding position(s) in a VH domain from a conventional 4-chain antibody from a human being. A humanized VHH domain can contain one or more fully human framework region sequences, and, in an even more specific embodiment, can contain human framework region sequences derived from DP-29, DP-47, DP-51, or parts thereof, optionally combined with JH sequences, such as JH5.

The terms “epitope” and “antigenic determinant”, which can be used interchangeably, refer to the part of a macromolecule, such as a polypeptide, that is recognized by antigen-binding molecules, such as conventional antibodies or the polypeptides of the invention, and more particularly by the antigen-binding site of said molecules. Epitopes define the minimum binding site for an immunoglobulin, and thus represent the target of specificity of an immunoglobulin.

The part of an antigen-binding molecule (such as a conventional antibody or a polypeptide described herein) that recognizes the epitope is called a paratope.

The term “biparatopic” (antigen-)binding molecule or “biparatopic” polypeptide as used herein shall mean a polypeptide comprising a first ISVD and a second ISVD as herein defined, wherein these two variable domains are capable of binding to two different epitopes of one antigen, which epitopes are not normally bound at the same time by one monospecific immunoglobulin, such as e.g. a conventional antibody or one ISVD. The biparatopic polypeptides according to the invention are composed of variable domains which have different epitope specificities, and do not contain mutually complementary variable domain pairs which bind to the same epitope. They do therefore not compete with each other for binding to LRP5 or LRP6.

A polypeptide (such as an immunoglobulin, an antibody, an ISVD, or generally an antigen binding molecule or a fragment thereof) that can “bind”, “bind to”, “specifically bind”, is “capable of specifically binding to”, or “specifically bind to”, that “has affinity for” and/or that “has specificity for” a certain epitope, antigen or protein (or for at least one part, fragment or epitope thereof) is said to be “against” or “directed against” said epitope, antigen or protein or is a “binding” molecule with respect to such epitope, antigen or protein.

Generally, the term “specificity” refers to the number of different types of antigens or epitopes to which a particular antigen-binding molecule or antigen-binding protein (such as an immunoglobulin, an antibody, an ISVD) can bind. The specificity of an antigen-binding protein can be determined based on its affinity and/or avidity. The affinity, represented by the equilibrium constant for the dissociation of an antigen with an antigen-binding protein (K_{D}), is a measure for the binding strength between an epitope and an antigen-binding site on the antigen-binding protein: the lesser the value of the K_{D}, the stronger the binding strength between an epitope and the antigen-binding molecule (alternatively, the affinity can also be expressed as the affinity constant (K_{A}), which is 1/K_{D}). As will be clear to the skilled person (for example on the basis of the further disclosure herein), affinity can be determined in a manner known per se, depending on the specific antigen of interest. Avidity is the measure of the strength of binding between an antigen-binding molecule (such as an immunoglobulin, an antibody, an ISVD) and the pertinent antigen. Avidity is related to both the affinity between an epitope and its antigen binding site on the antigen-binding molecule and the number of pertinent binding sites present on the antigen-binding molecule.

Typically, antigen-binding proteins (such as the polypeptides capable of specifically binding to LRP5 and LRP6) will bind with a dissociation constant (K_{D}) of 10E-5 to 10E-14 moles/liter (M) or less, and preferably 10E-7 to 10E-14 moles/liter (M) or less, more preferably 10E-8 to 10E-14 moles/liter, and even more preferably 10E-11 to 10E-13 (as measured e.g. in a Kinexa assay; known in the art), and/or with an association constant (K_{A}) of at least 10E7 ME-1 preferably at least 10E8 ME-1, more preferably at least 10E9 ME-1, such as at least 10E11 ME-1. Any K_{D} value greater than 10E-4 M is generally considered to indicate non-specific binding. Preferably, a antigen binding protein (such as the polypeptides capable of specifically binding to LRP5 and LRP6) will bind to the desired antigen with a K_{D} less than 500 nM, preferably less than 200 nM, more preferably less than 10 nM, such as less than 500 pM. Specific binding of an antigen-binding protein to an antigen or epitope can be determined in any suitable manner known per se, including, for example, the assays described herein, Scatchard analysis and/or competitive binding assays, such as radioimmunoassays (RIA), enzyme immunoassays (EIA) and sandwich competition assays, and the different variants thereof known per se in the art.

The term “cross-reactive” in connection with binding molecules which are able to bind to LRP5 as well as to LRP6 (“LRP5/LRP6 cross-reactive”) is intended to mean that such binding molecules can specifically bind to an epitope comprised in the LRP5 molecule, and can, alternatively, also specifically bind to an epitope comprised in the LRP6 molecule. Usually, such cross-reactivity may arise in case that the epitopes of the different proteins bound by such binding molecule have a similar structure and/or sequence, e.g. represent conserved epitopes, e.g. are shared by proteins belonging to the same protein family (e.g. LRP5 and LRP6, belonging to the LRP protein family).

The polypeptides capable of specifically binding to LRP5 and LRP6 (also referred to herein as LRP5/LRP6 antagonist(s)) described herein have specificity for LRP5 as well as LRP6, in that they comprise immunoglobulin single variable domains specifically binding to epitopes included in both of these molecules (LRP5/LRP6 cross-reactive binding molecules). They do not, or essentially do not, cross-react with an epitope with a structure similar to the epitopes of LRP5 and LRP6, or with an unrelated structure

When used herein the term “comprising” and variations thereof such as “comprises” and “comprise” can be substituted with the term “containing” or “including” or “having.” Furthermore, the term “comprising” also explicitly encompasses embodiments “consisting of” the recited elements.

Combination Therapy

It is a purpose of the present invention to provide novel therapies for treating or controlling various hyperproliferative diseases, in particular various malignancies.

The inventors of the present application, surprisingly, discovered that the use of an LRP5/LRP6 antagonist in combination with an anti-PD-1 (Programmed cell Death 1) antibody, has the potential to improve clinical outcome compared to the use of a LRP5/LRP6 antagonist or an anti-PD-1 antibody alone.

Specifically, in preclinical studies the inventors tested the immune modulatory function and anti-tumor activity of an LRP5/LRP6 antagonist either alone or in combination with an anti-PD-1 antibody (see Example 1 below). Complete responses, as determined by histopathological analysis, and abundant T cell tumor infiltration was only observed for the combination of the LRP5/LRP6 antagonist with the anti-PD-1 antibody. FACS analysis of the tumor draining lymph nodes further showed that this combination treatment led to an increased number of activated dendritic cells (DCs) in the draining lymph nodes. As further shown in Example 3 below, treatment with Wnt3a ligand of the co-culture of tumor spheroids and activated human PBMCs led to a significant blockade of PBMC-mediated inhibition of tumor cell viability. The treatment with the LRP5/LRP6 antagonist of the co-culture of tumor spheroids and activated human PBMCs in the presence of Wnt3a, restored PBMC-mediated inhibition of tumor cell viability. Combination treatment of the LRP5/LRP6 antagonist and the anti-human PD1 antibody, in accordance with the present invention, leads to the enhancement of PBMC-mediated tumor cell killing, when compared to LRP5/LRP6 antagonist monotherapy.

Without wishing to be bound by theory, these findings indicate that the combination treatment of a LRP5/LRP6 antagonist with an anti-PD-1 antibody leads to inhibition of the Wnt signalling pathway in DCs, which subsequently leads to an upregulation of pro-inflammatory cytokines, restoration of cross-priming and promotion of tumor T cell infiltration and anti-tumour activity.

Although various combination therapies are known in the art and are currently under investigation (e.g. in preclinical or clinical trials), satisfying therapeutic concepts for the treatment of cancer diseases, in particular solid tumors such as lung cancer (e.g. NSCLC), melanoma, bladder and gastrointestinal cancers, are still lacking. Any therapy which shows advantages over standard therapies, such as for example better treatment outcome, beneficial effects, superior efficacy and/or improved tolerability, such as e.g. reduced side effect, would therefore represent an important development.

The surprising results shown in the examples below indicate that the combination of an LRP5/LRP6 antagonist, which on its own had no therapeutic effect in the tumor model, with an anti-PD-1 antibody, which had only a limited therapeutic effect, resulted in a synergistic (i.e. more than additive) interaction of these two compounds that provides for superior results in that a complete response was obtainable.

Thus, the invention relates to methods for the treatment and/or prevention of hyperproliferative diseases, in particular cancer, comprising the combined administration of an LRP5/LRP6 antagonist and an anti-PD-1 antibody, each as described herein, as well as to medical uses, to uses, to pharmaceutical compositions or combinations and kits comprising such therapeutic agents.

Further, the invention relates to anti-cancer therapies comprising using an LRP5/LRP6 antagonist and an anti-PD-1 antibody, each as described herein, in combination.

Such a combined treatment may be given as a non-fixed (e.g. free) combination of the substances or in the form of a fixed combination, including kit-of-parts.

For the treatment of diseases of oncological nature, a large number of anti-cancer agents (including target-specific and non-target-specific anticancer agents) have already been suggested, which can be used as monotherapy or as combination therapy involving more than one agent (e.g. dual or triple combination therapy) and/or which may be combined with radiotherapy (e.g. irradiation treatment), radio-immunotherapy and/or surgery. Therefore, the combined treatment described herein may be given in addition to further therapeutic agents and/or treatments such as radiotherapy, radio-immunotherapy and surgery.

LRP5/LRP6 Antagonist

A polypeptide capable of specifically binding to LRP5 and LRP6 (also referred to herein as an LRP5/LRP6 antagonist) within the meaning of this invention and all of its embodiments is an LRP5/LRP6 cross-reactive biparatopic polypeptide, comprising two or more immunoglobulin single variable domains binding to LRP5 and/or LRP6 at different epitopes. The terms “cross-reactive” and “biparatopic” are explained above, so that LRP5/LRP6 cross-reactive biparatopic molecules can be defined as molecules being able to bind to LRP5 at two different epitopes comprised in the LRP5 protein, and also being able to bind to LRP6 at the corresponding two epitopes comprised in the LRP6 protein.

More specifically, said polypeptide capable of specifically binding to LRP5 and LRP6 include:
a first immunoglobulin single variable domain which is able to specifically bind to LRP5 as well as to LRP6 (LRP5/LRP6 cross-reactive) via an epitope/in a manner that results in inhibition of the Wnt1 signaling pathway, so that Wnt1-driven target gene transcription is inhibited, and
a second immunoglobulin single variable domain which is able to specifically bind to LRP5 as well as to LRP6 (LRP5/LRP6 cross-reactive) via an epitope/in a manner that results in inhibition of the Wnt3a signaling pathway, so that Wnt3a-driven target gene transcription is inhibited.

Due to the two immunoglobulin single variable domains present in such a polypeptide, wherein the two domains bind to different epitopes (Wnt1/Wnt3a signaling related), these molecules are biparatopic binding molecules. In this context, it should be noted that it is assumed that the LRP5/LRP6 antagonists described herein can bind to one single LRP5 or LRP6 molecule via both of its LRP5/LRP6 binding domains. However, other binding modes may occur as well.

In some embodiments of the invention, the polypeptide capable of specifically binding to LRP5 and LRP6 comprises
a first ISVD (a) comprising the following CDR sequences:
   CDR1: TYTVG (=SEQ ID NO:40)
   CDR2: AIRRRGSSTYYADSVKG (=SEQ ID NO:41)
   CDR3: DTRTVALLQYRYDY (=SEQ ID NO:42), and
a second ISVD (b) comprising the following CDR sequences:
   CDR1: SYAMG (=SEQ ID NO:49)
   CDR2: AISWSGGSTYYADSVKG (=SEQ ID NO:50)
   CDR3: SPIPYGSLLRRRNNYDY (=SEQ ID NO:51).

This specific combination of CDR sequences is, for example, contained in the LRP5/LRP6 antagonist termed LRP5/LRP6#1 herein below.

In some embodiments of the invention, the polypeptide capable of specifically binding to LRP5 and LRP6 comprises
a first ISVD (a) comprising the following CDR sequences:
   CDR1: SYAMG (=SEQ ID NO:43)
   CDR2: AIRRSGRRTYYADSVKG (=SEQ ID NO:44)
   CDR3: ARRVRSSTRYNTGTWWWEY (=SEQ ID NO:45), and
a second ISVD (b) comprising the following CDR sequences:
   CDR1: SYAMG (=SEQ ID NO:49)
   CDR2: AISWSGGSTYYADSVKG (=SEQ ID NO:50)
   CDR3: SPIPYGSLLRRRNNYDY (=SEQ ID NO:51).

This specific combination of CDR sequences is, for example, contained in the LRP5/LRP6 antagonist termed LRP5/LRP6#2 herein below.

In some embodiments of the invention, the polypeptide capable of specifically binding to LRP5 and LRP6 comprises
a first ISVD (a) comprising the following CDR sequences:
   CDR1: RYTMG (=SEQ ID NO:46)
   CDR2: AIVRSGGSTYYADSVKG (=SEQ ID NO:47)
   CDR3: DRRGRGENYILLYSSGRYEY (=SEQ ID NO:48), and
a second ISVD (b) comprising the following CDR sequences:
   CDR1: SYAMG (=SEQ ID NO:49)
   CDR2: AISWSGGSTYYADSVKG (=SEQ ID NO:50)
   CDR3: SPIPYGSLLRRRNNYDY (=SEQ ID NO:51).

This specific combination of CDR sequences is, for example, contained in the LRP5/LRP6 antagonist termed LRP5/LRP6#3 herein below.

In some embodiments of the invention, the polypeptide capable of specifically binding to LRP5 and LRP6 comprises
a first ISVD (a) comprising the following CDR sequences:
   CDR1: TYTVG (=SEQ ID NO:40)
   CDR2: AIRRRGSSTYYADSVKG (=SEQ ID NO:41)
   CDR3: DTRTVALLQYRYDY (=SEQ ID NO:42), and
a second ISVD (b) comprising the following CDR sequences:
   CDR1: SYAMG (=SEQ ID NO:52)
   CDR2: AISWRSGSTYYADSVKG (=SEQ ID NO:53)
   CDR3: DPRGYGVAYVSAYYEY (=SEQ ID NO:54).

This specific combination of CDR sequences is, for example, contained in the LRP5/LRP6 antagonist termed LRP5/LRP6#4 herein below.

In some embodiments of the invention, the polypeptide capable of specifically binding to LRP5 and LRP6 comprises
a first ISVD (a) comprising the following CDR sequences:
   CDR1: SYAMG (=SEQ ID NO:43)
   CDR2: AIRRSGRRTYYADSVKG (=SEQ ID NO:44)
   CDR3: ARRVRSSTRYNTGTWWWEY (=SEQ ID NO:45), and
a second ISVD (b) comprising the following CDR sequences:
   CDR1: SYAMG (=SEQ ID NO:52)
   CDR2: AISWRSGSTYYADSVKG (=SEQ ID NO:53)
   CDR3: DPRGYGVAYVSAYYEY (=SEQ ID NO:54).

This specific combination of CDR sequences is, for example, contained in the LRP5/LRP6 antagonist termed LRP5/LRP6#5 herein below.

In some embodiments of the invention, the polypeptide capable of specifically binding to LRP5 and LRP6 comprises
a first ISVD (a) comprising the following CDR sequences:
   CDR1: RYTMG (=SEQ ID NO:46)
   CDR2: AIVRSGGSTYYADSVKG (=SEQ ID NO:47)
   CDR3: DRRGRGENYILLYSSGRYEY (=SEQ ID NO:48), and
a second ISVD (b) comprising the following CDR sequences:
   CDR1: SYAMG (=SEQ ID NO:52)
   CDR2: AISWRSGSTYYADSVKG (=SEQ ID NO:53)
   CDR3: DPRGYGVAYVSAYYEY (=SEQ ID NO:54).

This specific combination of CDR sequences is, for example, contained in the LRP5/LRP6 antagonist termed LRP5/LRP6#6 herein below.

The terms “first” and “second” with respect to such ISVDs or domains in general, as used herein, is solely intended to indicate that these domains are two different domains (as they at least include different CDR sequences). Thus, these terms shall not be understood to refer to the exact order or sequence of the domains within such polypeptide chain. In other words, the above ISVDs (a) and (b) may either be arranged in the order (a)-(b) or in the order (b)-(a) within the polypeptides described herein.

The terms “capable of specifically binding to LRP5 and LRP6” and “specifically bind to LRP5 or LRP6” is intended to mean that the immunoglobulin single variable domains (a) and (b) are cross-reactive with respect to LRP5 and LRP6. Of course, the binding properties of such molecules are determined by their (CDR) sequences, so that the features “capable of specifically binding to LRP5 and LRP6” and “specifically binding to LRP5 or LRP6” set out above and in the claims is only intended to illustrate the utility of the invention, and not to limit the scope of this invention.

Specifically, the ISVDs of the polypeptides described herein (e.g. ISVDs comprising the CDR sequences as defined above) are VHH domains, preferably humanized VHH domains.

In some embodiments of the invention, the polypeptide capable of specifically binding to LRP5 and LRP6 comprises a polypeptide with a first ISVD (a) and a second ISVD (b), said first ISVD comprising a VHH domain with a sequence selected from the group consisting of SEQ ID NO:58, SEQ ID NO:59 and SEQ ID NO:60, and said second ISVD comprising a VHH domain with a sequence selected from the group consisting of SEQ ID NO:61 and SEQ ID NO:62; wherein the sequences are as follows:

SEQ ID NO: 58:
AVQLVESGGGLVQPGGSLRLSCAASGRTFSTYTVGWFRQAPGKEREFVA
AIRRRGSSTYYADSVKGRFTISRDNSKNTVYLQMNSLRPEDTAVYYCAA
DTRTVALLQYRYDYWGQGTLVTVSS
[= Wnt1-333E06mod domain]
SEQ ID NO: 59:
AVQLVESGGGLVQPGGSLRLSCAASGGTFSSYAMGWFRQAPGKEREFVA
AIRRSGRRTYYADSVKGRFTISRDNSKNTVYLQMNSLRPEDTAVYYCAA
ARRVRSSTRYNTGTWWWEYWGQGTLVTVSS
[= Wnt1-333G06 domain]
SEQ ID NO: 60:
AVQLVESGGGLVQPGGSLRLSCAASGLTFSRYTMGWFRQAPGKEREFVA
AIVRSGGSTYYADSVKGRFTISRDNSKNTVYLQMNSLRPEDTAVYYCAA
DRRGRGENYILLYSSGRYEYWGQGTLVTVSS
[= Wnt1-332D03mod domain]
SEQ ID NO: 61:
EVQLVESGGGLVQPGGSLRLSCAASGRTFSSYAMGWFRQAPGKEREFVA
AISWSGGSTYYADSVKGRFTISRDNSKNTVYLQMNSLRPEDTAVYYCAA
SPIPYGSLLRRRNNYDYWGQGTLVTVSS
[= Wnt3a-093A01 domain],
and
SEQ ID NO: 62:
EVQLVESGGGLVQPGGSLRLSCAASGGTFSSYAMGWFRQAPGKEREFVA
AISWRSGSTYYADSVKGRFTISRDNSKNTVYLQMNSLRPEDTAVYYCAA
DPRGYGVAYVSAYYEYWGQGTLVTVSS
[= Wnt3a-367B10 domain]

In some embodiments, the first ISVD comprises the sequence of SEQ ID NO:58 and the second ISVD comprises the sequence of SEQ ID NO:61 (LRP5/LRP6#1).

In some embodiments of the invention, the first ISVD comprises the sequence of SEQ ID NO:59 and the second ISVD comprises the sequence of SEQ ID NO:61 (LRP5/LRP6#2).

In some embodiments, the first ISVD comprises the sequence of SEQ ID NO:60 and the second ISVD comprises the sequence of SEQ ID NO:61 (LRP5/LRP6#3).

In some embodiments, the first ISVD comprises the sequence of SEQ ID NO:58 and the second ISVD comprises the sequence of SEQ ID NO:62 (LRP5/LRP6#4).

In some embodiments, the first ISVD comprises the sequence of SEQ ID NO:59 and the second ISVD comprises the sequence of SEQ ID NO:62 (LRP5/LRP6#5).

In some embodiments, the first ISVD comprises the sequence of SEQ ID NO:60, and the second ISVD comprises the sequence of SEQ ID NO:62 (LRP5/LRP6#6).

In preferred embodiments of the invention, the LRP5/LRP6 antagonist is any of one LRP5/LRP6#1, LRP5/LRP6#5 or LRP5/LRP6#6 as defined by the CDR and/or VHH sequences above.

According to a preferred aspect of the invention, the LRP5/LRP6 antagonist comprises a polypeptide with a first (a) LRP5/LRP6 binding ISVD and a second (b) LRP5/LRP6 binding ISVD and a third ISVD (c). Preferably, the LRP5/LRP6 antagonist comprises a first and second ISVD as defined by the CDR sequences above and a third ISVD, which directly or indirectly links the first and second ISVD. In some embodiments, the first ISVD is covalently linked via a peptide linker to the third ISVD which is covalently linked to the second ISVD via a peptide linker. The two linkers can be identical or different linkers. Also encompassed is that only one linker is present. The terms “first” and “second”, as noted above, do not indicate their position within the polypeptide, thus from N to C terminus the ISVD sequences within the polypeptide can be arranged either in the order ISVDs (a)-(c)-(b), (a)-[linker]-(c)-[linker]-(b), (b)-(c)-(a). (b)-[linker]-(c)-[linker]-(a), (a)-[linker]-(c)-(b), (a)-(c)-[linker]-(b), (b)-[linker]-(c)-(a), (b)-(c)-[linker]-(a).

Preferably, the third ISVD (c) is an albumin binding ISVD. A non-limiting example of such an albumin binding ISVD is the Alb11 domain, comprising the following CDRs:

CDR(Alb11)1: SFGMS (=SEQ ID NO:55)

CDR(Alb11)2: SISGSGSDTLYADSVKG (=SEQ ID NO:56)

CDR(Alb11)3: GGSLSR (=SEQ ID NO:57).

This results in a group of preferred LRP5/LRP6 antagonists having the following structure:

FR(a)1-CDR(a)1-FR(a)2-CDR(a)2-FR(a)3-CDR(a)3-FR(a)4-[optional linker peptide]-FR(Alb11)1-CDR(Alb11)1-FR(Alb11)2-CDR(Alb11)2-FR(Alb11)3-CDR(Alb11)3-FR(Alb11)4-[optional linker peptide]-FR(b)1-CDR(b)1-FR(b)2-CDR(b)2-FR(b)3-CDR(b)3-FR(b)4, preferably wherein the CDRs comprise the sequences as set out above.

Again, the order of the three ISVDs (a), (b), and Alb11 is not fixed but polypeptides in which the above domains are arranged in the order:

(b)-Alb11-(a)

shall be encompassed as well. Furthermore, polypeptides having the Alb11 domain at the N- or C-terminal end of the polypeptide (e.g. Alb11-(a)-(b), Alb11-(b)-(a), (a)-(b)-Alb11, or (b)-(a)-Alb11) shall also be encompassed by the invention.

In some embodiments of the invention, the polypeptide capable of specifically binding to LRP5 and LRP6 comprises
a first ISVD comprising the following CDR sequences:
   CDR1: TYTVG (=SEQ ID NO:40)
   CDR2: AIRRRGSSTYYADSVKG (=SEQ ID NO:41)
   CDR3: DTRTVALLQYRYDY (=SEQ ID NO:42),
a second ISVD comprising the following CDR sequences:
   CDR1: SYAMG (=SEQ ID NO:49)
   CDR2: AISWSGGSTYYADSVKG (=SEQ ID NO:50)
   CDR3: SPIPYGSLLRRRNNYDY (=SEQ ID NO:51), and
an albumin binding ISVD (a third ISVD) comprising the following CDR sequences:
   CDR1: SFGMS (=SEQ ID NO:55)
   CDR2: SISGSGSDTLYADSVKG (=SEQ ID NO:56)
   CDR3: GGSLSR (=SEQ ID NO:57).

This specific combination of CDR sequences is, for example, contained in the LRP5/LRP6 antagonist termed LRP5/LRP6#1 herein below.

In some embodiments of the invention, the polypeptide capable of specifically binding to LRP5 and LRP6 comprises
a first ISVD comprising the following CDR sequences:
   CDR1: SYAMG (=SEQ ID NO:43)
   CDR2: AIRRSGRRTYYADSVKG (=SEQ ID NO:44)
   CDR3: ARRVRSSTRYNTGTWWWEY (=SEQ ID NO:45),
a second ISVD comprising the following CDR sequences:
   CDR1: SYAMG (=SEQ ID NO:49)
   CDR2: AISWSGGSTYYADSVKG (=SEQ ID NO:50)
   CDR3: SPIPYGSLLRRRNNYDY (=SEQ ID NO:51), and
an albumin binding ISVD comprising the following CDR sequences:
   CDR1: SFGMS (=SEQ ID NO:55)
   CDR2: SISGSGSDTLYADSVKG (=SEQ ID NO:56)
   CDR3: GGSLSR (=SEQ ID NO:57).

This specific combination of CDR sequences is, for example, contained in the LRP5/LRP6 antagonist termed LRP5/LRP6#2 herein below.

In some embodiments of the invention, the polypeptide capable of specifically binding to LRP5 and LRP6 comprises
a first ISVD comprising the following CDR sequences:
   CDR1: RYTMG (=SEQ ID NO:46)
   CDR2: AIVRSGGSTYYADSVKG (=SEQ ID NO:47)
   CDR3: DRRGRGENYILLYSSGRYEY (=SEQ ID NO:48),
a second ISVD with the following CDR sequences:
   CDR1: SYAMG (=SEQ ID NO:49)
   CDR2: AISWSGGSTYYADSVKG (=SEQ ID NO:50)
   CDR3: SPIPYGSLLRRRNNYDY (=SEQ ID NO:51), and
an albumin binding ISVD comprising the following CDR sequences:
   CDR1: SFGMS (=SEQ ID NO:55)
   CDR2: SISGSGSDTLYADSVKG (=SEQ ID NO:56)
   CDR3: GGSLSR (=SEQ ID NO:57).

This specific combination of CDR sequences is, for example, contained in the LRP5/LRP6 antagonist termed LRP5/LRP6#3 herein below.

In some embodiments of the invention, the polypeptide capable of specifically binding to LRP5 and LRP6 comprises
a first ISVD comprising the following CDR sequences:
   CDR1: TYTVG (=SEQ ID NO:40)
   CDR2: AIRRRGSSTYYADSVKG (=SEQ ID NO:41)
   CDR3: DTRTVALLQYRYDY (=SEQ ID NO:42),
a second ISVD comprising the following CDR sequences:
   CDR1: SYAMG (=SEQ ID NO:52)
   CDR2: AISWRSGSTYYADSVKG (=SEQ ID NO:53)
   CDR3: DPRGYGVAYVSAYYEY (=SEQ ID NO:54), and
an albumin binding ISVD comprising the following CDR sequences:
   CDR1: SFGMS (=SEQ ID NO:55)
   CDR2: SISGSGSDTLYADSVKG (=SEQ ID NO:56)
   CDR3: GGSLSR (=SEQ ID NO:57).

This specific combination of CDR sequences is, for example, contained in the LRP5/LRP6 antagonist termed LRP5/LRP6#4 herein below.

In some embodiments of the invention, the polypeptide capable of specifically binding to LRP5 and LRP6 comprises
a first ISVD comprising the following CDR sequences:
   CDR1: SYAMG (=SEQ ID NO:43)
   CDR2: AIRRSGRRTYYADSVKG (=SEQ ID NO:44)
   CDR3: ARRVRSSTRYNTGTWWWEY (=SEQ ID NO:45),
a second ISVD comprising the following CDR sequences:
   CDR1: SYAMG (=SEQ ID NO:52)
   CDR2: AISWRSGSTYYADSVKG (=SEQ ID NO:53)
   CDR3: DPRGYGVAYVSAYYEY (=SEQ ID NO:54), and
an albumin binding ISVD comprising the following CDR sequences:
   CDR1: SFGMS (=SEQ ID NO:55)
   CDR2: SISGSGSDTLYADSVKG (=SEQ ID NO:56)
   CDR3: GGSLSR (=SEQ ID NO:57).

This specific combination of CDR sequences is, for example, contained in the LRP5/LRP6 antagonist termed LRP5/LRP6#5 herein below.

In some embodiments of the invention, the polypeptide capable of specifically binding to LRP5 and LRP6 comprises
a first ISVD comprising the following CDR sequences:
   CDR1: RYTMG (=SEQ ID NO:46)
   CDR2: AIVRSGGSTYYADSVKG (=SEQ ID NO:47)
   CDR3: DRRGRGENYILLYSSGRYEY (=SEQ ID NO:48),
a second ISVD comprising the following CDR sequences:
   CDR1: SYAMG (=SEQ ID NO:52)
   CDR2: AISWRSGSTYYADSVKG (=SEQ ID NO:53)
   CDR3: DPRGYGVAYVSAYYEY (=SEQ ID NO:54), and
an albumin binding ISVD comprising the following CDR sequences:
   CDR1: SFGMS (=SEQ ID NO:55)
   CDR2: SISGSGSDTLYADSVKG (=SEQ ID NO:56)
   CDR3: GGSLSR (=SEQ ID NO:57).

This specific combination of CDR sequences is, for example, contained in the LRP5/LRP6 antagonist termed LRP5/LRP6#6 herein below.

In some embodiments, the ISVDs as defined by their CDR sequences in the above polypeptides capable of specifically binding to LRP5 and LRP6 are arranged such that the albumin binding ISVD directly or indirectly (e.g. via (a) linker peptide(s)) links the first and the second ISVD.

The sequence of the above-mentioned Alb11 immunoglobulin single variable domain is as follows:

EVQLVESGGGLVQPGNSLRLSCAASGFTFSSFGMSWVRQAPGKGLEWVS
SISGSGSDTLYADSVKGRFTISRDNAKTTLYLQMNSLRPEDTAVYYCTI
GGSLSRSSQGTLVTVSS
(= Alb11 domain; = SEQ ID NO: 63)

The CDR sequences mentioned above are summarized in Tables 1A, 1B, and 1C:

TABLE 1A
CDR sequences of immunoglobulin
single variable domains interfering
with Wnt1 signaling:

| | Wnt1- | Wnt1- | Wnt1- |
|---|---|---|---|
| | 333E06mod | 333G06 | 332D03mod |
| CDR1 | TYTVG | SYAMG | RYTMG |
| | (SEQ ID | (SEQ ID | (SEQ ID |
| | NO: 40) | NO: 43) | NO: 46) |
| CDR2 | AIRRRGSST | AIRRSGRRT | AIVRSGGST |
| | YYADSVKG | YYADSVKG | YYADSVKG |
| | (SEQ ID | (SEQ ID | (SEQ ID |
| | NO: 41) | NO: 44) | NO: 47) |
| CDR3 | DTRTVALLQ | ARRVRSSTRY | DRRGRGENYI |
| | YRYDY | NTGTWWWEY | LLYSSGRYEY |
| | (SEQ ID | (SEQ ID | (SEQ ID |
| | NO: 42) | NO: 45) | NO: 48) |

TABLE 1B
CDR sequences of immunoglobulin single variable
domains interfering with Wnt3a signaling:

| | Wnt3a-093A01 | Wnt3a-367B10 |
|---|---|---|
| CDR1 | SYAMG | SYAMG |
| | (SEQ ID NO: 49) | (SEQ ID NO: 52) |
| CDR2 | AISWSGGSTYYADSVKG | AISWRSGSTYYADSVKG |
| | (SEQ ID NO: 50) | (SEQ ID NO: 53) |
| CDR3 | SPIPYGSLLRRRNNYDY | DPRGYGVAYVSAYYEY |
| | (SEQ ID NO: 51) | (SEQ ID NO: 54) |

TABLE 1C
CDR sequences of immunoglobulin single variable
domain binding to serum albumin (Alb11 domain):

| | Alb11 domain |
|---|---|
| CDR1 | SFGMS |
| | (SEQ ID NO: 55) |
| CDR2 | SISGSGSDTLYADSVKG |
| | (SEQ ID NO: 56) |
| CDR3 | GGSLSR |
| | (SEQ ID NO: 57) |

Three preferred LRP5/LRP6 antagonist described herein are as follows: First preferred LRP5/LRP6 antagonist: Polypeptides comprising
a first (LRP5/LRP6 binding) ISVD comprising the amino acid sequence as shown in SEQ ID NO:58;
an albumin binding ISVD comprising the amino acid sequence as shown in SEQ ID NO:63;
a second (LRP5/LRP6 binding) ISVD comprising the amino acid sequence as shown in SEQ ID NO:61;

either in this order, or the order of the above three domains being changed.

Second preferred LRP5/LRP6 antagonist: Polypeptides comprising
a first (LRP5/LRP6 binding) ISVD comprising the amino acid sequence as shown in SEQ ID NO:59;
an albumin binding ISVD comprising the amino acid sequence as shown in SEQ ID NO:63;
a second (LRP5/LRP6 binding) ISVD comprising the amino acid sequence as shown in SEQ ID NO:62;

either in this order, or the order of the above three domains being changed.

Third preferred LRP5/LRP6 antagonist: Polypeptides comprising
a first (LRP5/LRP6 binding) ISVD comprising the amino acid sequence as shown in SEQ ID NO:60;
an albumin binding ISVD comprising the amino acid sequence as shown in SEQ ID NO:63;
a second (LRP5/LRP6 binding) ISVD comprising the amino acid sequence as shown in SEQ ID NO:62;

either in this order, or the order of the above three domains being changed.

In even more specifically preferred embodiments, the albumin binding ISVD is located between the two LRP5/LRP6 binding ISVDs.

The sequences of the VHHs mentioned above are summarized in Tables 2A, 2B, and 2C:

TABLE 2A
Sequences of immunoglobulin single variable
domains interfering with Wnt1 signaling:

| SEQ ID NO: | VHH sequences |
|---|---|
| Wnt1- | AVQLVESGGGLVQPGGSLRLSCAASGRTFSTYT |
| 333E06mod | VGWFRQAPGKEREFVAAIRRRGSSTYYADSVKG |
| SEQ ID NO: 58 | RFTISRDNSKNTVYLQMNSLRPEDTAVYYCAAD |
| | TRTVALLQYRYDYWGQGTLVTVSS |
| Wnt1- | AVQLVESGGGLVQPGGSLRLSCAASGGTFSSYA |
| 333G06 | MGWFRQAPGKEREFVAAIRRSGRRTYYADSVKG |
| SEQ ID NO: 59 | RFTISRDNSKNTVYLQMNSLRPEDTAVYYCAAA |
| | RRVRSSTRYNTGTWWWEYWGQGTLVTVSS |
| Wnt1- | AVQLVESGGGLVQPGGSLRLSCAASGLTFSRYT |
| 332D03mod | MGWFRQAPGKEREFVAAIVRSGGSTYYADSVKG |
| SEQ ID NO: 60 | RFTISRDNSKNTVYLQMNSLRPEDTAVYYCAAD |
| | RRGRGENYILLYSSGRYEYWGQGTLVTVSS |

TABLE 2B
Sequences of immunoglobulin single variable
domains interfering with Wnt3a signaling:

| SEQ ID NO: | VHH sequences |
|---|---|
| Wnt3a- | EVQLVESGGGLVQPGGSLRLSCAASGRTFSSYA |
| 093A01 | MGWFRQAPGKEREFVAAISWSGGSTYYADSVKG |
| SEQ ID NO: 61 | RFTISRDNSKNTVYLQMNSLRPEDTAVYYCAAS |
| | PIPYGSLLRRRNNYDYWGQGTLVTVSS |
| Wnt3a- | EVQLVESGGGLVQPGGSLRLSCAASGGTFSSYA |
| 367B10 | MGWFRQAPGKEREFVAAISWRSGSTYYADSVKG |
| SEQ ID NO: 62 | RFTISRDNSKNTVYLQMNSLRPEDTAVYYCAAD |
| | PRGYGVAYVSAYYEYWGQGTLVTVSS |

TABLE 2C
Sequence of immunoglobulin single variable
domain binding to serum albumin (Alb11 domain):

| SEQ ID NO: | VHH sequences |
|---|---|
| Alb11 | EVQLVESGGGLVQPGNSLRLSCAASGFTFSSFG |
| SEQ ID NO: 63 | MSWVRQAPGKGLEWVSSISGSGSDTLYADSVKG |
| | RFTISRDNAKTTLYLQMNSLRPEDTAVYYCTIG |
| | GSLSRSSQGTLVTVSS |

In preferred embodiments of the invention, the LRP5/LRP6 antagonist comprises a sequence selected from SEQ ID NOs: 64, 65 and 66 (these preferred polypeptides capable of specifically binding to LRP5 and LRP6 are also referred to herein as LRP5/LRP6#1, LRP5/LRP6#5 and LRP5/LRP6#6, respectively), wherein the exact amino acid sequences can be taken from Table 2D below:

TABLE 2D
Sequences of three specific embodiments
of polypeptides capable of specifically
binding to LRP5 and LRP6

| | Amino Acid Sequence |
|---|---|
| SEQ ID NO: | (CDR sequences underlined) |
| SEQ ID NO: 64 | AVQLVESGGGLVQPGGSLRLSCAASGRIFSTYTVG |
| | WFRQAPGKEREFVAAIRRRGSSTYYADSVKGRFTI |
| | SRDNSKNTVYLQMNSLRPEDTAVYYCAADTRTVAL |
| | LQYRYDYWGQGTLVTVSSGGGGSGGGGSGGGGSGG |
| | GGSGGGGSGGGGSGGGGSEVQLVESGGGLVQPGNS |
| | LRLSCAASGFTFSSFGMSWVRQAPGKGLEWVSSIS |
| | GSGSDTLYADSVKGRFTISRDNAKTTLYLQMNSLR |
| | PEDTAVYYCTIGGSLSRSSQGTLVTVSSGGGGSGG |
| | GGSGGGGSGGGGSGGGGSGGGGSGGGGSEVQLVES |
| | GGGLVQPGGSLRLSCAASGRTFSSYAMGWFRQAPG |
| | KEREFVAAISWSGGSTYYADSVKGRFTISRDNSKN |
| | TVYLQMNSLRPEDTAVYYCAASPIPYGSLLRRRNN |
| | YDYWGQGTLVTVSSA |
| SEQ ID NO: 65 | AVQLVESGGGLVQPGGSLRLSCAASGGIFSSYAMG |
| | WFRQAPGKEREFVAAIRRSGRRTYYADSVKGRFTI |
| | SRDNSKNTVYLQMNSLRPEDTAVYYCAAARRVRSS |
| | TRYNTGTWWWEYWGQGTLVTVSSGGGGSGGGGSGG |
| | GGSGGGGSGGGGSGGGGSGGGGSEVQLVESGGGLV |
| | QPGNSLRLSCAASGFTFSSFGMSWVRQAPGKGLEW |
| | VSSISGSGSDTLYADSVKGRFTISRDNAKTTLYLQ |
| | MNSLRPEDTAVYYCTIGGSLSRSSQGTLVTVSSGG |
| | GGSGGGGSGGGGSGGGGSGGGGSGGGGSGGGGSEV |
| | QLVESGGGLVQPGGSLRLSCAASGGTFSSYAMGWF |
| | RQAPGKEREFVAAISWRSGSTYYADSVKGRFTISR |
| | DNSKNTVYLQMNSLRPEDTAVYYCAADPRGYGVAY |
| | VSAYYEYWGQGTLVTVSSA |
| SEQ ID NO: 66 | AVQLVESGGGLVQPGGSLRLSCAASGLIFSRYTMG |
| | WFRQAPGKEREFVAAIVRSGGSTYYADSVKGRFTI |
| | SRDNSKNTVYLQMNSLRPEDTAVYYCAADRRGRGE |
| | NYILLYSSGRYEYWGQGTLVTVSSGGGGSGGGGSG |
| | GGGSGGGGSGGGGSGGGGSGGGGSEVQLVESGGGL |
| | VQPGNSLRLSCAASGFTFSSFGMSWVRQAPGKGLE |
| | WVSSISGSGSDTLYADSVKGRFTISRDNAKTTLYL |
| | QMNSLRPEDTAVYYCTIGGSLSRSSQGTLVTVSSG |
| | GGGSGGGGSGGGGSGGGGSGGGGSGGGGSGGGGSE |
| | VQLVESGGGLVQPGGSLRLSCAASGGTFSSYAMGW |
| | FRQAPGKEREFVAAISWRSGSTYYADSVKGRFTIS |
| | RDNSKNTVYLQMNSLRPEDTAVYYCAADPRGYGVA |
| | YVSAYYEYWGQGTLVTVSSA |

Manufacture and therapeutic use of the aforementioned polypeptides capable of specifically binding to LRP5 and LRP6 is disclosed in WO2017/093478A1. In particular, this document provides a sufficient disclosure of the method of preparing the polypeptides capable of specifically binding to LRP5 and LRP6 used in the present invention.

Anti-PD-1 Antibody

An anti-PD-1 antibody (also referred to as “PD-1 antibody” herein) within the meaning of this invention and all of its embodiments is a compound that inhibits the interaction of PD-1 with its ligand(s), Preferably, the anti-PD-1 antibody is a humanized or fully human anti-PD-1 antibody. Any one of these antibodies may be a recombinant human antibody.

The PD-1 gene encodes a 55 kDa type I transmembrane protein that is part of the Ig gene superfamily (Agata et al. (1996) Int Immunol. 8:765-72). The complete PD-1 sequence can be found under GenBank Accession No. U64863. Although structurally similar to CTLA-4, PD-1 lacks the MYPPY motif (SEQ ID NO:39) that is important for B7-1 and B7-2 binding.

PD-1 is an inhibitory member of the extended CD28/CTLA-4 family of T cell regulators. Other members of the CD28 family include CD28, CTLA-4, ICOS and BTLA. PD-1 is suggested to exist as a monomer, lacking the unpaired cysteine residue characteristic of other CD28 family members. PD-1 is expressed on activated B cells, T cells, and monocytes (Okazaki et al. (2002) Curr Opin Immunol 14:391779-82; Bennett et al. (2003) J. Immunol. 170:711-8). Two ligands for PD-1 have been identified, PD-L1 (B7-H1) and PD-L2 (B7-DC), that have been shown to downregulate T cell activation upon binding to PD-1 (Freeman et al. (2000) J. Exp. Med. 192:1027-34; Carter et al. (2002) Eur. J. Immunol. 32:634-43). Both PD-L1 and PD-L2 are B7 homologs that bind to PD-1. PD-L1 is abundant in a variety of human cancers (Dong et al. (2002) Nat. Med. 8:787-9).

PD-1 is known as an immuno-inhibitory protein that negatively regulates TCR signals (Ishida, Y. et al. (1992) EMBO J. 11:3887-3895; Blank, C. et al. (2006) Immunol. Immunother. 56(6):739-745). The interaction between PD-1 and PD-L1 can act as an immune checkpoint, which can lead to, e.g., a decrease in tumor infiltrating lymphocytes, a decrease in T-cell receptor mediated proliferation, and/or immuno-evasion by cancerous cells (Dong et al. (2003) J. Mol. Med. 81:281-7; Blank et al. (2005) Cancer Immunol. Immunother. 54:307-314; Konishi et al. (2004) Clin. Cancer Res. 10:5094-100). Immune suppression can be reversed by inhibiting the local interaction of PD-1 with PD-L1 or PD-L2; the effect is additive when the interaction of PD-1 with both PD-L1 and PD-L2 is blocked (Iwai et al. (2002) Proc. Nat'l. Acad. Sci USA 99:12293-7; Brown et al. (2003) J. Immunol. 170:1257-66).

In one aspect of the invention, the anti-PD-1 antibody is any one of antibodies PD1-1, PD1-2, PD-1-3, PD1-4 and PD1-5 defined by the sequences as shown in Table 3 by way of the SEQ ID numbers, wherein VH denotes the heavy chain variable domain, VL denotes the light chain variable domain, HC denotes the (full length) heavy chain and LC denotes the (full length) light chain:

TABLE 3
SEQ ID NOs of the CDR, VH, VL, HC and LC sequences

| anti-PD1 | CDR | VH | VL | HC | LC |
|---|---|---|---|---|---|
| antibody | sequences | sequences | sequences | sequences | sequences |
| PD1-1 | 1-6 | 19 | 20 | 29 | 30 |
| PD1-2 | 7-12 | 21 | 22 | 31 | 32 |
| PD1-3 | 13-18 | 23 | 24 | 33 | 34 |
| PD1-4 | 13-18 | 25 | 26 | 35 | 36 |
| PD1-5 | 13-18 | 27 | 28 | 37 | 38 |

and wherein the amino acid sequences (and sequence names) of the SEQ ID numbers are as shown in Table 4:

TABLE 4

| SEQ | Sequence | |
|---|---|---|
| ID NO: | name | Amino acid sequence |
| 1 | PD1-1HCDR1 | GFTFSASAMS |
| 2 | PD1-1HCDR2 | YISGGGGDTYYSSSVKG |
| 3 | PD1-1HCDR3 | HSNVNYYAMDY |
| 4 | PD1-1LCDR1 | RASENIDTSGISFMN |
| 5 | PD1-1LCDR2 | VASNQGS |
| 6 | PD1-1LCDR3 | QQSKEVPWT |
| 7 | PD1-2HCDR1 | GFTFSASAMS |
| 8 | PD1-2HCDR2 | YISGGGGDTYYSSSVKG |
| 9 | PD1-2HCDR3 | HSNPNYYAMDY |
| 10 | PD1-2LCDR1 | RASENIDTSGISFMN |
| 11 | PD1-2LCDR2 | VASNQGS |
| 12 | PD1-2LCDR3 | QQSKEVPWT |
| 13 | PD1-3HCDR1 | GFTFSKSAMS |
| 14 | PD1-3HCDR2 | YISGGGGDTYYSSSVKG |
| 15 | PD1-3HCDR3 | HSNVNYYAMDY |
| 16 | PD1-3LCDR1 | RASENIDVSGISFMN |
| 17 | PD1-3LCDR2 | VASNQGS |
| 18 | PD1-3LCDR3 | QQSKEVPWT |
| 19 | PD1VH1 | EVMLVESGGGLVQPGGSLRLSCTASGFTFS |
| | | ASAMSWVRQAPGKGLEWVAYISGGGGDTYY |
| | | SSSVKGRFTISRDNAKNSLYLQMNSLRAED |
| | | TAVYYCARHSNVNYYAMDYWGQGTLVTVSS |
| 20 | PD1VL1 | EIVLTQSPATLSLSPGERATMSCRASENID |
| | | TSGISFMNWYQQKPGQAPKLLIYVASNQGS |
| | | GIPARFSGSGSGTDFTLTISRLEPEDFAVY |
| | | YCQQSKEVPWTFGQGTKLEIK |
| 21 | PD1VH2 | EVMLVESGGGLVQPGGSLRLSCTASGFTFS |
| | | ASAMSWVRQAPGKGLEWVAYISGGGGDTYY |
| | | SSSVKGRFTISRDNAKNSLYLQMNSLRAED |
| | | TAVYYCARHSNPNYYAMDYWGQGTLVTVSS |
| 22 | PD1VL2 | EIVLTQSPATLSLSPGERATMSCRASENID |
| | | TSGISFMNWYQQKPGQAPKLLIYVASNQGS |
| | | GIPARFSGSGSGTDFTLTISRLEPEDFAVY |
| | | YCQQSKEVPWTFGQGTKLEIK |
| 23 | PD1VH3 | EVMLVESGGGLVQPGGSLRLSCTASGFTFS |
| | | KSAMSWVRQAPGKGLEWVAYISGGGGDTYY |
| | | SSSVKGRFTISRDNAKNSLYLQMNSLRAED |
| | | TAVYYCARHSNVNYYAMDYWGQGTLVTVSS |
| 24 | PD1VL3 | EIVLTQSPATLSLSPGERATMSCRASENID |
| | | VSGISFMNWYQQKPGQAPKLLIYVASNQGS |
| | | GIPARFSGSGSGTDFTLTISRLEPEDFAVY |
| | | YCQQSKEVPWTFGQGTKLEIK |
| 25 | PD1VH4 | EVMLVESGGGLVQPGGSLRLSCTASGFTFS |
| | | KSAMSWVRQAPGKGLEWVAYISGGGGDTYY |
| | | SSSVKGRFTISRDNAKNSLYLQMNSLRAED |
| | | TAVYYCARHSNVNYYAMDYWGQGTLVTVSS |
| 26 | PD1VL4 | EIVLTQSPATLSLSPGERATMSCRASENID |
| | | VSGISFMNWYQQKPGQAPKLLIYVASNQGS |
| | | GIPARFSGSGSGTDFTLTISRLEPEDFAVY |
| | | YCQQSKEVPWTFGQGTKLEIK |
| 27 | PD1VH5 | EVMLVESGGGLVQPGGSLRLSCTASGFTFS |
| | | KSAMSWVRQAPGKGLEWVAYISGGGGDTYY |
| | | SSSVKGRFTISRDNAKNSLYLQMNSLRAED |
| | | TAVYYCARHSNVNYYAMDYWGQGTLVTVSS |
| 28 | PD1VL5 | EIVLTQSPATLSLSPGERATMSCRASENID |
| | | VSGISFMNWYQQKPGQAPKLLIYVASNQGS |
| | | GIPARFSGSGSGTDFTLTISRLEPEDFAVY |
| | | YCQQSKEVPWTFGQGTKLEIK |
| 29 | PD1HC1 | EVMLVESGGGLVQPGGSLRLSCTASGFTFS |
| | | ASAMSWVRQAPGKGLEWVAYISGGGGDTYY |
| | | SSSVKGRFTISRDNAKNSLYLQMNSLRAED |
| | | TAVYYCARHSNVNYYAMDYWGQGTLVTVSS |
| | | ASTKGPSVFPLAPCSRSTSESTAALGCLVK |
| | | DYFPEPVTVSWNSGALTSGVHTFPAVLQSS |
| | | GLYSLSSVVTVPSSSLGTKTYTCNVDHKPS |
| | | NTKVDKRVESKYGPPCPPCPAPEFLGGPSV |
| | | FLFPPKPKDTLMISRTPEVTCVVVDVSQED |
| | | PEVQFNWYVDGVEVHNAKTKPREEQFNSTY |
| | | RVVSVLTVLHQDWLNGKEYKCKVSNKGLPS |
| | | SIEKTISKAKGQPREPQVYTLPPSQEEMTK |
| | | NQVSLTCLVKGFYPSDIAVEWESNGQPENN |
| | | YKTTPPVLDSDGSFFLYSRLTVDKSRWQEG |
| | | NVFSCSVMHEALHNHYTQKSLSLSLG |
| 30 | PD1LC1 | EIVLTQSPATLSLSPGERATMSCRASENID |
| | | TSGISFMNWYQQKPGQAPKLLIYVASNQGS |
| | | GIPARFSGSGSGTDFTLTISRLEPEDFAVY |
| | | YCQQSKEVPWTFGQGTKLEIKRTVAAPSVF |
| | | IFPPSDEQLKSGTASVVCLLNNFYPREAKV |
| | | QWKVDNALQSGNSQESVTEQDSKDSTYSLS |
| | | STLTLSKADYEKHKVYACEVTHQGLSSPVT |
| | | KSFNRGEC |
| 31 | PD1HC2 | EVMLVESGGGLVQPGGSLRLSCTASGFTFS |
| | | ASAMSWVRQAPGKGLEWVAYISGGGGDTYY |
| | | SSSVKGRFTISRDNAKNSLYLQMNSLRAED |
| | | TAVYYCARHSNPNYYAMDYWGQGTLVTVSS |
| | | ASTKGPSVFPLAPCSRSTSESTAALGCLVK |
| | | DYFPEPVTVSWNSGALTSGVHTFPAVLQSS |
| | | GLYSLSSVVTVPSSSLGTKTYTCNVDHKPS |
| | | NTKVDKRVESKYGPPCPPCPAPEFLGGPSV |
| | | FLFPPKPKDTLMISRTPEVTCVVVDVSQED |
| | | PEVQFNWYVDGVEVHNAKTKPREEQFNSTY |
| | | RVVSVLTVLHQDWLNGKEYKCKVSNKGLPS |
| | | SIEKTISKAKGQPREPQVYTLPPSQEEMTK |
| | | NQVSLTCLVKGFYPSDIAVEWESNGQPENN |
| | | YKTTPPVLDSDGSFFLYSRLTVDKSRWQEG |
| | | NVFSCSVMHEALHNHYTQKSLSLSLG |
| 32 | PD1LC2 | EIVLTQSPATLSLSPGERATMSCRASENID |
| | | TSGISFMNWYQQKPGQAPKLLIYVASNQGS |
| | | GIPARFSGSGSGTDFTLTISRLEPEDFAVY |
| | | YCQQSKEVPWTFGQGTKLEIKRTVAAPSVF |
| | | IFPPSDEQLKSGTASVVCLLNNFYPREAKV |
| | | QWKVDNALQSGNSQESVTEQDSKDSTYSLS |
| | | STLTLSKADYEKHKVYACEVTHQGLSSPVT |
| | | KSFNRGEC |
| 33 | PD1HC3 | EVMLVESGGGLVQPGGSLRLSCTASGFTFS |
| | | KSAMSWVRQAPGKGLEWVAYISGGGGDTYY |
| | | SSSVKGRFTISRDNAKNSLYLQMNSLRAED |
| | | TAVYYCARHSNVNYYAMDYWGQGTLVTVSS |
| | | ASTKGPSVFPLAPCSRSTSESTAALGCLVK |
| | | DYFPEPVTVSWNSGALTSGVHTFPAVLQSS |
| | | GLYSLSSVVTVPSSSLGTKTYTCNVDHKPS |
| | | NTKVDKRVESKYGPPCPPCPAPEFLGGPSV |
| | | FLFPPKPKDTLMISRTPEVTCVVVDVSQED |
| | | PEVQFNWYVDGVEVHNAKTKPREEQFNSTY |
| | | RVVSVLTVLHQDWLNGKEYKCKVSNKGLPS |
| | | SIEKTISKAKGQPREPQVYTLPPSQEEMTK |
| | | NQVSLTCLVKGFYPSDIAVEWESNGQPENN |
| | | YKTTPPVLDSDGSFFLYSRLTVDKSRWQEG |
| | | NVFSCSVMHEALHNHYTQKSLSLSLG |
| 34 | PD1LC3 | EIVLTQSPATLSLSPGERATMSCRASENID |
| | | VSGISFMNWYQQKPGQAPKLLIYVASNQGS |
| | | GIPARFSGSGSGTDFTLTISRLEPEDFAVY |
| | | YCQQSKEVPWTFGQGTKLEIKRTVAAPSVF |
| | | IFPPSDEQLKSGTASVVCLLNNFYPREAKV |
| | | QWKVDNALQSGNSQESVTEQDSKDSTYSLS |
| | | STLTLSKADYEKHKVYACEVTHQGLSSPVT |
| | | KSFNRGEC |
| 35 | PD1HC4 | EVMLVESGGGLVQPGGSLRLSCTASGFTFS |
| | | KSAMSWVRQAPGKGLEWVAYISGGGGDTYY |
| | | SSSVKGRFTISRDNAKNSLYLQMNSLRAED |
| | | TAVYYCARHSNVNYYAMDYWGQGTLVTVSS |
| | | ASTKGPSVFPLAPCSRSTSESTAALGCLVK |
| | | DYFPEPVTVSWNSGALTSGVHTFPAVLQSS |
| | | GLYSLSSVVTVPSSSLGTKTYTCNVDHKPS |
| | | NTKVDKRVESKYGPPCPPCPAPEFLGGPSV |
| | | FLFPPKPKDTLMISRTPEVTCVVVDVSQED |
| | | PEVQFNWYVDGVEVHNAKTKPREEQFNSTY |
| | | RVVSVLTVLHQDWLNGKEYKCKVSNKGLPS |
| | | SIEKTISKAKGQPREPQVYTLPPSQEEMTK |
| | | NQVSLTCLVKGFYPSDIAVEWESNGQPENN |
| | | YKTTPPVLDSDGSFFLYSRLTVDKSRWQEG |
| | | NVFSCSVMHEALHNHYTQKSLSLSLG |
| 36 | PD1LC4 | EIVLTQSPATLSLSPGERATMSCRASENID |
| | | VSGISFMNWYQQKPGQAPKLLIYVASNQGS |
| | | GIPARFSGSGSGTDFTLTISRLEPEDFAVY |
| | | YCQQSKEVPWTFGQGTKLEIKRTVAAPSVF |
| | | IFPPSDEQLKSGTASVVCLLNNFYPREAKV |
| | | QWKVDNALQSGNSQESVTEQDSKDSTYSLS |
| | | STLTLSKADYEKHKVYACEVTHQGLSSPVT |
| | | KSFNRGEC |
| 37 | PD1HC5 | EVMLVESGGGLVQPGGSLRLSCTASGFTFS |
| | | KSAMSWVRQAPGKGLEWVAYISGGGGDTYY |
| | | SSSVKGRFTISRDNAKNSLYLQMNSLRAED |
| | | TAVYYCARHSNVNYYAMDYWGQGTLVTVSS |
| | | ASTKGPSVFPLAPCSRSTSESTAALGCLVK |
| | | DYFPEPVTVSWNSGALTSGVHTFPAVLQSS |
| | | GLYSLSSVVTVPSSSLGTKTYTCNVDHKPS |
| | | NTKVDKRVESKYGPPCPPCPAPEFLGGPSV |
| | | FLFPPKPKDTLMISRTPEVTCVVVDVSQED |
| | | PEVQFNWYVDGVEVHNAKTKPREEQFNSTY |
| | | RVVSVLTVLHQDWLNGKEYKCKVSNKGLPS |
| | | SIEKTISKAKGQPREPQVYTLPPSQEEMTK |
| | | NQVSLTCLVKGFYPSDIAVEWESNGQPENN |
| | | YKTTPPVLDSDGSFFLYSRLTVDKSRWQEG |
| | | NVFSCSVMHEALHNHYTQKSLSLSLG |
| 38 | PD1LC5 | EIVLTQSPATLSLSPGERATMSCRASENID |
| | | VSGISFMNWYQQKPGQAPKLLIYVASNQGS |
| | | GIPARFSGSGSGTDFTLTISRLEPEDFAVY |
| | | YCQQSKEVPWTFGQGTKLEIKRTVAAPSVF |
| | | IFPPSDEQLKSGTASVVCLLNNFYPREAKV |
| | | QWKVDNALQSGNSQESVTEQDSKDSTYSLS |
| | | STLTLSKADYEKHKVYACEVTHQGLSSPVT |
| | | KSFNRGEC |

Specifically, an anti-PD-1 antibody molecule described herein comprises: (a) heavy chain CDRs comprising the amino acid sequence of SEQ ID NO:1 (HCDR1), SEQ ID NO:2 (HCDR2) and SEQ ID NO:3 (HCDR3) and light chain CDRs comprising the amino acid sequence of SEQ ID NO:4 (LCDR1), SEQ ID NO:5 (LCDR2) and SEQ ID NO:6 (LCDR3); or, b) heavy chain CDRs comprising the amino acid sequence of SEQ ID NO:7 (HCDR1), SEQ ID NO:8 (HCDR2) and SEQ ID NO:9 (HCDR3) and light chain CDRs comprising the amino acid sequence of SEQ ID NO:10 (LCDR1), SEQ ID NO:11 (LCDR2) and SEQ ID NO:12 (LCDR3); or (c) heavy chain CDRs comprising the amino acid sequence of SEQ ID NO:13 (HCDR1), SEQ ID NO:14 (HCDR2) and SEQ ID NO:15 (HCDR3) and light chain CDRs comprising the amino acid sequence of SEQ ID NO:16 (LCDR1), SEQ ID NO:17 (LCDR2) and SEQ ID NO:18 (LCDR3).

In some embodiments, the anti-PD-1 antibody molecule comprises a heavy chain variable domain comprising an amino acid sequence selected from SEQ ID NOs: 19, 21, 23, 25 and 27.

In some embodiments, the anti-PD-1 antibody molecule comprises a light chain variable domain comprising an amino acid sequence selected from SEQ ID NOs: 20, 22, 24, 26 and 28.

In some embodiments, the anti-PD-1 antibody molecule comprises (a) a heavy chain variable domain comprising the amino acid sequence of SEQ ID NO: 19 and a light chain variable domain comprising the amino acid sequence of SEQ ID NO: 20, (b) a heavy chain variable domain comprising the amino acid sequence of SEQ ID NO: 21 and a light chain variable domain comprising the amino acid sequence of SEQ ID NO: 22, (c) a heavy chain variable domain comprising the amino acid sequence of SEQ ID NO: 23 and a light chain variable domain comprising the amino acid sequence of SEQ ID NO: 24, (d) a heavy chain variable domain comprising the amino acid sequence of SEQ ID NO: 25 and a light chain variable domain comprising the amino acid sequence of SEQ ID NO: 26, or (e) a heavy chain variable domain comprising the amino acid sequence of SEQ ID NO: 27 and a light chain variable domain comprising the amino acid sequence of SEQ ID NO: 28.

In some embodiments, the anti-PD-1 antibody comprises (a) a heavy chain comprising the amino acid sequence of SEQ ID NO: 29 and a light chain comprising the amino acid sequence of SEQ ID NO: 30, (b) a heavy chain comprising the amino acid sequence of SEQ ID NO: 31 and a light chain comprising the amino acid sequence of SEQ ID NO: 32, (c) a heavy chain comprising the amino acid sequence of SEQ ID NO: 33 and a light chain comprising the amino acid sequence of SEQ ID NO: 34, (d) a heavy chain comprising the amino acid sequence of SEQ ID NO: 35 and a light chain comprising the amino acid sequence of SEQ ID NO: 36, or (e) a heavy chain comprising the amino acid sequence of SEQ ID NO: 37 and a light chain comprising the amino acid sequence of SEQ ID NO: 38.

In a preferred embodiment the anti-PD-1 antibody is PD1-1.

In a preferred embodiment the anti-PD-1 antibody is PD1-2.

In a preferred embodiment the anti-PD-1 antibody is PD1-3.

In a preferred embodiment the anti-PD-1 antibody is PD1-4.

In a preferred embodiment the anti-PD-1 antibody is PD1-5.

In one aspect, the invention provides a method of treating and/or preventing a hyperproliferative disease, preferably cancer, comprising administering to a patient in need thereof a therapeutically effective amount of an LRP5/LRP6 antagonist (e.g. any one of LRP5/LRP6#1, LRP5/LRP6#2, LRP5/LRP6#3, LRP5/LRP6#4, LRP5/LRP6#5, LRP5/LRP6#6 as defined by the CDR and/or VHH sequences of Tables 1a, 1b, 1c, 2a, 2b, 2c) and a therapeutically effective amount of an anti-PD-1 antibody (e.g., any one of PD1-1, PD1-2, PD1-3, PD1-4, PD1-5 as defined by the CDR and/or VH/VL sequences of Tables 3 and 4). In preferred embodiments, the LRP5/LRP6 antagonist comprises an amino acid sequence of SEQ ID NO:64, SEQ ID NO:65 or SEQ ID NO:66 and the PD-1 antibody comprises a heavy chain comprising the amino acid sequence of SEQ ID NO: 29 and a light chain comprising the amino acid sequence of SEQ ID NO: 30. In preferred embodiments, the LRP5/LRP6 antagonist comprises an amino acid sequence of SEQ ID NO:64, SEQ ID NO:65 or SEQ ID NO:66 and the PD-1 antibody comprises a heavy chain comprising the amino acid sequence of SEQ ID NO: 31 and a light chain comprising the amino acid sequence of SEQ ID NO: 32. In preferred embodiments, the LRP5/LRP6 antagonist comprises an amino acid sequence of SEQ ID NO:64, SEQ ID NO:65 or SEQ ID NO:66 and the PD-1 antibody comprises a heavy chain comprising the amino acid sequence of SEQ ID NO: 33 and a light chain comprising the amino acid sequence of SEQ ID NO: 34.

In another aspect the invention provides a combination of an LRP5/LRP6 antagonist (e.g. any one of LRP5/LRP6#1, LRP5/LRP6#2, LRP5/LRP6#3, LRP5/LRP6#4, LRP5/LRP6#5, LRP5/LRP6#6 as defined by the CDR and/or VHH sequences of Tables 1a, 1b, 1c, 2a, 2b, 2c) and an anti-PD-1 antibody as described herein (e.g., any one of PD1-1, PD1-2, PD1-3, PD1-4, PD1-5 as defined by the CDR and/or VH/VL sequences of Tables 3 and 4), particularly for use in a method of treating and/or preventing a hyperproliferative disease, preferably cancer, wherein said method comprises that a therapeutically effective amount of the combination is to be administered to a patient in need thereof. In preferred embodiments, the LRP5/LRP6 antagonist comprises an amino acid sequence of SEQ ID NO:64, SEQ ID NO:65 or SEQ ID NO:66 and the PD-1 antibody comprises a heavy chain comprising the amino acid sequence of SEQ ID NO: 29 and a light chain comprising the amino acid sequence of SEQ ID NO: 30. In preferred embodiments, the LRP5/LRP6 antagonist comprises an amino acid sequence of SEQ ID NO:64, SEQ ID NO:65 or SEQ ID NO:66 and the PD-1 antibody comprises a heavy chain comprising the amino acid sequence of SEQ ID NO: 31 and a light chain comprising the amino acid sequence of SEQ ID NO: 32. In preferred embodiments, the LRP5/LRP6 antagonist comprises an amino acid sequence of SEQ ID NO:64, SEQ ID NO:65 or SEQ ID NO:66 and the PD-1 antibody comprises a heavy chain comprising the amino acid sequence of SEQ ID NO: 33 and a light chain comprising the amino acid sequence of SEQ ID NO: 34.

In another aspect the invention refers to an LRP5/LRP6 antagonist (e.g. any one of LRP5/LRP6#1, LRP5/LRP6#2, LRP5/LRP6#3, LRP5/LRP6#4, LRP5/LRP6#5, LRP5/LRP6#6 as defined by the CDR and/or VHH sequences of Tables 1a, 1b, 1c, 2a, 2b, 2c) for use in a method of treating and/or preventing a hyperproliferative disease, preferably cancer, wherein said method comprises that a therapeutically effective amount of the LRP5/LRP6 antagonist in combination with an anti-PD-1 antibody as described herein (e.g., any one of PD1-1, PD1-2, PD1-3, PD1-4, PD1-5 as defined by the CDR and/or VH/VL sequences of Tables 3 and 4) is to be administered to a patient in need thereof. In preferred embodiments, the LRP5/LRP6 antagonist comprises an amino acid sequence of SEQ ID NO:64, SEQ ID NO:65 or SEQ ID NO:66 and the PD-1 antibody comprises a heavy chain comprising the amino acid sequence of SEQ ID NO: 29 and a light chain comprising the amino acid sequence of SEQ ID NO: 30.

In preferred embodiments, the LRP5/LRP6 antagonist comprises an amino acid sequence of SEQ ID NO:64, SEQ ID NO:65 or SEQ ID NO:66 and the PD-1 antibody comprises a heavy chain comprising the amino acid sequence of SEQ ID NO: 31 and a light chain comprising the amino acid sequence of SEQ ID NO: 32. In preferred embodiments, the LRP5/LRP6 antagonist comprises an amino acid sequence of SEQ ID NO:64, SEQ ID NO:65 or SEQ ID NO:66 and the PD-1 antibody comprises a heavy chain comprising the amino acid sequence of SEQ ID NO: 33 and a light chain comprising the amino acid sequence of SEQ ID NO: 34.

In another aspect the invention refers to an anti-PD-1 antibody as described herein (e.g., any one of PD1-1, PD1-2, PD1-3, PD1-4, PD1-5 as defined by the CDR and/or VH/VL sequences of Tables 3 and 4) for use in a method of treating and/or preventing a hyperproliferative disease, preferably cancer, wherein said method comprises that a therapeutically effective amount of the anti-PD-1 antibody in combination with an LRP5/LRP6 antagonist (e.g. any one of LRP5/LRP6#1, LRP5/LRP6#2, LRP5/LRP6#3, LRP5/LRP6#4, LRP5/LRP6#5, LRP5/LRP6#6 as defined by the CDR and/or VHH sequences of Tables 1a, 1b, 1c, 2a, 2b, 2c) is to be administered to a patient in need thereof. In preferred embodiments, the LRP5/LRP6 antagonist comprises an amino acid sequence of SEQ ID NO:64, SEQ ID NO:65 or SEQ ID NO:66 and the PD-1 antibody comprises a heavy chain comprising the amino acid sequence of SEQ ID NO: 29 and a light chain comprising the amino acid sequence of SEQ ID NO: 30. In preferred embodiments, the LRP5/LRP6 antagonist comprises an amino acid sequence of SEQ ID NO:64, SEQ ID NO:65 or SEQ ID NO:66 and the PD-1 antibody comprises a heavy chain comprising the amino acid sequence of SEQ ID NO: 31 and a light chain comprising the amino acid sequence of SEQ ID NO: 32. In preferred embodiments, the LRP5/LRP6 antagonist comprises an amino acid sequence of SEQ ID NO:64, SEQ ID NO:65 or SEQ ID NO:66 and the PD-1 antibody comprises a heavy chain comprising the amino acid sequence of SEQ ID NO: 33 and a light chain comprising the amino acid sequence of SEQ ID NO: 34.

In another aspect the invention refers to a kit comprising in one or more containers
a first pharmaceutical composition or dosage form comprising an LRP5/LRP6 antagonist (e.g. any one of LRP5/LRP6#1, LRP5/LRP6#2, LRP5/LRP6#3, LRP5/LRP6#4, LRP5/LRP6#5, LRP5/LRP6#6 as defined by the CDR and/or VHH sequences of Tables 1a, 1b, 1c, 2a, 2b, 2 c), and, optionally, one or more pharmaceutically acceptable carriers, excipients and/or vehicles, and
a second pharmaceutical composition or dosage form comprising an anti-PD-1 antibody as described herein (e.g., any one of PD1-1, PD1-2, PD1-3, PD1-4, PD1-5 as defined by the CDR and/or VH/VL sequences of Tables 3 and 4), and, optionally, one or more pharmaceutically acceptable carriers, excipients and/or vehicles.
and optionally a package insert comprising printed instructions.

In preferred embodiments of the kits of the invention, the LRP5/LRP6 antagonist comprises an amino acid sequence of SEQ ID NO:64, SEQ ID NO:65 or SEQ ID NO:66 and the PD-1 antibody comprises a heavy chain comprising the amino acid sequence of SEQ ID NO: 29 and a light chain comprising the amino acid sequence of SEQ ID NO: 30. In preferred embodiments of the kits of the invention, the LRP5/LRP6 antagonist comprises an amino acid sequence of SEQ ID NO:64, SEQ ID NO:65 or SEQ ID NO:66 and the PD-1 antibody comprises a heavy chain comprising the amino acid sequence of SEQ ID NO: 31 and a light chain comprising the amino acid sequence of SEQ ID NO: 32. In preferred embodiments of the kits of the invention, the LRP5/LRP6 antagonist comprises an amino acid sequence of SEQ ID NO:64, SEQ ID NO:65 or SEQ ID NO:66 and the PD-1 antibody comprises a heavy chain comprising the amino acid sequence of SEQ ID NO: 33 and a light chain comprising the amino acid sequence of SEQ ID NO: 34.

Preferably, the package insert comprises printed instructions for simultaneous, concurrent, sequential, successive, alternate or separate use in the treatment and/or prevention of a hyperproliferative disease, in particular cancer, as described herein, in a patient in need thereof.

In another aspect the invention refers to the aforementioned kits for use in a method of treating and/or preventing a hyperproliferative disease, preferably cancer, as described herein.

In another aspect the invention refers to a pharmaceutical composition comprising
an LRP5/LRP6 antagonist (e.g. any one of LRP5/LRP6#1, LRP5/LRP6#2, LRP5/LRP6#3, LRP5/LRP6#4, LRP5/LRP6#5, LRP5/LRP6#6 as defined by the CDR and/or VHH sequences of Tables 1a, 1b, 1c, 2a, 2b, 2c),
a anti-PD-1 antibody as described herein (e.g., any one of PD1-1, PD1-2, PD1-3, PD1-4, PD1-5 as defined by the CDR and/or VH/VL sequences of Tables 3 and 4), and,
optionally, one or more pharmaceutically acceptable carriers, excipients and/or vehicles.

In preferred embodiments of the pharmaceutical composition of the invention, the LRP5/LRP6 antagonist comprises an amino acid sequence of SEQ ID NO:64, SEQ ID NO:65 or SEQ ID NO:66 and the PD-1 antibody comprises a heavy chain comprising the amino acid sequence of SEQ ID NO: 29 and a light chain comprising the amino acid sequence of SEQ ID NO: 30. In preferred embodiments of the pharmaceutical composition of the invention, the LRP5/LRP6 antagonist comprises an amino acid sequence of SEQ ID NO:64, SEQ ID NO:65 or SEQ ID NO:66 and the PD-1 antibody comprises a heavy chain comprising the amino acid sequence of SEQ ID NO: 31 and a light chain comprising the amino acid sequence of SEQ ID NO: 32. In preferred embodiments of the pharmaceutical composition of the invention, the LRP5/LRP6 antagonist comprises an amino acid sequence of SEQ ID NO:64, SEQ ID NO:65 or SEQ ID NO:66 and the PD-1 antibody comprises a heavy chain comprising the amino acid sequence of SEQ ID NO: 33 and a light chain comprising the amino acid sequence of SEQ ID NO: 34.

In another aspect the invention refers to the use of an LRP5/LRP6 antagonist (e.g. any one of LRP5/LRP6#1, LRP5/LRP6#2, LRP5/LRP6#3, LRP5/LRP6#4, LRP5/LRP6#5, LRP5/LRP6#6 as defined by the CDR and/or VHH sequences of Tables 1a, 1b, 1c, 2a, 2b, 2c) for preparing a pharmaceutical composition for use in a method of treating and/or preventing a hyperproliferative disease, preferably cancer, as described herein, wherein the LRP5/LRP6 antagonist is to be used in combination with a PD-1 antibody as described herein (e.g., any one of PD1-1, PD1-2, PD1-3, PD1-4, PD1-5 as defined by the CDR and/or VH/VL sequences of Tables 3 and 4). In preferred embodiments, the LRP5/LRP6 antagonist comprises an amino acid sequence of SEQ ID NO:64, SEQ ID NO:65 or SEQ ID NO:66 and the PD-1 antibody comprises a heavy chain comprising the amino acid sequence of SEQ ID NO: 29 and a light chain comprising the amino acid sequence of SEQ ID NO: 30. In preferred embodiments, the LRP5/LRP6 antagonist comprises an amino acid sequence of SEQ ID NO:64, SEQ ID NO:65 or SEQ ID NO:66 and the PD-1 antibody comprises a heavy chain comprising the amino acid sequence of SEQ ID NO: 31 and a light chain comprising the amino acid sequence of SEQ ID NO: 32. In preferred embodiments, the LRP5/LRP6 antagonist comprises an amino acid sequence of SEQ ID NO:64, SEQ ID NO:65 or SEQ ID NO:66 and the PD-1 antibody comprises a heavy chain comprising the amino acid sequence of SEQ ID NO: 33 and a light chain comprising the amino acid sequence of SEQ ID NO: 34.

In another aspect the invention refers to the use of a PD-1 antibody as described herein (e.g., any one of PD1-1, PD1-2, PD1-3, PD1-4, PD1-5 as defined by the CDR and/or VH/VL sequences of Tables 3 and 4) for preparing a pharmaceutical composition for use in a method of treating and/or preventing a hyperproliferative disease, preferably cancer, as described herein, wherein the PD-1 antagonist is to be used in combination with an LRP5/LRP6 antagonist (e.g. any one of LRP5/LRP6#1, LRP5/LRP6#2, LRP5/LRP6#3, LRP5/LRP6#4, LRP5/LRP6#5, LRP5/LRP6#6 as defined by the CDR and/or VHH sequences of Tables 1a, 1 b, 1c, 2a, 2b, 2c). In preferred embodiments, the LRP5/LRP6 antagonist comprises an amino acid sequence of SEQ ID NO:64, SEQ ID NO:65 or SEQ ID NO:66 and the PD-1 antibody comprises a heavy chain comprising the amino acid sequence of SEQ ID NO: 29 and a light chain comprising the amino acid sequence of SEQ ID NO: 30. In preferred embodiments, the LRP5/LRP6 antagonist comprises an amino acid sequence of SEQ ID NO:64, SEQ ID NO:65 or SEQ ID NO:66 and the PD-1 antibody comprises a heavy chain comprising the amino acid sequence of SEQ ID NO: 31 and a light chain comprising the amino acid sequence of SEQ ID NO: 32. In preferred embodiments, the LRP5/LRP6 antagonist comprises an amino acid sequence of SEQ ID NO:64, SEQ ID NO:65 or SEQ ID NO:66 and the PD-1 antibody comprises a heavy chain comprising the amino acid sequence of SEQ ID NO: 33 and a light chain comprising the amino acid sequence of SEQ ID NO: 34.

In another aspect the invention refers to the use of an LRP5/LRP6 antagonist (e.g. any one of LRP5/LRP6#1, LRP5/LRP6#2, LRP5/LRP6#3, LRP5/LRP6#4, LRP5/LRP6#5, LRP5/LRP6#6 as defined by the CDR and/or VHH sequences of Tables 1a, 1b, 1c, 2a, 2b, 2c) and a PD-1 antibody (e.g., any one of PD1-1, PD1-2, PD1-3, PD1-4, PD1-5 as defined by the CDR and/or VH/VL sequences of Tables 3 and 4), for preparing a pharmaceutical composition for use in a method of treating and/or preventing a hyperproliferative disease, preferably cancer, as described herein. In preferred embodiments, the LRP5/LRP6 antagonist comprises an amino acid sequence of SEQ ID NO:64, SEQ ID NO:65 or SEQ ID NO:66 and the PD-1 antibody comprises a heavy chain comprising the amino acid sequence of SEQ ID NO: 29 and a light chain comprising the amino acid sequence of SEQ ID NO: 30. In preferred embodiments, the LRP5/LRP6 antagonist comprises an amino acid sequence of SEQ ID NO:64, SEQ ID NO:65 or SEQ ID NO:66 and the PD-1 antibody comprises a heavy chain comprising the amino acid sequence of SEQ ID NO: 31 and a light chain comprising the amino acid sequence of SEQ ID NO: 32. In preferred embodiments, the LRP5/LRP6 antagonist comprises an amino acid sequence of SEQ ID NO:64, SEQ ID NO:65 or SEQ ID NO:66 and the PD-1 antibody comprises a heavy chain comprising the amino acid sequence of SEQ ID NO: 33 and a light chain comprising the amino acid sequence of SEQ ID NO: 34.

In another aspect, the invention refers to a combination, a pharmaceutical composition or a kit according to the invention, each as described herein, comprising, consisting or consisting essentially of an LRP5/LRP6 antagonist (e.g. any one of LRP5/LRP6#1, LRP5/LRP6#2, LRP5/LRP6#3, LRP5/LRP6#4, LRP5/LRP6#5, LRP5/LRP6#6 as defined by the CDR and/or VHH sequences of Tables 1a, 1b, 1c, 2a, 2b, 2c) and an anti-PD-1 antibody, (e.g., any one of PD1-1, PD1-2, PD1-3, PD1-4, PD1-5 as defined by the CDR and/or VH/VL sequences of Tables 3 and 4), for use in a method of treating and/or preventing a or hyperproliferative disease preferably cancer, as described herein. In preferred embodiments, the LRP5/LRP6 antagonist comprises an amino acid sequence of SEQ ID NO:64, SEQ ID NO:65 or SEQ ID NO:66 and the PD-1 antibody comprises a heavy chain comprising the amino acid sequence of SEQ ID NO: 29 and a light chain comprising the amino acid sequence of SEQ ID NO: 30. In preferred embodiments, the LRP5/LRP6 antagonist comprises an amino acid sequence of SEQ ID NO:64, SEQ ID NO:65 or SEQ ID NO:66 and the PD-1 antibody comprises a heavy chain comprising the amino acid sequence of SEQ ID NO: 31 and a light chain comprising the amino acid sequence of SEQ ID NO: 32. In preferred embodiments, the LRP5/LRP6 antagonist comprises an amino acid sequence of SEQ ID NO:64, SEQ ID NO:65 or SEQ ID NO:66 and the PD-1 antibody comprises a heavy chain comprising the amino acid sequence of SEQ ID NO: 33 and a light chain comprising the amino acid sequence of SEQ ID NO: 34.

The permutation of embodiments in respect of the LRP5/LRP6 antagonist (e.g. any one of LRP5/LRP6#1, LRP5/LRP6#2, LRP5/LRP6#3, LRP5/LRP6#4, LRP5/LRP6#5, LRP5/LRP6#6 with in respect of the PD-1 antagonist PD1-1, PD1-2, PD1-3, PD1-4, PD1-5 results in specific combinations which shall all be deemed to be specifically disclosed and to be embodiments of the invention and of all of its combinations, compositions, kits, methods, uses and compounds for use including methods applying specific administration/dosing regimens as detailed below and/or for treatment of specific cancers as detailed below.

Routes of administration for the LRP5/LRP6 antagonist and/or the anti-PD1 antibody as described herein, include, but are not limited to parenteral (e.g. intramuscular, intraperitoneal, intravenous, transdermal or subcutaneous injection, or implant), oral, enterical, nasal, vaginal, rectal, or topical administration. In a preferred embodiment, the route of administration is intravenous administration, especially intravenous infusion or injection. The compounds of the present invention may be formulated, alone or together, in suitable dosage unit formulations containing conventional non-toxic pharmaceutically acceptable carriers, excipients and/or vehicles appropriate for each route of administration. More preferably, formulations include solid, semi-solid or liquid dosage forms, such as lyophilisation, liquid solutions (e.g. injectable and infusible solutions), dispersions or suspensions, liposomes and suppositories. The preferred mode depends on the intended mode of administration and therapeutic application. Especially preferred embodiments include liquid formulations and lyophilisation. In the case of a lyophilisation, the lyophilisate may be reconstituted in a liquid, preferably water.

Administration of the anti-PD-1 antibody, as described herein may e.g. be by injection (e.g. subcutaneously or intravenously) at a dose of about 0.1 to 30 mg/kg of patient body weight, e.g. about 0.5 to 25 mg/kg of patient body weight, about 1 to 20 mg/kg of patient body weight, about 2 to 5 mg/kg of patient body weight, or about 3 mg/kg of patient body weight.

In some embodiments, the anti-PD-1 antibody is administered at a dose from about 10 to 20 mg/kg of patient body weight every two weeks. The antibody molecule can be administered by intravenous infusion at a rate of more than 20 mg/min, e.g., 20-40 mg/min, and typically greater than or equal to 40 mg/min to reach a dose of about 35 to 440 mg/m², typically about 70 to 310 mg/m², and more typically, about 110 to 130 mg/m². In some embodiments, the infusion rate of about 110 to 130 mg/m² achieves a level of about 3 mg/kg of patient body weight. In other embodiments, the antibody molecule can be administered by intravenous infusion at a rate of less than 10 mg/min, e.g., less than or equal to 5 mg/min to reach a dose of about 1 to 100 mg/m², e.g., about 5 to 50 mg/m², about 7 to 25 mg/m², or, about 10 mg/m². In some embodiments, the antibody is infused over a period of about 30 min.

Preferred dosage regimens for an anti-PD-1 antibody described herein include 1 mg/kg of patient body weight or alternatively 3 mg/kg of patient body weight via intravenous administration, with the antibody being given every three weeks or every four weeks.

The LRP5/LRP6 antagonist described herein or the compositions comprising the same can for example be administered intravenously (i.v.), subcutaneously (s.c.), intramuscularly (i.m.), intraperitoneally (i.p.), transdermally, orally, sublingually (e.g. in the form of a sublingual tablet, spray or drop placed under the tongue and adsorbed through the mucus membranes into the capillary network under the tongue), (intra-) nasally (e.g. in the form of a nasal spray and/or as an aerosol), topically, by means of a suppository, by inhalation, or any other suitable manner in an effective amount or dose.

The LRP5/LRP6 antagonists described herein will generally be administered in an amount between 0.005 and 20.0 mg per kilogram of patient body weight and dose, preferably between 0.05 and 10.0 mg/kg/dose, and more preferably between 0.5 and 10 mg/kg/dose, but can vary, especially, depending on the specific disease, disorder or condition to be treated, the potency of the specific LRP5/LRP6 antagonist to be used, the specific route of administration and the specific pharmaceutical formulation or composition used. Thus, in some cases it may be sufficient to use less than the minimum dose given above, whereas in other cases the upper limit may have to be exceeded. When administering large amounts it may be advisable to divide them up into a number of smaller doses spread over the day.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions.

The LRP5/LRP6 antagonist and the anti-PD1 antibody as described herein may be administered at therapeutically effective amounts in single or divided doses administered at appropriate time intervals. A therapeutically effective amount refers to an amount effective at dosages and for periods of time necessary to achieve the desired therapeutic result and is the minimum amount necessary to prevent, ameliorate, or treat a disease or disorder. A therapeutically effective amount of the compounds described herein may vary according to factors such as the disease state, age, sex, and weight of the individual, and the ability of the compound to elicit a desired response in the individual. A therapeutically effective amount is also one in which any toxic or detrimental effects of the compound is outweighed by the therapeutically beneficial effects. A therapeutically effective dose preferably inhibits a measurable parameter, e.g. a tumor growth rate by at least about 20%, more preferably by at least about 40%, even more preferably by at least about 60%, and still more preferably by at least about 80% relative to untreated subjects or relative to a preceding untreated period of the same subject that is to be treated.

The active compounds may be administered in such doses which are therapeutically effective in monotherapy, or in such doses which are lower or higher than the doses used in monotherapy, but when combined result in a desired (jointly) therapeutically effective amount. This may for example be useful for avoiding, limiting or reducing any unwanted side-effects that are associated with the use of one or more of the substances or principles when they are used in their usual amounts, while still obtaining the desired pharmacological or therapeutic effect.

The amount of the compounds described herein required for use in treatment may be adapted to the particular compound selected, the route of administration, the nature of the condition being treated and the age and condition of the patient and will be ultimately at the discretion of the attendant physician or clinician. Also, the dosage of the compounds described herein may be adapted depending on the target cell, tumor, tissue, graft, or organ.

The desired dose of the LRP5/LRP6 antagonist or anti-PD-1 antibody both as described herein may be administered as a fixed amount per administration or as bolus, to reach a set blood concentration in the patient.

Within this invention it will be appreciated that the LRP5/LRP6 antagonist and the anti-PD1 antibody can be administered formulated either dependently (i.e. mixed together into one composition) or independently (i.e. as separate compositions), wherein such administration provides therapeutically effective levels of the two compounds in the body of the patient. The latter also applies to cocktail therapy, e.g. the administration of three or more active agents. In other words, the LRP5/LRP6 antagonist and the anti-PD1 antibody may be administered either as part of the same pharmaceutical composition/dosage form or, preferably, in separate pharmaceutical compositions/dosage forms. In as far as the administration is in separate pharmaceutical compositions/dosage forms, it is to be understood that according to this invention said administration envisages the simultaneous, concurrent, sequential or alternate administration of the active agents or components.

The term “simultaneous” (also referred to as “concomitant” herein) refers to the administration of both compounds/compositions at substantially the same time.

Concurrent administration includes administering the active agents within the same general time period, for example on the same day(s) but not necessarily at the same time.

Sequential administration includes administration of one agent during a first time period (for example over the course of a few hours, days or a week) using one or more doses, followed by administration of the other agent during a second time period (for example over the course of a few hours, days or a week) using one or more doses. An overlapping schedule may also be employed, which includes administration of the active agents on different days over the treatment period, not necessarily according to a regular sequence. Alternatively, a successive administration is also envisaged, the second administration step is carried out immediately once the administration of the first compounds has been finished. The skilled person knows how to determine the finish of the first administration step, thereby enabling them to identify the suitable time point for initiation the second administration step.

Alternate administration includes administration of one agent during a time period, for example over the course of a few hours, days or a week, followed by administration of the other agent during a subsequent period of time, for example over the course of a few hours, days or a week, and then repeating the pattern for one or more cycles, wherein the overall number of repeats depends on the chosen dosage regimen.

Variations on these general guidelines may also be employed, e.g. according to the agents used and the condition of the subject.

In a preferred embodiment of the invention, in the method according the present invention, the LRP5/LRP6 antagonist and the anti-PD1 antibody each as described herein are administered simultaneously or concurrently (e.g., by intravenous infusion or subcutaneously) during a first period followed by a second period when the anti-PD1 antibody is administered (e.g., by intravenous infusion or subcutaneously) and the LRP5/LRP6 antagonist is not administered. In some embodiments, the first period is 3 or 6 weeks, when the polypeptide capable of specifically binding to LRP5 and LRP6 and the PD1 antibody are administered every three weeks. In some embodiments, the first period is 4 or 8 weeks, when the polypeptide capable of specifically binding to LRP5 and LRP6 and the PD1 antibody are administered every four weeks. It is particularly preferred that this administration schedule is employed with the LRP5/LRP6 antagonist being LRP5/LRP6#1 and the anti-PD-1 antibody being PD1-1, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies. It is particularly preferred that this administration schedule is employed with the LRP5/LRP6 antagonist being LRP5/LRP6#5 and the anti-PD-1 antibody being PD1-1, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies. It is particularly preferred that this administration schedule is employed with the LRP5/LRP6 antagonist being LRP5/LRP6#6 and the anti-PD-1 antibody being PD1-1, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies. It is particularly preferred that this administration schedule is employed with the LRP5/LRP6 antagonist being LRP5/LRP6#1 and the anti-PD-1 antibody being PD1-2, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies. It is particularly preferred that this administration schedule is employed with the LRP5/LRP6 antagonist being LRP5/LRP6#5 and the anti-PD-1 antibody being PD1-2, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies. It is particularly preferred that this administration schedule is employed with the LRP5/LRP6 antagonist being LRP5/LRP6#6 and the anti-PD-1 antibody being PD1-2, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies. It is particularly preferred that this administration schedule is employed with the LRP5/LRP6 antagonist being LRP5/LRP6#1 and the anti-PD-1 antibody being PD1-3, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies. It is particularly preferred that this administration schedule is employed with the LRP5/LRP6 antagonist being LRP5/LRP6#5 and the anti-PD-1 antibody being PD1-3, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies. It is particularly preferred that this administration schedule is employed with the LRP5/LRP6 antagonist being LRP5/LRP6#6 and the anti-PD-1 antibody being PD1-3, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies.

In another preferred embodiment of the invention, the LRP5/LRP6 antagonist and the anti-PD1 antibody as described herein are both administered (simultaneously or concurrently by intravenous infusion or subcutaneously) every three weeks during a first period (of e.g. 3 or 6 weeks) and then the anti-PD1 antibody is administered, e.g., every three weeks during a second period (e.g., by intravenous infusion or subcutaneously). For example, the LRP5/LRP6 antagonist and the anti-PD1 antibody are administered simultaneously or concurrently (e.g., by intravenous infusion or subcutaneously) in (i) week 1 or (ii) in week 1 and week 4, and then the PD1 antibody is administered, e.g., in week 7, 10, and any subsequent third week (week 13, 16, etc) until treatment is terminated. In case of option (i), the PD1 antibody is already administered alone in week 4 (i.e. instead of the combined administration with the LRP5 antagonist as in option (ii)).

It is particularly preferred that this administration schedule is employed with the LRP5/LRP6 antagonist being LRP5/LRP6#1 and the anti-PD-1 antibody being PD1-1, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies. It is particularly preferred that this administration schedule is employed with the LRP5/LRP6 antagonist being LRP5/LRP6#5 and the anti-PD-1 antibody being PD1-1, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies. It is particularly preferred that this administration schedule is employed with the LRP5/LRP6 antagonist being LRP5/LRP6#6 and the anti-PD-1 antibody being PD1-1, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies. It is particularly preferred that this administration schedule is employed with the LRP5/LRP6 antagonist being LRP5/LRP6#1 and the anti-PD-1 antibody being PD1-2, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies. It is particularly preferred that this administration schedule is employed with the LRP5/LRP6 antagonist being LRP5/LRP6#5 and the anti-PD-1 antibody being PD1-2, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies. It is particularly preferred that this administration schedule is employed with the LRP5/LRP6 antagonist being LRP5/LRP6#6 and the anti-PD-1 antibody being PD1-2, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies. It is particularly preferred that this administration schedule is employed with the LRP5/LRP6 antagonist being LRP5/LRP6#1 and the anti-PD-1 antibody being PD1-3, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies. It is particularly preferred that this administration schedule is employed with the LRP5/LRP6 antagonist being LRP5/LRP6#5 and the anti-PD-1 antibody being PD1-3, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies. It is particularly preferred that this administration schedule is employed with the LRP5/LRP6 antagonist being LRP5/LRP6#6 and the anti-PD-1 antibody being PD1-3, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies.

In another preferred embodiment of the invention, the LRP5/LRP6 antagonist and the anti-PD1 antibody as described herein are both administered (simultaneously or concurrently by intravenous infusion or subcutaneously) every four weeks during a first period (of e.g. 4 or 8 weeks) and then the anti-PD1 antibody is administered, e.g., every four weeks, during a second period (e.g., by intravenous infusion or subcutaneously). For example, the LRP5/LRP6 antagonist and the anti-PD1 antibody are administered simultaneously or concurrently (e.g., by intravenous infusion or subcutaneously) in (i) week 1 or (ii) in week 1 and week 5, and then the PD1 antibody is administered, e.g., in week 9, 13, and any subsequent fourth week (week 17, 21, etc) until treatment is terminated. In case of option (i), the PD1 antibody is already administered alone in week 5 (i.e. instead of the combined administration with the LRP5 antagonist as in option (ii)).

It is particularly preferred that this administration schedule is employed with the LRP5/LRP6 antagonist being LRP5/LRP6#1 and the anti-PD-1 antibody being PD1-1, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies. It is particularly preferred that this administration schedule is employed with the LRP5/LRP6 antagonist being LRP5/LRP6#5 and the anti-PD-1 antibody being PD1-1, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies. It is particularly preferred that this administration schedule is employed with the LRP5/LRP6 antagonist being LRP5/LRP6#6 and the anti-PD-1 antibody being PD1-1, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies. It is particularly preferred that this administration schedule is employed with the LRP5/LRP6 antagonist being LRP5/LRP6#1 and the anti-PD-1 antibody being PD1-2, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies. It is particularly preferred that this administration schedule is employed with the LRP5/LRP6 antagonist being LRP5/LRP6#5 and the anti-PD-1 antibody being PD1-2, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies. It is particularly preferred that this administration schedule is employed with the LRP5/LRP6 antagonist being LRP5/LRP6#6 and the anti-PD-1 antibody being PD1-2, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies. It is particularly preferred that this administration schedule is employed with the LRP5/LRP6 antagonist being LRP5/LRP6#1 and the anti-PD-1 antibody being PD1-3, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies. It is particularly preferred that this administration schedule is employed with the LRP5/LRP6 antagonist being LRP5/LRP6#5 and the anti-PD-1 antibody being PD1-3, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies. It is particularly preferred that this administration schedule is employed with the LRP5/LRP6 antagonist being LRP5/LRP6#6 and the anti-PD-1 antibody being PD1-3, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies.

Preferably, the LRP5/LRP6 antagonist as described herein (e.g., at a dose of about 0.5 to 10 mg/kg of patient body weight) and the anti-PD1 antibody as described herein (e.g. at a dose of any one of 2, 3, 4, or 5 mg/kg of patient body weight) are both administered (simultaneously or concurrently by intravenous infusion or subcutaneously) every three or four weeks during a first period (e.g. corresponding to 1 or 2 dosages) and then the anti-PD1 antibody is administered, e.g., every three or four weeks during a second period (e.g., by intravenous infusion or subcutaneously). It is particularly preferred that this administration schedule is employed with the LRP5/LRP6 antagonist being LRP5/LRP6#1 and the anti-PD-1 antibody being PD1-1, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies. It is particularly preferred that this administration schedule is employed with the LRP5/LRP6 antagonist being LRP5/LRP6#5 and the anti-PD-1 antibody being PD1-1, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies. It is particularly preferred that this administration schedule is employed with the LRP5/LRP6 antagonist being LRP5/LRP6#6 and the anti-PD-1 antibody being PD1-1, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies. It is particularly preferred that this administration schedule is employed with the LRP5/LRP6 antagonist being LRP5/LRP6#1 and the anti-PD-1 antibody being PD1-2, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies. It is particularly preferred that this administration schedule is employed with the LRP5/LRP6 antagonist being LRP5/LRP6#5 and the anti-PD-1 antibody being PD1-2, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies. It is particularly preferred that this administration schedule is employed with the LRP5/LRP6 antagonist being LRP5/LRP6#6 and the anti-PD-1 antibody being PD1-2, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies. It is particularly preferred that this administration schedule is employed with the LRP5/LRP6 antagonist being LRP5/LRP6#1 and the anti-PD-1 antibody being PD1-3, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies. It is particularly preferred that this administration schedule is employed with the LRP5/LRP6 antagonist being LRP5/LRP6#5 and the anti-PD-1 antibody being PD1-3, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies. It is particularly preferred that this administration schedule is employed with the LRP5/LRP6 antagonist being LRP5/LRP6#6 and the anti-PD-1 antibody being PD1-3, even more preferably it is employed for the treatment of gastrointestinal cancers, melanoma, bladder cancer or lung cancer (including gastrointestinal cancers, melanomas, bladder cancer and lung cancer that are refractory or resistant to checkpoint inhibitor therapies) or any solid tumor which is refractory or resistant to checkpoint inhibitor therapies.

In some embodiments of the invention, the LRP5/LRP6 antagonist and the anti-PD1 antibody as described herein are both administered (simultaneously or concurrently by intravenous infusion or subcutaneously) every three or four weeks during a first period (e.g. corresponding to 1 or 2 dosages) and then the anti-PD1 antibody is administered weekly, every other week, every three weeks or monthly during a second period (e.g., by intravenous infusion or subcutaneously).

Depending on the disease to be treated, the combination therapy as defined herein may be used on its own or in further combination with one or more additional therapeutic agents, in particular selected from chemotherapeutic agents or therapeutically active compounds that inhibit angiogenesis, signal transduction pathways or mitotic checkpoints in cancer cells.

The additional therapeutic agent may be administered simultaneously with, optionally as a component of the same pharmaceutical preparation, or before or after administration of the LRP5/LRP6 antagonist and/or the PD1 antibody.

This/these additional therapeutic agent(s) may (each) be selected from the following (without being limited thereto):
an immunotherapeutic agent, such as modulators of the following checkpoint inhibitors: TIM3, PD-L1, PD-L2, CTLA-4, VISTA, BTLA, TIGIT, CD160, LAIR1, 2B4, CEACAM;
a cancer vaccine;
a DNA damaging agent;
an inhibitor of angiogenesis;
an inhibitor of signal transduction pathways;
an inhibitor of mitotic checkpoints; and

hormones, hormone analogues and antihormones (e.g. tamoxifen, toremifene, raloxifene, fulvestrant, megestrol acetate, flutamide, nilutamide, bicalutamide, aminoglutethimide, cyproterone acetate, finasteride, buserelin acetate, fludrocortisone, fluoxymesterone, medroxyprogesterone, octreotide), aromatase inhibitors (e.g. anastrozole, letrozole, liarozole, vorozole, exemestane, atamestane), LHRH agonists and antagonists (e.g. goserelin acetate, luprolide), inhibitors of growth factors (growth factors such as for example “platelet derived growth factor (PDGF)”, “fibroblast growth factor (FGF)”, “vascular endothelial growth factor (VEGF)”, “epidermal growth factor (EGF)”, “insuline-like growth factors (IGF)”, “human epidermal growth factor (HER, e.g. HER2, HER3, HER4)” and “hepatocyte growth factor (HGF)”), inhibitors are for example “growth factor” antibodies, “growth factor receptor” antibodies and tyrosine kinase inhibitors, such as for example cetuximab, gefitinib, imatinib, lapatinib, bosutinib and trastuzumab); antimetabolites (e.g. antifolates such as methotrexate, raltitrexed, pyrimidine analogues such as 5-fluorouracil (5-FU), capecitabine and gemcitabine, purine and adenosine analogues such as mercaptopurine, thioguanine, cladribine and pentostatin, cytarabine (ara C), fludarabine); antitumour antibiotics (e.g. anthracyclins such as doxorubicin, doxil (pegylated liposomal doxorubicin hydrochloride, myocet (non-pegylated liposomal doxorubicin), daunorubicin, epirubicin and idarubicin, mitomycin-C, bleomycin, dactinomycin, plicamycin, streptozocin); platinum derivatives (e.g. cisplatin, oxaliplatin, carboplatin); alkylation agents (e.g. estramustin, meclorethamine, melphalan, chlorambucil, busulphan, dacarbazin, cyclophosphamide, ifosfamide, temozolomide, nitrosoureas such as for example carmustin and lomustin, thiotepa); antimitotic agents (e.g. Vinca alkaloids such as for example vinblastine, vindesin, vinorelbin and vincristine; and taxanes such as paclitaxel, docetaxel); angiogenesis inhibitors (e.g. tasquinimod), tubuline inhibitors; DNA synthesis inhibitors (e.g. sapacitabine), PARP inhibitors, topoisomerase inhibitors (e.g. epipodophyllotoxins such as for example etoposide and etopophos, teniposide, amsacrin, topotecan, irinotecan, mitoxantrone), serine/threonine kinase inhibitors (e.g. PDK 1 inhibitors, Raf inhibitors, A-Raf inhibitros, B-Raf inhibitors, C-Raf inhibitors, mTOR inhibitors, mTORC1/2 inhibitors, PI3K inhibitors, PI3Kα inhibitors, dual mTOR/PI3K inhibitors, STK 33 inhibitors, AKT inhibitors, PLK 1 inhibitors, inhibitors of CDKs, Aurora kinase inhibitors), tyrosine kinase inhibitors (e.g. PTK2/FAK inhibitors), protein protein interaction inhibitors (e.g. IAP activator, Mcl-1, MDM2/MDMX), MEK inhibitors (e.g. pimasertib), ERK inhibitors, FLT3 inhibitors (e.g. quizartinib), BRD4 inhibitors, IGF-1R inhibitors, TRAILR2 agonists, Bcl-xL inhibitors, Bcl-2 inhibitors (e.g. venetoclax), Bcl-2/Bcl-xL inhibitors, ErbB receptor inhibitors, BCR-ABL inhibitors, ABL inhibitors, Src inhibitors, rapamycin analogs (e.g. everolimus, temsirolimus, ridaforolimus, sirolimus), androgen synthesis inhibitors (e.g. abiraterone, TAK-700), androgen receptor inhibitors (e.g. enzalutamide, ARN-509), immunotherapy (e.g. sipuleucel-T), DNMT inhibitors (e.g. SGI 110, temozolomide, vosaroxin), HDAC inhibitors (e.g. vorinostat, entinostat, pracinostat, panobinostat), ANG1/2 inhibitors (e.g. trebananib), CYP17 inhibitors (e.g. galeterone), radiopharmaceuticals (e.g. radium-223, alpharadin), immunotherapeutic agents (e.g. poxvirus-based vaccine, ipilimumab, immune checkpoint inhibitors) and various chemotherapeutic agents such as amifostin, anagrelid, clodronat, filgrastin, interferon, interferon alpha, leucovorin, rituximab, procarbazine, levamisole, mesna, mitotane, pamidronate and porfimer;

2-chlorodesoxyadenosine, 2-fluorodesoxycytidine, 2-methoxyoestradiol, 2C4, 3-alethine, 131-I-TM-601, 3CPA, 7-ethyl-10-hydroxycamptothecin, 16-aza-epothilone B, ABT-199, ABT-263/navitoclax, ABT-737, A 105972, A 204197, aldesleukin, alisertib/MLN8237, alitretinoin, allovectin-7, altretamine, alvocidib, amonafide, anthrapyrazole, AG-2037, AP-5280, apaziquone, apomine, aranose, arglabin, arzoxifene, atamestane, atrasentan, auristatin PE, AVLB, AZ10992, ABX-EGF, AMG-479 (ganitumab), AMG-232, AMG-511, AMG 2520765, AMG 2112819, ARRY 162, ARRY 438162, ARRY-300, ARRY-142886/AZD-6244 (selumetinib), ARRY-704/AZD-8330, ATSP-7041, AR-12, AR-42, AS-703988, AXL-1717, AZD-1480, AZD-4547, AZD-8055, AZD-5363, AZD-6244, AZD-7762, ARQ-736, ARQ 680, AS-703026 (primasertib), avastin, AZD-2014, azacitidine (5-aza), azaepothilone B, azonafide, barasertib/AZD1152, BAY-43-9006, BAY 80-6946, BBR-3464, BBR-3576, bevacizumab, BEZ-235/dactolisib, biricodar dicitrate, birinapant, BCX-1777, BKM-120/buparlisib, bleocin, BLP-25, BMS-184476, BMS-247550, BMS-188797, BMS-275291, BMS-663513, BMS-754807, BNP-1350, BNP-7787, BIBW 2992/afatinib, BIBF 1120/nintedanib, BI 836845, BI 2536, BI 6727/volasertib, BI 836845, BI 847325, BI 853520, BIIB-022, bleomycinic acid, bleomycin A, bleomycin B, brivanib, bryostatin-1, bortezomib, brostallicin, busulphan, BYL-719/alpelisib, CA-4 prodrug, CA-4, cabazitaxel, cabozantinib, CapCell, calcitriol, canertinib, canfosfamide, capecitabine, carboxyphthalatoplatin, CCI-779, CC-115, CC-223, CEP-701, CEP-751, CBT-1 cefixime, ceflatonin, ceftriaxone, celecoxib, celmoleukin, cemadotin, CGM-097, CH4987655/RO-4987655, chlorotrianisene, cilengitide, ciclosporin, CD20 antibodies, CDA-II, CDC-394, CKD-602, CKI-27, clofarabine, colchicin, combretastatin A4, COT inhibitors, CHS-828, CH-5132799, CLL-Thera, CMT-3 cryptophycin 52, CPI-613, CTP-37, CTLA-4 monoclonal antibodies (e.g. ipilimumab), CP-461, crizotinib, CV-247, cyanomorpholinodoxorubicin, cytarabine, D 24851, dasatinib, decitabine, deoxorubicin, deoxyrubicin, deoxycoformycin, depsipeptide, desoxyepothilone B, dexamethasone, dexrazoxanet, diethylstilbestrol, diflomotecan, didox, DMDC, dolastatin 10, doranidazole, DS-7423, DS-3032, E7010, E-6201, edatrexat, edotreotide, efaproxiral, eflornithine, EGFR inhibitors, EKB-569, EKB-509, enzastaurin, elesclomol, elsamitrucin, epothilone B, epratuzumab, EPZ-004777, ER-86526, erlotinib, ET-18-OCH3, ethynylcytidine, ethynyloestradiol, exatecan, exatecan mesylate, exemestane, exisulind, fenretinide, figitumumab, floxuridine, folic acid, FOLFOX, FOLFOX4, FOLFIRI, formestane, fostamatinib, fotemustine, galarubicin, gallium maltolate, ganetespib, gefinitib, gemtuzumab, gemtuzumab ozogamicin, gimatecan, glufosfamide, GCS-IOO, GDC-0623, GDC-0941 (pictrelisib), GDC-0980, GDC-0032, GDC-0068, GDC-0349, GDC-0879, G17DT immunogen, GMK, GMX-1778, GPX-100, gp100-peptide vaccines, GSK-5126766, GSK-690693, GSK-1120212 (trametinib), GSK-1995010, GSK-2118436 (dabrafenib), GSK-2126458, GSK-2132231A, GSK-2334470, GSK-2110183, GSK-2141795, GSK-2636771, GSK-525762A/I-BET-762, GW2016, granisetron, herceptine, hexamethylmelamine, histamine, homoharringtonine, hyaluronic acid, hydroxyurea, hydroxyprogesterone caproate, HDM-201, ibandronate, ibritumomab, ibrutinib/PCI-32765, idasanutlin, idatrexate, idelalisib/CAL-101, idenestrol, IDN-5109, IGF-1R inhibitors, IMC-1C11, IMC-A12 (cixutumumab), immunol, indisulam, interferon alpha-2a, interferon alpha-2b, pegylated interferon alpha-2b, interleukin-2, INK-1117, INK-128, INSM-18, ionafarnib, iproplatin, irofulven, isohomohalichondrin-B, isoflavone, isotretinoin, ixabepilone, JRX-2, JSF-154, JQ-1, J-107088, conjugated oestrogens, kahalid F, ketoconazole, KW-2170, KW-2450, KU-55933, LCL-161, lobaplatin, leflunomide, lenalidomide, lenograstim, leuprolide, leuporelin, lexidronam, LGD-1550, linezolid, lovastatin, lutetium texaphyrin, lometrexol, lonidamine, losoxantrone, LU 223651, lurbinectedin, lurtotecan, LY-S6AKT1, LY-2780301, LY-2109761/galunisertib, mafosfamide, marimastat, masoprocol, mechloroethamine, MEK inhibitors, MEK-162, methyltestosteron, methylprednisolone, MEDI-573, MEN-10755, MDX-H210, MDX-447, MDX-1379, MGV, midostaurin, minodronic acid, mitomycin, mivobulin, MK-2206, MK-0646 (dalotuzumab), MLN518, MLN-0128, MLN-2480, motexafin gadolinium, MS-209, MS-275, MX6, neridronate, neratinib, Nexavar, neovastat, nilotinib, nimesulide, nitroglycerin, nolatrexed, norelin, N-acetylcysteine, NU-7441 06-benzylguanine, oblimersen, omeprazole, olaparib, oncophage, oncoVEX^{GM-CSF}, ormiplatin, ortataxel, OX44 antibodies, OSI-027, OSI-906 (linsitinib), 4-1BB antibodies, oxantrazole, oestrogen, onapristone, palbociclib/PD-0332991, panitumumab, panobinostat, patupilone, pazopanib, pegfilgrastim, PCK-3145, pegfilgrastim, PBI-1402, PBI-05204, PD0325901, PD-1 and PD-L1 antibodies (e.g. pembrolizumab, nivolumab, pidilizumab, MEDI-4736/durvalumab, RG-7446/atezolizumab), PD-616, PEG-paclitaxel, albumin-stabilized paclitaxel, PEP-005, PF-05197281, PF-05212384, PF-04691502, PF-3758309, PHA-665752, PHT-427, P-04, PKC412, P54, PI-88, pelitinib, pemetrexed, pentrix, perifosine, perillylalcohol, pertuzumab, pevonedistat, PI3K inhibitors, PI3K/mTOR inhibitors, PG-TXL, PG2, PLX-4032/RO-5185426 (vemurafenib), PLX-3603/RO-5212054, PT-100, PWT-33597, PX-866, picoplatin, pivaloyloxymethylbutyrate, pixantrone, phenoxodiol O, PKI166, plevitrexed, plicamycin, polyprenic acid, ponatinib, porfiromycin, posaconazole, prednisone, prednisolone, PRT-062607, quinamed, quinupristin, quizartinib/AC220, R115777, RAF-265, ramosetron, ranpirnase, RDEA-119/BAY 869766, RDEA-436, rebeccamycin analogues, receptor tyrosine kinase (RTK) inhibitors, revimid, RG-7167, RG-7112, RG-7304, RG-7421, RG-7321, RG-7356, RG 7440, RG-7775, rhizoxin, rhu-MAb, rigosertib rinfabate, risedronate, rituximab, robatumumab, rofecoxib, romidepsin, RO-4929097, RO-31-7453, RO-5126766, RO-5068760, RPR 109881A, rubidazone, rubitecan, R-flurbiprofen, RX-0201, ruxolitinib, S-9788, sabarubicin, SAHA, sapacitabine, SAR-405838, sargramostim, satraplatin, SB-408075, SB-431542, Se-015/Ve-015, SU5416, SU6668, SDX-101, selinexor, semustin, seocalcitol, SM-11355, SN-38, SN-4071, SR-27897, SR-31747, SR-13668, SRL-172, sorafenib, spiroplatin, squalamine, STF-31, suberanilohydroxamic acid, sutent, T 900607, T 138067, TAE-684, TAK-733, TAS-103, tacedinaline, talaporfin, tanespimycin, Tarceva, tariquitar, tasisulam, taxotere, taxoprexin, tazarotene, tegafur, temozolamide, tesmilifene, testosterone, testosterone propionate, tesmilifene, tetraplatin, tetrodotoxin, tezacitabine, thalidomide, theralux, therarubicin, thymalfasin, thymectacin, tiazofurin, tipifarnib, tirapazamine, tocladesine, tomudex, toremofin, tosedostat. trabectedin, TransMID-107, transretinic acid, traszutumab, tremelimumab, tretinoin, triacetyluridine, triapine, triciribine, trimetrexate, TLK-286TXD 258, tykerb/tyverb, urocidin, valproic acid, valrubicin, vandetanib, vatalanib, vincristine, vinflunine, virulizin, vismodegib, vosaroxin, WX-UK1, WX-554, vectibix, XAV-939, xeloda, XELOX, XL-147, XL-228, XL-281, XL-518/R-7420/GDC-0973, XL-765, YM-511, YM-598, ZD-4190, ZD-6474, ZD-4054, ZD-0473, ZD-6126, ZD-9331, ZD1839, ZSTK-474, zoledronat and zosuquidar.

In some embodiments, the combination therapy as described involves the LRP5/LRP6 antagonist and the anti-PD-1 antibody as described herein without any additional chemotherapeutic agent.

Hyperproliferative Diseases/Cancers

The combinations, compositions, kits, uses, methods and compounds for use according to the present invention (including all embodiments) are useful for the treatment and/or prevention of hyperproliferative disorders, in particular cancer.

In certain embodiments the combinations, compositions, kits, uses, methods and compounds for use according to the present invention (including all embodiments) are useful for the treatment of hyperproliferative disorders, in particular cancer.

As used herein, “hyperproliferative disease” refers to conditions wherein cell growth is increased over normal levels. For example, hyperproliferative diseases or disorders include malignant diseases (e.g. esophageal cancer, colon cancer, biliary cancer) and non-malignant diseases (e.g. atherosclerosis, benign hyperplasia, benign prostatic hypertrophy).

In preferred embodiments, the hyperproliferative disorder is cancer. In a preferred embodiment, said cancer is characterized in that it harbors a mutated/inactivated RNF43 or (an) activating R-Spondin fusion transcript(s).

Cancers are classified in two ways: by the type of tissue in which the cancer originates (histological type) and by primary site, or the location in the body, where the cancer first developed. The most common sites in which cancer develops include the skin, lung, breast, prostate, colon and rectum, cervix and uterus as well as the hematological compartment

The combinations, compositions, kits, uses, methods and compounds for use according to the invention (including all embodiments) may be useful in the treatment of a variety of hyperproliferative disorders, in particular cancers, including, for example, but not limited to the following:
gastrointestinal cancers such as esophageal cancer (e.g., gastroesophageal junction cancer), stomach (gastric) cancer, hepatocellularcarcinoma, biliary tract cancer (e.g., cholangiocarcinoma), gallbladder cancer, pancreatic cancer or colorectal cancer (CRC);
melanoma;
bladder cancer; and
lung cancer (e.g. NSCLC).

In some embodiments of the invention, the combinations, compositions, kits, uses, methods and compounds for use according to the invention (including all embodiments) are used to treat gastrointestinal cancers, preferably esophageal cancer (e.g., gastroesophageal junction cancer), stomach (gastric) cancer, hepatocellularcarcinoma, biliary tract cancer (e.g., cholangiocarcinoma), gallbladder cancer, pancreatic cancer or colorectal cancer (CRC). It is particularly preferred that these cancers are treated with LRP5/LRP6#1 as the LRP5/LRP6 antagonist and PD1-1 as the anti-PD-1 antibody. It is particularly preferred that these cancers are treated with LRP5/LRP6#5 as the LRP5/LRP6 antagonist and PD1-1 as the anti-PD-1 antibody. It is particularly preferred that these cancers are treated with LRP5/LRP6#6 as the LRP5/LRP6 antagonist and PD1-1 as the anti-PD-1 antibody. It is particularly preferred that these cancers are treated with LRP5/LRP6#1 as the LRP5/LRP6 antagonist and PD1-2 as the anti-PD-1 antibody. It is particularly preferred that these cancers are treated with LRP5/LRP6#5 as the LRP5/LRP6 antagonist and PD1-2 as the anti-PD-1 antibody. It is particularly preferred that these cancers are treated with LRP5/LRP6#6 as the LRP5/LRP6 antagonist and PD1-2 as the anti-PD-1 antibody. It is particularly preferred that these cancers are treated with LRP5/LRP6#1 as the LRP5/LRP6 antagonist and PD1-3 as the anti-PD-1 antibody. It is particularly preferred that these cancers are treated with LRP5/LRP6#5 as the LRP5/LRP6 antagonist and PD1-3 as the anti-PD-1 antibody. It is particularly preferred that these cancers are treated with LRP5/LRP6#6 as the LRP5/LRP6 antagonist and PD1-3 as the anti-PD-1 antibody.

In some embodiments of the invention, the combinations, compositions, kits, uses, methods and compounds for use according to the invention (including all embodiments) are used in the treatment of melanoma. It is particularly preferred that these cancers are treated with LRP5/LRP6#1 as the LRP5/LRP6 antagonist and PD1-1 as the anti-PD-1 antibody. It is particularly preferred that these cancers are treated with LRP5/LRP6#5 as the LRP5/LRP6 antagonist and PD1-1 as the anti-PD-1 antibody. It is particularly preferred that these cancers are treated with LRP5/LRP6#6 as the LRP5/LRP6 antagonist and PD1-1 as the anti-PD-1 antibody. It is particularly preferred that these cancers are treated with LRP5/LRP6#1 as the LRP5/LRP6 antagonist and PD1-2 as the anti-PD-1 antibody. It is particularly preferred that these cancers are treated with LRP5/LRP6#5 as the LRP5/LRP6 antagonist and PD1-2 as the anti-PD-1 antibody. It is particularly preferred that these cancers are treated with LRP5/LRP6#6 as the LRP5/LRP6 antagonist and PD1-2 as the anti-PD-1 antibody. It is particularly preferred that these cancers are treated with LRP5/LRP6#1 as the LRP5/LRP6 antagonist and PD1-3 as the anti-PD-1 antibody. It is particularly preferred that these cancers are treated with LRP5/LRP6#5 as the LRP5/LRP6 antagonist and PD1-3 as the anti-PD-1 antibody. It is particularly preferred that these cancers are treated with LRP5/LRP6#6 as the LRP5/LRP6 antagonist and PD1-3 as the anti-PD-1 antibody.

In some embodiments of the invention, the combinations, compositions, kits, uses, methods and compounds for use according to the invention (including all embodiments) are used in the treatment of bladder cancer. It is particularly preferred that these cancers are treated with LRP5/LRP6#1 as the LRP5/LRP6 antagonist and PD1-1 as the anti-PD-1 antibody. It is particularly preferred that these cancers are treated with LRP5/LRP6#5 as the LRP5/LRP6 antagonist and PD1-1 as the anti-PD-1 antibody. It is particularly preferred that these cancers are treated with LRP5/LRP6#6 as the LRP5/LRP6 antagonist and PD1-1 as the anti-PD-1 antibody. It is particularly preferred that these cancers are treated with LRP5/LRP6#1 as the LRP5/LRP6 antagonist and PD1-2 as the anti-PD-1 antibody. It is particularly preferred that these cancers are treated with LRP5/LRP6#5 as the LRP5/LRP6 antagonist and PD1-2 as the anti-PD-1 antibody. It is particularly preferred that these cancers are treated with LRP5/LRP6#6 as the LRP5/LRP6 antagonist and PD1-2 as the anti-PD-1 antibody. It is particularly preferred that these cancers are treated with LRP5/LRP6#1 as the LRP5/LRP6 antagonist and PD1-3 as the anti-PD-1 antibody. It is particularly preferred that these cancers are treated with LRP5/LRP6#5 as the LRP5/LRP6 antagonist and PD1-3 as the anti-PD-1 antibody. It is particularly preferred that these cancers are treated with LRP5/LRP6#6 as the LRP5/LRP6 antagonist and PD1-3 as the anti-PD-1 antibody.

In some embodiment of the invention, the combinations, compositions, kits, uses, methods and compounds for use according to the invention (including all embodiments) are used in the treatment of lung cancer (e.g. Non-small-cell lung carcinoma NSCLC). It is particularly preferred that these cancers are treated with LRP5/LRP6#1 as the LRP5/LRP6 antagonist and PD1-1 as the anti-PD-1 antibody. It is particularly preferred that these cancers are treated with LRP5/LRP6#5 as the LRP5/LRP6 antagonist and PD1-1 as the anti-PD-1 antibody. It is particularly preferred that these cancers are treated with LRP5/LRP6#6 as the LRP5/LRP6 antagonist and PD1-1 as the anti-PD-1 antibody. It is particularly preferred that these cancers are treated with LRP5/LRP6#1 as the LRP5/LRP6 antagonist and PD1-2 as the anti-PD-1 antibody. It is particularly preferred that these cancers are treated with LRP5/LRP6#5 as the LRP5/LRP6 antagonist and PD1-2 as the anti-PD-1 antibody. It is particularly preferred that these cancers are treated with LRP5/LRP6#6 as the LRP5/LRP6 antagonist and PD1-2 as the anti-PD-1 antibody. It is particularly preferred that these cancers are treated with LRP5/LRP6#1 as the LRP5/LRP6 antagonist and PD1-3 as the anti-PD-1 antibody. It is particularly preferred that these cancers are treated with LRP5/LRP6#5 as the LRP5/LRP6 antagonist and PD1-3 as the anti-PD-1 antibody. It is particularly preferred that these cancers are treated with LRP5/LRP6#6 as the LRP5/LRP6 antagonist and PD1-3 as the anti-PD-1 antibody.

In a further embodiment of the invention, the combinations, compositions, kits, uses, methods and compounds for use according to the invention (including all embodiments) are used in the treatment of cancer patients (e.g. patients suffering from (i) a gastrointestinal cancer such as esophageal cancer gastric cancer, hepatocellularcarcinoma, biliary tract cancer gallbladder cancer, pancreatic cancer or colorectal cancer, (ii) melanoma, (iii) bladder cancer or (iv) lung cancer) who are treatment naïve in respect of treatment with a checkpoint inhibitor or immunomodulator, i.e., e.g., patients who are treatment naïve in respect of treatment with an anti-PD-1 antibody). In one embodiment, said cancer is characterized in that it harbors a mutated/inactivated RNF43 or (an) activating R-Spondin fusion transcript(s). It is particularly preferred that these cancers are treated with LRP5/LRP6#1 as the LRP5/LRP6 antagonist and PD1-1 as the anti-PD-1 antibody. It is particularly preferred that these cancers are treated with LRP5/LRP6#5 as the LRP5/LRP6 antagonist and PD1-1 as the anti-PD-1 antibody. It is particularly preferred that these cancers are treated with LRP5/LRP6#6 as the LRP5/LRP6 antagonist and PD1-1 as the anti-PD-1 antibody. It is particularly preferred that these cancers are treated with LRP5/LRP6#1 as the LRP5/LRP6 antagonist and PD1-2 as the anti-PD-1 antibody. It is particularly preferred that these cancers are treated with LRP5/LRP6#5 as the LRP5/LRP6 antagonist and PD1-2 as the anti-PD-1 antibody. It is particularly preferred that these cancers are treated with LRP5/LRP6#6 as the LRP5/LRP6 antagonist and PD1-2 as the anti-PD-1 antibody. It is particularly preferred that these cancers are treated with LRP5/LRP6#1 as the LRP5/LRP6 antagonist and PD1-3 as the anti-PD-1 antibody. It is particularly preferred that these cancers are treated with LRP5/LRP6#5 as the LRP5/LRP6 antagonist and PD1-3 as the anti-PD-1 antibody. It is particularly preferred that these cancers are treated with LRP5/LRP6#6 as the LRP5/LRP6 antagonist and PD1-3 as the anti-PD-1 antibody.

In a further embodiment of the invention, the combinations, compositions, kits, uses, methods and compounds for use according to the invention (including all embodiments) are used in the treatment of cancer patients (e.g. patients suffering from (i) a gastrointestinal cancer such as esophageal cancer gastric cancer, hepatocellularcarcinoma, biliary tract cancer gallbladder cancer, pancreatic cancer or colorectal cancer, (ii) melanoma, (iii) bladder cancer or (iv) lung cancer) who relapsed during, subsequently or after treatment with a checkpoint inhibitor or immunomodulator, i.e., e.g., patients who relapsed during, subsequently or after treatment with a PD-1 antagonist such as an anti-PD-1 antibody. In one embodiment, said cancer is characterized in that it harbors a mutated/inactivated RNF43 or (an) activating R-Spondin fusion transcript(s). It is particularly preferred that these cancers are treated with LRP5/LRP6#1 as the LRP5/LRP6 antagonist and PD1-1 as the anti-PD-1 antibody. It is particularly preferred that these cancers are treated with LRP5/LRP6#5 as the LRP5/LRP6 antagonist and PD1-1 as the anti-PD-1 antibody. It is particularly preferred that these cancers are treated with LRP5/LRP6#6 as the LRP5/LRP6 antagonist and PD1-1 as the anti-PD-1 antibody. It is particularly preferred that these cancers are treated with LRP5/LRP6#1 as the LRP5/LRP6 antagonist and PD1-2 as the anti-PD-1 antibody. It is particularly preferred that these cancers are treated with LRP5/LRP6#5 as the LRP5/LRP6 antagonist and PD1-2 as the anti-PD-1 antibody. It is particularly preferred that these cancers are treated with LRP5/LRP6#6 as the LRP5/LRP6 antagonist and PD1-2 as the anti-PD-1 antibody. It is particularly preferred that these cancers are treated with LRP5/LRP6#1 as the LRP5/LRP6 antagonist and PD1-3 as the anti-PD-1 antibody. It is particularly preferred that these cancers are treated with LRP5/LRP6#5 as the LRP5/LRP6 antagonist and PD1-3 as the anti-PD-1 antibody. It is particularly preferred that these cancers are treated with LRP5/LRP6#6 as the LRP5/LRP6 antagonist and PD1-3 as the anti-PD-1 antibody.

The therapeutic applicability of the combination therapy according to this invention may include first line, second line, third line or further lines of treatment of patients (e.g. patients suffering from (i) a gastrointestinal cancer such as esophageal cancer, gastric cancer, hepatocellularcarcinoma, biliary tract cancer gallbladder cancer, pancreatic cancer or colorectal cancer, (ii) melanoma, (iii) bladder cancer or (iv) lung cancer). The cancer may be metastatic, recurrent, relapsed, resistant or refractory to one or more anti-cancer treatments. Thus, the patients may be treatment naïve, or may have received one or more previous anti-cancer therapies, which have not completely cured the disease.

Patients with relapse and/or with resistance to one or more anti-cancer agents (e.g. the single components of the combination, or standard chemotherapeutics) are also amenable for combined treatment according to this invention, e.g. for second or third line treatment cycles (optionally in further combination with one or more other anti-cancer agents), e.g. as add-on combination or as replacement treatment.

Accordingly, some of the disclosed combination therapies of this invention are effective at treating subjects (e.g. patients suffering from (i) a gastrointestinal cancer such as esophageal cancer, gastric cancer, hepatocellularcarcinoma, biliary tract cancer gallbladder cancer, pancreatic cancer or colorectal cancer, (ii) melanoma, (iii) bladder cancer or (iv) lung cancer) whose cancer has relapsed, or whose cancer has become drug resistant or multi-drug resistant, or whose cancer has failed one, two or more lines of mono- or combination therapy with one or more anti-cancer agents (e.g. the single components of the combination, or standard chemotherapeutics).

A cancer which initially responded to an anti-cancer drug can relapse and it can become resistant to the anti-cancer drug when the anti-cancer drug is no longer effective in treating the subject with the cancer, e.g. despite the administration of increased dosages of the anti-cancer drug. Cancers that have developed resistance to two or more anti-cancer drugs are said to be multi-drug resistant.

In preferred embodiments the combinations, compositions, kits, uses, methods and compounds for use according to the invention (including all embodiments) are used in the treatment of cancer patients (e.g. patients suffering from (i) a gastrointestinal cancer such as esophageal cancer, gastric cancer, hepatocellularcarcinoma, biliary tract cancer gallbladder cancer, pancreatic cancer or colorectal cancer, (ii) melanoma, (iii) bladder cancer or (iv) lung cancer) who have been previously treated with one or more immune checkpoint inhibitor and/or immuno modulator, e.g. one or more PD-1 antagonist(s) such as an anti-PD1 antibody. In one embodiment, said cancer is characterized in that it harbors a mutated/inactivated RNF43 or (an) activating R-Spondin fusion transcript(s). It is particularly preferred that these cancers are treated with LRP5/LRP6#1 as the LRP5/LRP6 antagonist and PD1-1 as the anti-PD-1 antibody. It is particularly preferred that these cancers are treated with LRP5/LRP6#5 as the LRP5/LRP6 antagonist and PD1-1 as the anti-PD-1 antibody. It is particularly preferred that these cancers are treated with LRP5/LRP6#6 as the LRP5/LRP6 antagonist and PD1-1 as the anti-PD-1 antibody. It is particularly preferred that these cancers are treated with LRP5/LRP6#1 as the LRP5/LRP6 antagonist and PD1-2 as the anti-PD-1 antibody. It is particularly preferred that these cancers are treated with LRP5/LRP6#5 as the LRP5/LRP6 antagonist and PD1-2 as the anti-PD-1 antibody. It is particularly preferred that these cancers are treated with LRP5/LRP6#6 as the LRP5/LRP6 antagonist and PD1-2 as the anti-PD-1 antibody. It is particularly preferred that these cancers are treated with LRP5/LRP6#1 as the LRP5/LRP6 antagonist and PD1-3 as the anti-PD-1 antibody. It is particularly preferred that these cancers are treated with LRP5/LRP6#5 as the LRP5/LRP6 antagonist and PD1-3 as the anti-PD-1 antibody. It is particularly preferred that these cancers are treated with LRP5/LRP6#6 as the LRP5/LRP6 antagonist and PD1-3 as the anti-PD-1 antibody.

In a further preferred embodiment, the combinations, compositions, kits, uses, methods and compounds for use according to the invention (including all embodiments) are used in the treatment of cancer patients (e.g. patients suffering from (i) a gastrointestinal cancer such as esophageal cancer, gastric cancer, hepatocellularcarcinoma, biliary tract cancer gallbladder cancer, pancreatic cancer or colorectal cancer, (ii) melanoma, (iii) bladder cancer or (iv) lung cancer) who are refractory or resistant to checkpoint inhibitor therapies (e.g. to treatment with one or more immune checkpoint inhibitor and/or immuno modulators, e.g. one or more PD-1 antagonist(s) such as an anti-PD1 antibody). In one embodiment, said cancer is characterized in that it harbors a mutated/inactivated RNF43 or (an) activating R-Spondin fusion transcript(s). It is particularly preferred that these cancers are treated with LRP5/LRP6#1 as the LRP5/LRP6 antagonist and PD1-1 as the anti-PD-1 antibody. It is particularly preferred that these cancers are treated with LRP5/LRP6#5 as the LRP5/LRP6 antagonist and PD1-1 as the anti-PD-1 antibody. It is particularly preferred that these cancers are treated with LRP5/LRP6#6 as the LRP5/LRP6 antagonist and PD1-1 as the anti-PD-1 antibody. It is particularly preferred that these cancers are treated with LRP5/LRP6#1 as the LRP5/LRP6 antagonist and PD1-2 as the anti-PD-1 antibody. It is particularly preferred that these cancers are treated with LRP5/LRP6#5 as the LRP5/LRP6 antagonist and PD1-2 as the anti-PD-1 antibody. It is particularly preferred that these cancers are treated with LRP5/LRP6#6 as the LRP5/LRP6 antagonist and PD1-2 as the anti-PD-1 antibody. It is particularly preferred that these cancers are treated with LRP5/LRP6#1 as the LRP5/LRP6 antagonist and PD1-3 as the anti-PD-1 antibody. It is particularly preferred that these cancers are treated with LRP5/LRP6#5 as the LRP5/LRP6 antagonist and PD1-3 as the anti-PD-1 antibody. It is particularly preferred that these cancers are treated with LRP5/LRP6#6 as the LRP5/LRP6 antagonist and PD1-3 as the anti-PD-1 antibody.

In an alternative preferred embodiment, the combinations, compositions, kits, uses, methods and compounds for use according to the invention (including all embodiments) are used in the treatment of cancer patients suffering from any solid tumor that is refractory or resistant to checkpoint inhibitor therapies (e.g. to treatment with one or more immune checkpoint inhibitor and/or immuno modulators, e.g. one or more PD-1 antagonist(s) such as an anti-PD1 antibody. In one embodiment, said cancer is characterized in that it harbors a mutated/inactivated RNF43 or (an) activating R-Spondin fusion transcript(s). It is particularly preferred that these cancers are treated with LRP5/LRP6#1 as the LRP5/LRP6 antagonist and PD1-1 as the anti-PD-1 antibody. It is particularly preferred that these cancers are treated with LRP5/LRP6#5 as the LRP5/LRP6 antagonist and PD1-1 as the anti-PD-1 antibody. It is particularly preferred that these cancers are treated with LRP5/LRP6#6 as the LRP5/LRP6 antagonist and PD1-1 as the anti-PD-1 antibody. It is particularly preferred that these cancers are treated with LRP5/LRP6#1 as the LRP5/LRP6 antagonist and PD1-2 as the anti-PD-1 antibody. It is particularly preferred that these cancers are treated with LRP5/LRP6#5 as the LRP5/LRP6 antagonist and PD1-2 as the anti-PD-1 antibody. It is particularly preferred that these cancers are treated with LRP5/LRP6#6 as the LRP5/LRP6 antagonist and PD1-2 as the anti-PD-1 antibody. It is particularly preferred that these cancers are treated with LRP5/LRP6#1 as the LRP5/LRP6 antagonist and PD1-3 as the anti-PD-1 antibody. It is particularly preferred that these cancers are treated with LRP5/LRP6#5 as the LRP5/LRP6 antagonist and PD1-3 as the anti-PD-1 antibody. It is particularly preferred that these cancers are treated with LRP5/LRP6#6 as the LRP5/LRP6 antagonist and PD1-3 as the anti-PD-1 antibody. Examples for solid tumors are sufficiently known in the art. Similarly, the terms refractory or resistant are also known to the skilled person and are used herein in accordance with the definitions employed in the art.

Tumors which are refractory or resistant to checkpoint inhibitor therapies are also referred to herein as “immunotherapy-resistant tumors” or “immunotherapy-resistant non-T cell inflamed tumors”. It has recently been found that in the microenvironment of many tumors a high expression of specific immune cells can be found. This is referred to in the art “T cell-inflamed phenotype” and it has been observed that this phenotype correlates with said tumors being amenable to treatment with multiple immunotherapies including therapeutic vaccines and checkpoint blocking antibodies, such as anti-PD-1 antibodies. On the other hand, certain tumors lack this expression of immune cells in their microenvironment. These tumors are referred to in the art as “non-T cell inflamed tumors” and they were found to lack clinical benefit to immunotherapy, particularly with anti-PD-1 antibodies. In accordance with the present invention, the latter type of tumors with active Wnt signalling are a preferred target for the claimed combination therapy. It is particularly preferred that these cancers are treated with LRP5/LRP6#1 as the LRP5/LRP6 antagonist and PD1-1 as the anti-PD-1 antibody. It is particularly preferred that these cancers are treated with LRP5/LRP6#5 as the LRP5/LRP6 antagonist and PD1-1 as the anti-PD-1 antibody. It is particularly preferred that these cancers are treated with LRP5/LRP6#6 as the LRP5/LRP6 antagonist and PD1-1 as the anti-PD-1 antibody. It is particularly preferred that these cancers are treated with LRP5/LRP6#1 as the LRP5/LRP6 antagonist and PD1-2 as the anti-PD-1 antibody. It is particularly preferred that these cancers are treated with LRP5/LRP6#5 as the LRP5/LRP6 antagonist and PD1-2 as the anti-PD-1 antibody. It is particularly preferred that these cancers are treated with LRP5/LRP6#6 as the LRP5/LRP6 antagonist and PD1-2 as the anti-PD-1 antibody. It is particularly preferred that these cancers are treated with LRP5/LRP6#1 as the LRP5/LRP6 antagonist and PD1-3 as the anti-PD-1 antibody. It is particularly preferred that these cancers are treated with LRP5/LRP6#5 as the LRP5/LRP6 antagonist and PD1-3 as the anti-PD-1 antibody. It is particularly preferred that these cancers are treated with LRP5/LRP6#6 as the LRP5/LRP6 antagonist and PD1-3 as the anti-PD-1 antibody.

The present invention is not to be limited in scope by the specific embodiments described herein. Various modifications of the invention in addition to those described herein may become apparent to those skilled in the art from the present disclosure. Such modifications are intended to fall within the scope of the appended claims.

All patent applications cited herein are hereby incorporated by reference in their entireties.

### Example 1

Anti-Tumor Activity of the Exemplary LRP5/LRP6 in Combination with a Mouse Antibody to PD-1, in a Subcutaneous Syngeneic Mouse Model Derived from the Breast Cancer Cell Line EMT6 in Balb/c Mice

The efficacy of the exemplary LRP5/6 antagonist was tested in a s.c. cell line derived syngeneic model of mouse breast cancer (EMT6) as single agent and in combination with a mouse antibody to PD-1.

BALB/cJBomTac mice were used in this study. 1×10⁶ EMT6 breast cancer cells were injected per mouse to establish a tumor. Tumor volume was measured at least three times per week using a caliper. Treatment started when tumors had reached a median tumor volume of around 200 mm³ and was terminated after 30 days.

Ten tumor-bearing animals were treated with the exemplary LRP5/LRP6 intravenously (i.v.) twice a week and twice weekly i.p. with the exemplary mouse PD-1 antibody or a combination of both compounds. Ten animals were used in the vehicle/isotype control-treated group.

Animals were euthanized at the end of the study for ethical reasons based on the tumor mass (tumor≥1.5 cm³).

Cells

EMT6 cells were obtained from ATCC (catalog number ATCC® CRL2755™). A master cell bank (MCB) and a working cell bank (WCB) were established. Cells were cultured in T175 tissue culture flasks at 37° C. and 5% CO₂. The medium used was Waymouth's MB 752/1 supplemented with 15% fetal calf serum (HyClone® Fetal Bovine Serum Characterized; Cat No SH30071.03; by Thermo Scientific), and 2 mM L-Glutamine (L-Glutamine 200 mM (100×); Ref 25030-024; by Gibco by Life Technologies). Cultures were split every two-three days with a ratio of 1:10/1:15.

Mice

Mice were 7-8 week-old BALB/cJBomTac purchased from Taconic, Denmark. After arrival at the animal facility, mice were allowed to adjust to ambient conditions for at least 5 days before they were used for experiments. They were housed in Macrolon® type III cages in groups of ten under standardized conditions at 21.5±1.5° C. and 55±10% humidity. Standardized irradiated diet (PROVIMI KLIBA) and autoclaved tap water were provided ad libitum. Microchips implanted subcutaneously under isoflurane anesthesia were used to identify each mouse. Cage cards showing the study number, the animal number, the compound and dose level, the administration route as well as the schedule remained with the animals throughout the study.

Administration of Test Compounds

The LRP5LRP/6 antagonist was suspended in histidine buffer pH 6.5 and administered i.v. an application volume of 10 mL/kg per mouse twice weekly at 10 mg/kg dose for the first two weeks.

The PD-1 antibody was diluted in PBS and injected intraperitoneally with a volume of 10 mL/kg per mouse twice weekly at 10 mg/kg dose until the end of the study.

Monitoring Tumor Growth and Disease Progression The tumor diameter was measured three times a week (Monday, Wednesday and Friday) with a caliper. The volume of each tumor [in mm³] was calculated according to the formula “tumor volume=length*diameter2*π/6”. To monitor side effects of treatment, mice were inspected daily for abnormalities and body weight was determined daily. Animals were sacrificed at the end of the study. Animals with necrotic tumors or tumor sizes exceeding 1500 mm³ were sacrificed early during the studies for ethical reasons.

Results

Treatment of ETM6 tumors with the mouse antibody against PD-1 resulted in moderate tumor growth inhibition. Combination of the LRP5/LRP6 antagonist with the PD-1 antibody resulted in significantly increased efficacy when compared with single agent administrations, inducing tumor regressions in 4 out of 9 mice when compared to the single treatments when tumor regression was observed in only one out of 10 mice. The results demonstrating a synergistic effect of the combined administration compared to the single treatments are shown in FIG. 1. Increased survival, reported in Table 5 as the interval in days from start of treatment to the time when the tumor volume reached at least 500 mm³, was increased by the combination of the LRP5/LRP6 antagonist with the PD-1 antibody when compared to the single treatments.

Table 5 shows the anti-tumor activity of the exemplary LRP5/LRP6 antagonist as single agent and in combination with a mouse antibody to PD-1. The median refers to the interval (days) from start of treatment to the time when the tumor volume reached at least 500 mm³.

TABLE 5

| | Tumor LRP5/6 |
|---|---|
| Median (days) | Start of treatment: |
| Time to ≥500 mm³ | median tumor volume |
| tumor volume | 200 mm³ |
| Isotype | 9 |
| Anti-PD-1 | 19.5 |
| LRP antagonist | 12 |
| Combination | 27.5 |

Furthermore, histological analysis of the samples from mice showing tumor shrinkage (i.e. tumor volume at the end of the study is smaller when compared to the start of treatment) was performed. In particular, tumours were collected from all groups and fixed in 10% NBF (Formalin solution, neutral buffered, 10%) for FFPE (Formalin fixed paraffin embedded). Histomorphological analysis was performed on FFPE tumour tissues via hematoxylin-eosin (HE) staining for morphological assessment. No evidence of tumor at the end of the study on tissue from the site that formerly had a tumor, was reported only in the combination group (3 out of 9 mice), indicating that pathological complete response could be achieved only by the LRP5/6 antagonist combination with the PD-1 antibody treatment when compared to single treatment (Table 6).

Table 6 shows the anti-tumor activity of the exemplary LRP5/6 antagonist as single agent and in combination with a mouse antibody to PD-1. Complete response at the end of the study refers to no evidence remaining of cancer by histological examination on tissue from the site that formerly had a tumor, when compared to partial responses where tumor cells are detected.

TABLE 6

| Histological analysis of | Complete | Partial |
|---|---|---|
| responses (end of study) | responses | responses |
| Isotype | 0/9 | 1/9 |
| Anti-PD-1 | 0/10 | 1/10 |
| LRP5/6 antagonist | 0/10 | 0/10 |
| Combination | 3/9 | 1/9 |

### Example 2

Increased Tumor T Cell Infiltration of the Exemplary LRP5/LRP6 Antagonist in Combination with a Mouse Antibody to PD-1, in a Subcutaneous Syngeneic Mouse Model Derived from the Breast Cancer Cell Line EMT6 in Balb/c Mice

The ability of inducing T cell infiltration in tumors of an exemplary LRP5/LRP6 antagonist was tested in a s.c. cell line derived syngeneic model of mouse breast cancer (EMT6) as single agent and in combination with a mouse antibody to PD-1.

CD8 positive T cells were analysed in tumors at day 16 from mice treated with the single agent and in combination with a mouse antibody to PD-1, as reported in Example 1. Tumours were collected from all groups and fixed in 10% NBF for FFPE tissues, and immunohistochemistry (IHC) was performed with standard protocols using a rat monoclonal antibody to CD8a (53-6.7, eBioscience™, working dilution 1:200) to detect CD8 positive T cells. Quantitative assessment was performed using HALO™ Image Analysis Software and the level of significance was determined using the Graph Pad Prism software. An adjusted p value of less than 0.05 was considered to show a statistically significant difference between the groups. The results are shown in FIG. 2.

### Example 3

Effect of the Combination of LRP5/LRP6 Antagonist with an Anti-Human PD-1 Antibody in 3D Spheroids

To further assess the effect of a combination of an anti-LRP5/LRP6 antagonist (LRP5/LRP6#5 as defined above, also shown as SEQ ID NO:65) with an anti-human PD-1 antibody according to the invention (PD1-3 as defined in Table 3 above) on Wnt-driven immune suppression, an in vitro co-culture of tumor cells, activated human PBMCs and Wnt ligand (Wnt3a) was used and tumor cell viability was measured as readout.

To this end, tumor cells (NCI-H1437), stably transfected to express a red fluorescent protein (mKate2) and cultured in 3D as spheroids with activated human PBMCs and Wnt3a ligand (0.5 μg/ml) ligand, were treated with 1000 nM of the LRP5/LRP6 antagonist and 200 nM of the anti-PD-1 antibody, and cell viability was measured at indicated time points after compound addition.

3.1 Study Design

To establish an in vitro co-culture assay with tumor cells (NCI-H1437 non-small cell lung cancer cell line) and human PBMC, NCI-H1437 cells were stably transfected to express a red fluorescent protein (mKate2) and cultured in 3D as spheroids. To perform the co-culture assay, NCI-H1437mKate2 cells were seeded in a 96 well Spheroid Microplate (5000 cells per well). The NCI-H1437mKate2 cells were seeded in a volume of 200 μl of RPMI-1640+Glutamax medium (with 10% FCShi) per well. After 4 days spheroids had formed and 100 μl of Media was removed from each well and 100 μl of RPMI1640 medium+Glutamax (+10% FCShi) with or without 3×10⁵ PBMCs (activated for 72 hours with anti-CD3 and anti-CD28 antibodies (1 μg/ml)) were added to the appropriate wells.

Spheroids with and without PBMCs were exposed to either the anti-LRP5/LRP6 antagonist, Wnt3a, the anti-human PD-1 antibody or an isotype of the anti-human PD-1 antibody (as control), as monotherapy or in combinations. The compounds were only added once, at day 0 (4 days after tumor cell seeding in microplates).

12 hours after adding the compounds, the first measurement of the mKate2 fluorescence was taken and used to determine the cell viability of the tumor spheroids. This time point was used as the baseline (100%) to which the following measurements (taken in time intervals between 12 and 48 hours) were compared to. The fluorescence of mKate2 (Excitation: 590 nm; Emission 635 nm) was measured using the EnVision 2100 MULTILABEL READER (PerkinElmer). In the experiment, spheroids with PBMC and with or without treatment were run in six biological replicates until day two, five biological replicates at day 3 and 4, and four biological replicates at day 7 and 8.

Reagents and Tissue Culture Material
PBS (Gibco; 14190-094)
Trypsine EDTA (Gibco; 043-90317FU)
Ultra-LEAF™ Purified anti-human CD3 Antibody (Biolegend; 300332)
Ultra-LEAF™ Purified anti-human CD28 Antibody (Biolegend; 302934)
RPMI 1640+Glutamax (Gibco; 61870-010)
RPMI 1640 (Gibco; A10491-01)
FCS (HyClone; SH30084.03)
WNT3a (R&D 5036-WN/CF; Lot SVH181610A)
StemCell donor: B001000527; Lot: 1812180182

3.2 NCI-H1437mKate2 Culture

NCI-H1437mKate2 cell were cultured using RPMI 1640 (Gibco; A1 0491-01)+10% FCShi. The cells were split once a week (1:10) and medium was changed an additional time. For passaging, the cells were detached from the cell culture flask using Trypsin EDTA in PBS (Gibco; 043-90317FU): The medium was removed and 5 ml Trypsin was added for approximately 5 minutes at 37° C. Every minute, a visual check was performed to verify if the cells had already detached. After detachment, the cell/Trypsin solution was mixed with 45 ml of culture medium containing 10% FCShi, and centrifuged at 400×g for 5 min at room temperature. The cell pellet was re-suspended in an appropriate amount of medium and either counted for the co-culture assay or split 1:10 for cultivation. The cells were cultivated at 37° C. and 5% CO₂.

3.3 Thawing of PBMC and PBMC Activation

One vial with PBMCs (StemCell donor: B001000527; Lot: 1812180182) was thawed at RT until only a little piece of ice was left, then poured into 50 ml Falcon with 20 ml cold (2-8° C.) RPMI-1640+Glutamax. After vortexing, the Falcon tubes were centrifuged for 5 min at 400×g. Then the supernatant was discarded and the PBMC pellet was re-suspended in 1-2 ml assay medium (RPMI1640+Glutamax+10% FCShi).

The cells were counted and activated with anti-CD3 and anti-CD28 antibodies (1 μg/ml) for 72 hours (5×10\hat{ }6 cells/ml). After 72 hours the activated PBMC were centrifuged at 400×g for 5 minutes. The cell pellet was re-suspended in 1-2 ml of RPMI-1640+Glutamax medium (with 10% FCShi). Finally, cells were counted and diluted to 3×10\hat{ }6 cells/ml for the co-culture assay.

3.4 Spheroid Viability Change: Measurement and Analysis

The EnVision 2100 MULTILABEL READER (PerkinElmer) was used to determine cell viability changes of the NCI-H1437mKate2 Spheroids. The fluorescence of mKate2 was measured at Excitation 590 nm and Emission 635 nm and a measurement height of 4.1 mm. For analysis, the mean of the background (medium only) was subtracted from the measurements and the percent change of every well was calculated, comparing the new measurement of the well (minus background) with the baseline measurement (12 hours after adding the compounds and PBMCs). The standard deviation shown is the percentual standard deviation of percentual changes at the corresponding treatment and time point. The resulting percentual changes of the viability values were transferred to Graph Pad software and analysed by applying the 2way ANOVA in combination with the Bonferroni's multiple comparison test to determine statistical significance.

3.5 Statistical Analysis

The level of significance was determined using the Graph Pad Prism software. An (adjusted) p value of less than 0.05 for *, 0.01 for **, 0.001 for *** and <0,0001 for **** was considered to show a statistically significant difference between the groups.

3.6 Results

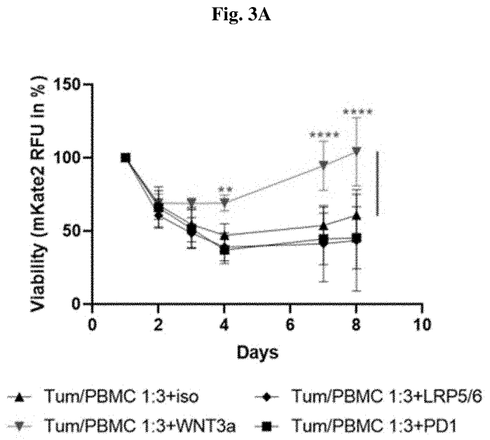

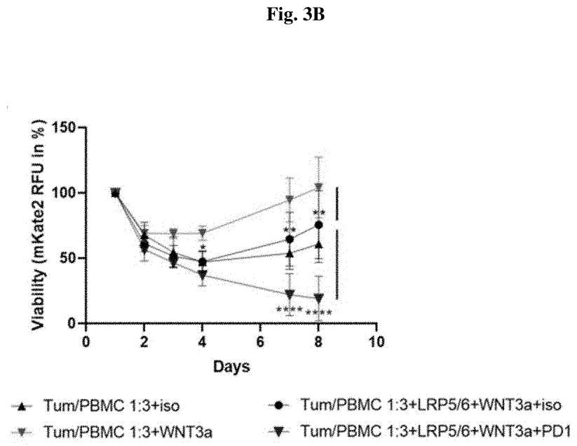

The effect of treatment with Wnt3a ligand, the LRP5/LRP6 antagonist or the anti-human PD-1 antibody on viability of tumor spheroids co-cultured with activated PMBCs is shown in FIG. 3A. Wnt3a treatment leads to a significant increase in tumor spheroids viability (inhibition of PBMC mediated tumor cell killing), detected at any time point between 4 and 8 days. Treatment with the LRP5/LRP6 antagonist or the anti-human PD-1 antibody has no significant effect on tumor spheroids viability, when compared to isotype treatment (control).

The effect of treatment with the LRP5/LRP6 antagonist as monotherapy or in combination with the anti-human PD-1 antibody in the presence of Wnt3a ligand is shown in FIG. 3B. Treatment with the LRP5/LRP6 antagonist as monotherapy suppresses the Wnt3a mediated increase in tumor spheroid viability (significant effect is reported between 4 and 8 days after start of treatment, Tum/PBMC 1:3+LRP5/6+WNT3a+iso vs. Tum/PBMC 1:3+iso). Therefore, treatment with the LRP5/LRP6 antagonist in the presence of Wnt3a ligand restores PBMC mediated inhibition of tumor spheroids viability.

Combination treatment of the LRP5/LRP6 antagonist and the anti-human PD-1 antibody leads to a significant decrease in tumor spheroid viability compared to the LRP5/LRP6 antagonist monotherapy (significant effect is reported between 7 and 8 days after start of treatment, Tum/PBMC 1:3+LRP5/6+WNT3a+PD1 vs. Tum/PBMC 1:3+LRP5/6+WNT3a+iso). Therefore, combination treatment of the LRP5/LRP6 antagonist and the anti-human PD-1 antibody leads to the enhancement of PBMC-mediated tumor cell killing, when compared to LRP5/LRP6 antagonist monotherapy.

3.7 Discussion

These results show that blockade of LRP5 and LRP6 in combination with a PD-1 antagonist results in PBMC-mediated killing of tumor spheroids. These data together with the data shown in Examples 1 and 2 indicate that a combination therapy according to the invention have a potent anti-tumour activity.

1. A method of treating and/or preventing a hyperproliferative disease, preferably cancer, comprising administering to a patient in need thereof a therapeutically effective amount of a polypeptide capable of specifically binding to LRP5 and LRP6 and a therapeutically effective amount of a PD-1 antibody, wherein the polypeptide capable of specifically binding to LRP5 and LRP6 is selected from the group consisting of
(i) a polypeptide comprising a first immunoglobulin single variable domain (ISVD) (a) comprising the following CDR sequences:
CDR1: TYTVG (=SEQ ID NO:40)
CDR2: AIRRRGSSTYYADSVKG (=SEQ ID NO:41)
CDR3: DTRTVALLQYRYDY (=SEQ ID NO:42), and
a second ISVD (b) comprising the following CDR sequences:
CDR1: SYAMG (=SEQ ID NO:49)
CDR2: AISWSGGSTYYADSVKG (=SEQ ID NO:50)
CDR3: SPIPYGSLLRRRNNYDY (=SEQ ID NO:51);
(ii) a polypeptide comprising a first ISVD (a) comprising the following CDR sequences:
CDR1: SYAMG (=SEQ ID NO:43)
CDR2: AIRRSGRRTYYADSVKG (=SEQ ID NO:44)
CDR3: ARRVRSSTRYNTGTWWWEY (=SEQ ID NO:45), and
a second ISVD (b) comprising the following CDR sequences:
CDR1: SYAMG (=SEQ ID NO:49)
CDR2: AISWSGGSTYYADSVKG (=SEQ ID NO:50)
CDR3: SPIPYGSLLRRRNNYDY (=SEQ ID NO:51);
(iii) a polypeptide comprising a first ISVD (a) comprising the following CDR sequences:
CDR1: RYTMG (=SEQ ID NO:46)
CDR2: AIVRSGGSTYYADSVKG (=SEQ ID NO:47)
CDR3: DRRGRGENYILLYSSGRYEY (=SEQ ID NO:48), and
a second ISVD (b) comprising the following CDR sequences:
CDR1: SYAMG (=SEQ ID NO:49)
CDR2: AISWSGGSTYYADSVKG (=SEQ ID NO:50)
CDR3: SPIPYGSLLRRRNNYDY (=SEQ ID NO:51);
(iv) a polypeptide comprising a first ISVD (a) comprising the following CDR sequences:
CDR1: TYTVG (=SEQ ID NO:40)
CDR2: AIRRRGSSTYYADSVKG (=SEQ ID NO:41)
CDR3: DTRTVALLQYRYDY (=SEQ ID NO:42), and
a second ISVD (b) comprising the following CDR sequences:
CDR1: SYAMG (=SEQ ID NO:52)
CDR2: AISWRSGSTYYADSVKG (=SEQ ID NO:53)
CDR3: DPRGYGVAYVSAYYEY (=SEQ ID NO:54);
(v) a polypeptide comprising a first ISVD (a) comprising the following CDR sequences:
CDR1: SYAMG (=SEQ ID NO:43)
CDR2: AIRRSGRRTYYADSVKG (=SEQ ID NO:44)
CDR3: ARRVRSSTRYNTGTWWWEY (=SEQ ID NO:45), and
a second ISVD (b) comprising the following CDR sequences:
CDR1: SYAMG (=SEQ ID NO:52)
CDR2: AISWRSGSTYYADSVKG (=SEQ ID NO:53)
CDR3: DPRGYGVAYVSAYYEY (=SEQ ID NO:54);
and
(vi) a polypeptide comprising a first ISVD (a) comprising the following CDR sequences:
CDR1: RYTMG (=SEQ ID NO:46)
CDR2: AIVRSGGSTYYADSVKG (=SEQ ID NO:47)
CDR3: DRRGRGENYILLYSSGRYEY (=SEQ ID NO:48), and
a second ISVD (b) comprising the following CDR sequences:
CDR1: SYAMG (=SEQ ID NO:52)
CDR2: AISWRSGSTYYADSVKG (=SEQ ID NO:53)
CDR3: DPRGYGVAYVSAYYEY (=SEQ ID NO:54);
and wherein the PD-1 antibody is selected from the group consisting of:
(i) an anti-PD1 antibody comprising heavy chain CDRs comprising the amino acid sequence of SEQ ID NO:1 (HCDR1), SEQ ID NO:2 (HCDR2) and SEQ ID NO:3 (HCDR3) and light chain CDRs comprising the amino acid sequence of SEQ ID NO:4 (LCDR1), SEQ ID NO:5 (LCDR2) and SEQ ID NO:6 (LCDR3);
(ii) an anti-PD1 antibody comprising heavy chain CDRs comprising the amino acid sequence of SEQ ID NO:7 (HCDR1), SEQ ID NO:8 (HCDR2) and SEQ ID NO:9 (HCDR3) and light chain CDRs comprising the amino acid sequence of SEQ ID NO:10 (LCDR1), SEQ ID NO:11 (LCDR2) and SEQ ID NO:12 (LCDR3);
and
(iii) an anti-PD1 antibody comprising heavy chain CDRs comprising the amino acid sequence of SEQ ID NO:13 (HCDR1), SEQ ID NO:14 (HCDR2) and SEQ ID NO:15 (HCDR3) and light chain CDRs comprising the amino acid sequence of SEQ ID NO:16 (LCDR1), SEQ ID NO:17 (LCDR2) and SEQ ID NO:18 (LCDR3).

2. A pharmaceutical composition comprising:
a polypeptide capable of specifically binding to LRP5 and LRP6; a PD-1 antibody; and, optionally, one or more pharmaceutically acceptable carriers, excipients and/or vehicles;
wherein the polypeptide capable of specifically binding to LRP5 and LRP6 is selected from the group consisting of
(i) a polypeptide comprising a first immunoglobulin single variable domain (ISVD) (a) comprising the following CDR sequences:
CDR1: TYTVG (=SEQ ID NO:40)
CDR2: AIRRRGSSTYYADSVKG (=SEQ ID NO:41)
CDR3: DTRTVALLQYRYDY (=SEQ ID NO:42), and
a second ISVD (b) comprising the following CDR sequences:
CDR1: SYAMG (=SEQ ID NO:49)
CDR2: AISWSGGSTYYADSVKG (=SEQ ID NO:50)
CDR3: SPIPYGSLLRRRNNYDY (=SEQ ID NO:51);
(ii) a polypeptide comprising a first ISVD (a) comprising the following CDR sequences:
CDR1: SYAMG (=SEQ ID NO:43)
CDR2: AIRRSGRRTYYADSVKG (=SEQ ID NO:44)
CDR3: ARRVRSSTRYNTGTWWWEY (=SEQ ID NO:45), and
a second ISVD (b) comprising the following CDR sequences:
CDR1: SYAMG (=SEQ ID NO:49)
CDR2: AISWSGGSTYYADSVKG (=SEQ ID NO:50)
CDR3: SPIPYGSLLRRRNNYDY (=SEQ ID NO:51);
(iii) a polypeptide comprising a first ISVD (a) comprising the following CDR sequences:
CDR1: RYTMG (=SEQ ID NO:46)
CDR2: AIVRSGGSTYYADSVKG (=SEQ ID NO:47)
CDR3: DRRGRGENYILLYSSGRYEY (=SEQ ID NO:48), and
a second ISVD (b) comprising the following CDR sequences:
CDR1: SYAMG (=SEQ ID NO:49)
CDR2: AISWSGGSTYYADSVKG (=SEQ ID NO:50)
CDR3: SPIPYGSLLRRRNNYDY (=SEQ ID NO:51);
(iv) a polypeptide comprising a first ISVD (a) comprising the following CDR sequences:
CDR1: TYTVG (=SEQ ID NO:40)
CDR2: AIRRRGSSTYYADSVKG (=SEQ ID NO:41)
CDR3: DTRTVALLQYRYDY (=SEQ ID NO:42), and
a second ISVD (b) comprising the following CDR sequences:
CDR1: SYAMG (=SEQ ID NO:52)
CDR2: AISWRSGSTYYADSVKG (=SEQ ID NO:53)
CDR3: DPRGYGVAYVSAYYEY (=SEQ ID NO:54);
(v) a polypeptide comprising a first ISVD (a) comprising the following CDR sequences:
CDR1: SYAMG (=SEQ ID NO:43)
CDR2: AIRRSGRRTYYADSVKG (=SEQ ID NO:44)
CDR3: ARRVRSSTRYNTGTWWWEY (=SEQ ID NO:45), and
a second ISVD (b) comprising the following CDR sequences:
CDR1: SYAMG (=SEQ ID NO:52)
CDR2: AISWRSGSTYYADSVKG (=SEQ ID NO:53)
CDR3: DPRGYGVAYVSAYYEY (=SEQ ID NO:54);
and
(vi) a polypeptide comprising a first ISVD (a) comprising the following CDR sequences:
CDR1: RYTMG (=SEQ ID NO:46)
CDR2: AIVRSGGSTYYADSVKG (=SEQ ID NO:47)
CDR3: DRRGRGENYILLYSSGRYEY (=SEQ ID NO:48), and
a second ISVD (b) comprising the following CDR sequences:
CDR1: SYAMG (=SEQ ID NO:52)
CDR2: AISWRSGSTYYADSVKG (=SEQ ID NO:53)
CDR3: DPRGYGVAYVSAYYEY (=SEQ ID NO:54);
and wherein the PD-1 antibody is selected from the group consisting of
(i) an anti-PD1 antibody comprising heavy chain CDRs comprising the amino acid sequence of SEQ ID NO:1 (HCDR1), SEQ ID NO:2 (HCDR2) and SEQ ID NO:3 (HCDR3) and light chain CDRs comprising the amino acid sequence of SEQ ID NO:4 (LCDR1), SEQ ID NO:5 (LCDR2) and SEQ ID NO:6 (LCDR3);
(ii) an anti-PD1 antibody comprising heavy chain CDRs comprising the amino acid sequence of SEQ ID NO:7 (HCDR1), SEQ ID NO:8 (HCDR2) and SEQ ID NO:9 (HCDR3) and light chain CDRs comprising the amino acid sequence of SEQ ID NO:10 (LCDR1), SEQ ID NO:11 (LCDR2) and SEQ ID NO:12 (LCDR3);
and
(iii) an anti-PD1 antibody comprising heavy chain CDRs comprising the amino acid sequence of SEQ ID NO:13 (HCDR1), SEQ ID NO:14 (HCDR2) and SEQ ID NO:15 (HCDR3) and light chain CDRs comprising the amino acid sequence of SEQ ID NO:16 (LCDR1), SEQ ID NO:17 (LCDR2) and SEQ ID NO:18 (LCDR3).

3. A kit comprising in one or more containers
a first pharmaceutical composition or dosage form comprising a polypeptide capable of specifically binding to LRP5 and LRP6 and, optionally, one or more pharmaceutically acceptable carriers, excipients and/or vehicles;
a second pharmaceutical composition or dosage form comprising a PD-1 antibody and, optionally, one or more pharmaceutically acceptable carriers, excipients and/or vehicles;
and optionally a package insert comprising printed instructions;
wherein the polypeptide capable of specifically binding to LRP5 and LRP6 is selected from the group consisting of
(i) a polypeptide comprising a first immunoglobulin single variable domain (ISVD) (a) comprising the following CDR sequences:
CDR1: TYTVG (=SEQ ID NO:40)
CDR2: AIRRRGSSTYYADSVKG (=SEQ ID NO:41)
CDR3: DTRTVALLQYRYDY (=SEQ ID NO:42), and
a second ISVD (b) comprising the following CDR sequences:
CDR1: SYAMG (=SEQ ID NO:49)
CDR2: AISWSGGSTYYADSVKG (=SEQ ID NO:50)
CDR3: SPIPYGSLLRRRNNYDY (=SEQ ID NO:51);
(ii) a polypeptide comprising a first ISVD (a) comprising the following CDR sequences:
CDR1: SYAMG (=SEQ ID NO:43)
CDR2: AIRRSGRRTYYADSVKG (=SEQ ID NO:44)
CDR3: ARRVRSSTRYNTGTWWWEY (=SEQ ID NO:45), and
a second ISVD (b) comprising the following CDR sequences:
CDR1: SYAMG (=SEQ ID NO:49)
CDR2: AISWSGGSTYYADSVKG (=SEQ ID NO:50)
CDR3: SPIPYGSLLRRRNNYDY (=SEQ ID NO:51);
(iii) a polypeptide comprising a first ISVD (a) comprising the following CDR sequences:
CDR1: RYTMG (=SEQ ID NO:46)
CDR2: AIVRSGGSTYYADSVKG (=SEQ ID NO:47)
CDR3: DRRGRGENYILLYSSGRYEY (=SEQ ID NO:48), and
a second ISVD (b) comprising the following CDR sequences:
CDR1: SYAMG (=SEQ ID NO:49)
CDR2: AISWSGGSTYYADSVKG (=SEQ ID NO:50)
CDR3: SPIPYGSLLRRRNNYDY (=SEQ ID NO:51);
(iv) a polypeptide comprising a first ISVD (a) comprising the following CDR sequences:
CDR1: TYTVG (=SEQ ID NO:40)
CDR2: AIRRRGSSTYYADSVKG (=SEQ ID NO:41)
CDR3: DTRTVALLQYRYDY (=SEQ ID NO:42), and
a second ISVD (b) comprising the following CDR sequences:
CDR1: SYAMG (=SEQ ID NO:52)
CDR2: AISWRSGSTYYADSVKG (=SEQ ID NO:53)
CDR3: DPRGYGVAYVSAYYEY (=SEQ ID NO:54);
(v) a polypeptide comprising a first ISVD (a) comprising the following CDR sequences:
CDR1: SYAMG (=SEQ ID NO:43)
CDR2: AIRRSGRRTYYADSVKG (=SEQ ID NO:44)
CDR3: ARRVRSSTRYNTGTWWWEY (=SEQ ID NO:45), and
a second ISVD (b) comprising the following CDR sequences:
CDR1: SYAMG (=SEQ ID NO:52)
CDR2: AISWRSGSTYYADSVKG (=SEQ ID NO:53)
CDR3: DPRGYGVAYVSAYYEY (=SEQ ID NO:54);
and
(vi) a polypeptide comprising a first ISVD (a) comprising the following CDR sequences:
CDR1: RYTMG (=SEQ ID NO:46)
CDR2: AIVRSGGSTYYADSVKG (=SEQ ID NO:47)
CDR3: DRRGRGENYILLYSSGRYEY (=SEQ ID NO:48), and
a second ISVD (b) comprising the following CDR sequences:
CDR1: SYAMG (=SEQ ID NO:52)
CDR2: AISWRSGSTYYADSVKG (=SEQ ID NO:53)
CDR3: DPRGYGVAYVSAYYEY (=SEQ ID NO:54);
and wherein the PD-1 antibody is selected from the group consisting of
(i) an anti-PD1 antibody comprising heavy chain CDRs comprising the amino acid sequence of SEQ ID NO:1 (HCDR1), SEQ ID NO:2 (HCDR2) and SEQ ID NO:3 (HCDR3) and light chain CDRs comprising the amino acid sequence of SEQ ID NO:4 (LCDR1), SEQ ID NO:5 (LCDR2) and SEQ ID NO:6 (LCDR3);
(ii) an anti-PD1 antibody comprising heavy chain CDRs comprising the amino acid sequence of SEQ ID NO:7 (HCDR1), SEQ ID NO:8 (HCDR2) and SEQ ID NO:9 (HCDR3) and light chain CDRs comprising the amino acid sequence of SEQ ID NO:10 (LCDR1), SEQ ID NO:11 (LCDR2) and SEQ ID NO:12 (LCDR3),
and
(iii) an anti-PD1 antibody comprising heavy chain CDRs comprising the amino acid sequence of SEQ ID NO:13 (HCDR1), SEQ ID NO:14 (HCDR2) and SEQ ID NO:15 (HCDR3) and light chain CDRs comprising the amino acid sequence of SEQ ID NO:16 (LCDR1), SEQ ID NO:17 (LCDR2) and SEQ ID NO:18 (LCDR3).

4. The method of treatment according to claim 1,
wherein the polypeptide capable of specifically binding to LRP5 and LRP6 is selected from the group consisting of
(i) a polypeptide comprising a first ISVD comprising an amino acid sequence of SEQ ID NO:58, and a second ISVD comprising the sequence of SEQ ID NO:61;
(ii) a polypeptide comprising a first ISVG comprising an amino acid sequence of SEQ ID NO:59 and a second ISVD comprising the sequence of SEQ ID NO:61;
(iii) a polypeptide comprising a first ISVD comprising the sequence of SEQ ID NO:60, and a second ISVD comprising the sequence of SEQ ID NO:61;
(iv) a polypeptide comprising a first ISVD comprising an amino acid sequence of SEQ ID NO:58 and a second ISVD comprising the sequence of SEQ ID NO:62;
(v) a polypeptide comprising a first ISVD comprising an amino acid sequence of SEQ ID NO:59 and a second ISVD comprising the sequence of SEQ ID NO:62;
and
(vi) a polypeptide comprising a first ISVD comprising an amino acid sequence of SEQ ID NO:60 and a second ISVD comprising the sequence of SEQ ID NO:62;
preferably wherein the polypeptide capable of specifically binding to LRP5 and LRP6 further comprises an Alb11 domain comprising the amino acid sequence of SEQ ID NO:63.

5. The method of treatment according to claim 1,
wherein the polypeptide capable of specifically binding to LRP5 and LRP6 comprises a polypeptide comprising an amino acid sequence selected from the group consisting of SEQ ID NO: 64, SEQ ID NO:65 and SEQ ID NO:66.

6. The method of treatment according to claim 1,
wherein the anti-PD1 antibody is selected from the group consisting of
(i) an antibody having a heavy chain variable domain comprising the amino acid sequence of SEQ ID NO:19 and a light chain variable domain comprising the amino acid sequence of SEQ ID NO:20;
(ii) an antibody having a heavy chain variable domain comprising the amino acid sequence of SEQ ID NO:21 and a light chain variable domain comprising the amino acid sequence of SEQ ID NO:22;
(iii) an antibody having a heavy chain variable domain comprising the amino acid sequence of SEQ ID NO:23 and a light chain variable domain comprising the amino acid sequence of SEQ ID NO:24;
(iv) an antibody having a heavy chain variable domain comprising the amino acid sequence of SEQ ID NO:25 and a light chain variable domain comprising the amino acid sequence of SEQ ID NO:26;
and
(v) an antibody having a heavy chain variable domain comprising the amino acid sequence of SEQ ID NO:27 and a light chain variable domain comprising the amino acid sequence of SEQ ID NO:28.

7. The method of treatment according to claim 1,
wherein the PD-1 antibody is selected from the group consisting of
(i) an antibody having a heavy chain comprising the amino acid sequence of SEQ ID NO: 29 and a light chain comprising the amino acid sequence of SEQ ID NO: 30;
(ii) an antibody having a heavy chain comprising the amino acid sequence of SEQ ID NO: 31 and a light chain comprising the amino acid sequence of SEQ ID NO: 32;
(iii) an antibody having a heavy chain comprising the amino acid sequence of SEQ ID NO: 33 and a light chain comprising the amino acid sequence of SEQ ID NO: 34;
(iv) an antibody having a heavy chain comprising the amino acid sequence of SEQ ID NO: 35 and a light chain comprising the amino acid sequence of SEQ ID NO: 36;
and
(v) an antibody having a heavy chain comprising the amino acid sequence of SEQ ID NO: 37 and a light chain comprising the amino acid sequence of SEQ ID NO: 38.

8. The method of treatment according to claim 1,
wherein the PD-1 antibody is to be administered simultaneously, concurrently, sequentially, successively, alternately or separately with the polypeptide capable of specifically binding to LRP5 and LRP6.

9. The method of treatment according to claim 1,
wherein the polypeptide capable of specifically binding to LRP5 and LRP6 and the PD-1 antibody are to be administered according to the following treatment regimen:
(i) a first treatment period, wherein the polypeptide capable of specifically binding to LRP5 and LRP6 and the PD-1 antibody are to be administered simultaneously or concurrently, preferably every three or four weeks;
and
(ii) a second treatment period, wherein only the PD-1 antibody is to be administered and the polypeptide capable of specifically binding to LRP5 and LRP6 is not to be administered, preferably wherein the PD-1 antibody is to be administered every three or four weeks.

10. The method of treatment according to claim 9,
wherein the first treatment period is 3 or 6 weeks, when the polypeptide capable of specifically binding to LRP5 and LRP6 and PD-1 antibody are administered every three weeks; or
the first treatment period is 4 or 8 weeks when the polypeptide capable of specifically binding to LRP5 and LRP6 and PD-1 antibody are administered every four weeks.

11. The method of treatment according to claim 9, wherein the administration is an intravenous administration.

12. The method of treatment according to claim 1,
wherein the hyperproliferative disease to be treated is a cancer selected from the group consisting of gastrointestinal cancers, melanoma tumours, bladder cancer and lung cancer (e.g. NSCLC).

13. The method of treatment according to claim 12,
wherein the gastrointestinal cancer is esophageal cancer (e.g., gastroesophageal junction cancer), stomach (gastric) cancer, hepatocellular carcinoma, biliary tract cancer (e.g., cholangiocarcinoma), gallbladder cancer, pancreatic cancer or colorectal cancer (CRC).

14. The method of treatment according to claim 12,
wherein the cancer is an immunotherapy-resistant tumour.

15. The method of treatment according to claim 1,
wherein the hyperproliferative disease to be treated is a solid immunotherapy-resistant tumour.


SEQUENCE LISTING (66 sequences)

SEQ ID NO: 1 - protein, 10 aa, Artificial Sequence
  PD1-1HCDR1
GFTFSASAMS

SEQ ID NO: 2 - protein, 17 aa, Artificial Sequence
  PD1-1HCDR2
YISGGGGDTYYSSSVKG

SEQ ID NO: 3 - protein, 11 aa, Artificial Sequence
  PD1-1HCDR3
HSNVNYYAMDY

SEQ ID NO: 4 - protein, 15 aa, Artificial Sequence
  PD1-1LCDR1
RASENIDTSGISFMN

SEQ ID NO: 5 - protein, 7 aa, Artificial Sequence
  PD1-1LCDR2
VASNQGS

SEQ ID NO: 6 - protein, 9 aa, Artificial Sequence
  PD1-1LCDR3
QQSKEVPWT

SEQ ID NO: 7 - protein, 10 aa, Artificial Sequence
  PD1-2HCDR1
GFTFSASAMS

SEQ ID NO: 8 - protein, 17 aa, Artificial Sequence
  PD1-2HCDR2
YISGGGGDTYYSSSVKG

SEQ ID NO: 9 - protein, 11 aa, Artificial Sequence
  PD1-2HCDR3
HSNPNYYAMDY

SEQ ID NO: 10 - protein, 15 aa, Artificial Sequence
  PD1-2LCDR1
RASENIDTSGISFMN

SEQ ID NO: 11 - protein, 7 aa, Artificial Sequence
  PD1-2LCDR2
VASNQGS

SEQ ID NO: 12 - protein, 9 aa, Artificial Sequence
  PD1-2LCDR3
QQSKEVPWT

SEQ ID NO: 13 - protein, 10 aa, Artificial Sequence
  PD1-3HCDR1
GFTFSKSAMS

SEQ ID NO: 14 - protein, 17 aa, Artificial Sequence
  PD1-3HCDR2
YISGGGGDTYYSSSVKG

SEQ ID NO: 15 - protein, 11 aa, Artificial Sequence
  PD1-3HCDR3
HSNVNYYAMDY

SEQ ID NO: 16 - protein, 15 aa, Artificial Sequence
  PD1-3LCDR1
RASENIDVSGISFMN

SEQ ID NO: 17 - protein, 7 aa, Artificial Sequence
  PD1-3LCDR2
VASNQGS

SEQ ID NO: 18 - protein, 9 aa, Artificial Sequence
  PD1-3LCDR3
QQSKEVPWT

SEQ ID NO: 19 - protein, 120 aa, Artificial Sequence
  PD1VH1
EVMLVESGGGLVQPGGSLRLSCTASGFTFSASAMSWVRQAPGKGLEWVAYISGGGGDTYYSSSVKGRFTISRDNAKNSLYLQMNSLRAEDTAVYYCARHSNVNYYAMDYWGQGTLVTVSS

SEQ ID NO: 20 - protein, 111 aa, Artificial Sequence
  PD1VL1
EIVLTQSPATLSLSPGERATMSCRASENIDTSGISFMNWYQQKPGQAPKLLIYVASNQGSGIPARFSGSGSGTDFTLTISRLEPEDFAVYYCQQSKEVPWTFGQGTKLEIK

SEQ ID NO: 21 - protein, 120 aa, Artificial Sequence
  PD1VH2
EVMLVESGGGLVQPGGSLRLSCTASGFTFSASAMSWVRQAPGKGLEWVAYISGGGGDTYYSSSVKGRFTISRDNAKNSLYLQMNSLRAEDTAVYYCARHSNPNYYAMDYWGQGTLVTVSS

SEQ ID NO: 22 - protein, 111 aa, Artificial Sequence; identical to SEQ ID NO: 20
  PD1VL2

SEQ ID NO: 23 - protein, 120 aa, Artificial Sequence
  PD1VH3
EVMLVESGGGLVQPGGSLRLSCTASGFTFSKSAMSWVRQAPGKGLEWVAYISGGGGDTYYSSSVKGRFTISRDNAKNSLYLQMNSLRAEDTAVYYCARHSNVNYYAMDYWGQGTLVTVSS

SEQ ID NO: 24 - protein, 111 aa, Artificial Sequence
  PD1VL3
EIVLTQSPATLSLSPGERATMSCRASENIDVSGISFMNWYQQKPGQAPKLLIYVASNQGSGIPARFSGSGSGTDFTLTISRLEPEDFAVYYCQQSKEVPWTFGQGTKLEIK

SEQ ID NO: 25 - protein, 120 aa, Artificial Sequence; identical to SEQ ID NO: 23
  PD1VH4

SEQ ID NO: 26 - protein, 111 aa, Artificial Sequence; identical to SEQ ID NO: 24
  PD1VL4

SEQ ID NO: 27 - protein, 120 aa, Artificial Sequence; identical to SEQ ID NO: 23
  PD1VH5

SEQ ID NO: 28 - protein, 111 aa, Artificial Sequence; identical to SEQ ID NO: 24
  PD1VL5

SEQ ID NO: 29 - protein, 446 aa, Artificial Sequence
  PD1HC1
EVMLVESGGGLVQPGGSLRLSCTASGFTFSASAMSWVRQAPGKGLEWVAYISGGGGDTYYSSSVKGRFTISRDNAKNSLYLQMNSLRAEDTAVYYCARHSNVNYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 30 - protein, 218 aa, Artificial Sequence
  PD1LC1
EIVLTQSPATLSLSPGERATMSCRASENIDTSGISFMNWYQQKPGQAPKLLIYVASNQGSGIPARFSGSGSGTDFTLTISRLEPEDFAVYYCQQSKEVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 446 aa, Artificial Sequence
  PD1HC2
EVMLVESGGGLVQPGGSLRLSCTASGFTFSASAMSWVRQAPGKGLEWVAYISGGGGDTYYSSSVKGRFTISRDNAKNSLYLQMNSLRAEDTAVYYCARHSNPNYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 32 - protein, 218 aa, Artificial Sequence; identical to SEQ ID NO: 30
  PD1LC2

SEQ ID NO: 33 - protein, 446 aa, Artificial Sequence
  PD1HC3
EVMLVESGGGLVQPGGSLRLSCTASGFTFSKSAMSWVRQAPGKGLEWVAYISGGGGDTYYSSSVKGRFTISRDNAKNSLYLQMNSLRAEDTAVYYCARHSNVNYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 34 - protein, 218 aa, Artificial Sequence
  PD1LC3
EIVLTQSPATLSLSPGERATMSCRASENIDVSGISFMNWYQQKPGQAPKLLIYVASNQGSGIPARFSGSGSGTDFTLTISRLEPEDFAVYYCQQSKEVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 446 aa, Artificial Sequence; identical to SEQ ID NO: 33
  PD1HC4

SEQ ID NO: 36 - protein, 218 aa, Artificial Sequence; identical to SEQ ID NO: 34
  PD1LC4

SEQ ID NO: 37 - protein, 446 aa, Artificial Sequence; identical to SEQ ID NO: 33
  PD1HC5

SEQ ID NO: 38 - protein, 218 aa, Artificial Sequence; identical to SEQ ID NO: 34
  PD1LC5

SEQ ID NO: 39 - protein, 5 aa, Artificial Sequence
  motif for B7-1 and B7-2 binding
MYPPY

SEQ ID NO: 40 - protein, 5 aa, Artificial Sequence
  Wnt1-333E06mod-CDR1
TYTVG

SEQ ID NO: 41 - protein, 17 aa, Artificial Sequence
  Wnt1-333E06mod-CDR2
AIRRRGSSTYYADSVKG

SEQ ID NO: 42 - protein, 14 aa, Artificial Sequence
  Wnt1-333E06mod-CDR3
DTRTVALLQYRYDY

SEQ ID NO: 43 - protein, 5 aa, Artificial Sequence
  Wnt1-333G06-CDR1
SYAMG

SEQ ID NO: 44 - protein, 17 aa, Artificial Sequence
  Wnt1-333G06-CDR2
AIRRSGRRTYYADSVKG

SEQ ID NO: 45 - protein, 19 aa, Artificial Sequence
  Wnt1-333G06-CDR3
ARRVRSSTRYNTGTWWWEY

SEQ ID NO: 46 - protein, 5 aa, Artificial Sequence
  Wnt1-332D03mod-CDR1
RYTMG

SEQ ID NO: 47 - protein, 17 aa, Artificial Sequence
  Wnt1-332D03mod-CDR2
AIVRSGGSTYYADSVKG

SEQ ID NO: 48 - protein, 20 aa, Artificial Sequence
  Wnt1-332D03mod-CDR3
DRRGRGENYILLYSSGRYEY

SEQ ID NO: 49 - protein, 5 aa, Artificial Sequence
  Wnt3a-093A01-CDR1
SYAMG

SEQ ID NO: 50 - protein, 17 aa, Artificial Sequence
  Wnt3a-093A01-CDR2
AISWSGGSTYYADSVKG

SEQ ID NO: 51 - protein, 17 aa, Artificial Sequence
  Wnt3a-093A01-CDR3
SPIPYGSLLRRRNNYDY

SEQ ID NO: 52 - protein, 5 aa, Artificial Sequence
  Wnt3a-367B10-CDR1
SYAMG

SEQ ID NO: 53 - protein, 17 aa, Artificial Sequence
  Wnt3a-367B10-CDR2
AISWRSGSTYYADSVKG

SEQ ID NO: 54 - protein, 16 aa, Artificial Sequence
  Wnt3a-367B10-CDR3
DPRGYGVAYVSAYYEY

SEQ ID NO: 55 - protein, 5 aa, Artificial Sequence
  Alb11 domain CDR1
SFGMS

SEQ ID NO: 56 - protein, 17 aa, Artificial Sequence
  Alb11 domain CDR2
SISGSGSDTLYADSVKG

SEQ ID NO: 57 - protein, 6 aa, Artificial Sequence
  Alb11 domain CDR3
GGSLSR

SEQ ID NO: 58 - protein, 123 aa, Artificial Sequence
  Wnt1-333E06mod VHH
AVQLVESGGGLVQPGGSLRLSCAASGRTFSTYTVGWFRQAPGKEREFVAAIRRRGSSTYYADSVKGRFTISRDNSKNTVYLQMNSLRPEDTAVYYCAADTRTVALLQYRYDYWGQGTLVTVSS

SEQ ID NO: 59 - protein, 128 aa, Artificial Sequence
  Wnt1-333G06 VHH
AVQLVESGGGLVQPGGSLRLSCAASGGTFSSYAMGWFRQAPGKEREFVAAIRRSGRRTYYADSVKGRFTISRDNSKNTVYLQMNSLRPEDTAVYYCAAARRVRSSTRYNTGTWWWEYWGQGTLVTVSS

SEQ ID NO: 60 - protein, 129 aa, Artificial Sequence
  Wnt1-332D03mod VHH
AVQLVESGGGLVQPGGSLRLSCAASGLTFSRYTMGWFRQAPGKEREFVAAIVRSGGSTYYADSVKGRFTISRDNSKNTVYLQMNSLRPEDTAVYYCAADRRGRGENYILLYSSGRYEYWGQGTLVTVSS

SEQ ID NO: 61 - protein, 126 aa, Artificial Sequence
  Wnt3a-093A01 VHH
EVQLVESGGGLVQPGGSLRLSCAASGRTFSSYAMGWFRQAPGKEREFVAAISWSGGSTYYADSVKGRFTISRDNSKNTVYLQMNSLRPEDTAVYYCAASPIPYGSLLRRRNNYDYWGQGTLVTVSS

SEQ ID NO: 62 - protein, 125 aa, Artificial Sequence
  Wnt3a-367B10 VHH
EVQLVESGGGLVQPGGSLRLSCAASGGTFSSYAMGWFRQAPGKEREFVAAISWRSGSTYYADSVKGRFTISRDNSKNTVYLQMNSLRPEDTAVYYCAADPRGYGVAYVSAYYEYWGQGTLVTVSS

SEQ ID NO: 63 - protein, 115 aa, Artificial Sequence [sequence omitted]

SEQ ID NO: 64 - protein, 435 aa, Artificial Sequence
  Polypeptide capable of specifically binding to LRP5 and LRP6 (1)
AVQLVESGGGLVQPGGSLRLSCAASGRTFSTYTVGWFRQAPGKEREFVAAIRRRGSSTYYADSVKGRFTISRDNSKNTVYLQMNSLRPEDTAVYYCAADTRTVALLQYRYDYWGQGTLVTVSSGGGGSGGGGSGGGGSGGGGSGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGGSGGGGSGGGGSGGGGSGGGGSGGGGSEVQLVESGGGLVQPGGSLRLSCAASGRTFSSYAMGWFRQAPGKEREFVAAISWSGGSTYYADSVKGRFTISRDNSKNTVYLQMNSLRPEDTAVYYCAASPIPYGSLLRRRNNYDYWGQGTLVTVSSA

SEQ ID NO: 65 - protein, 439 aa, Artificial Sequence
  Polypeptide capable of specifically binding to LRP5 and LRP6 (2)
AVQLVESGGGLVQPGGSLRLSCAASGGTFSSYAMGWFRQAPGKEREFVAAIRRSGRRTYYADSVKGRFTISRDNSKNTVYLQMNSLRPEDTAVYYCAAARRVRSSTRYNTGTWWWEYWGQGTLVTVSSGGGGSGGGGSGGGGSGGGGSGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGGSGGGGSGGGGSGGGGSGGGGSGGGGSEVQLVESGGGLVQPGGSLRLSCAASGGTFSSYAMGWFRQAPGKEREFVAAISWRSGSTYYADSVKGRFTISRDNSKNTVYLQMNSLRPEDTAVYYCAADPRGYGVAYVSAYYEYWGQGTLVTVSSA

SEQ ID NO: 66 - protein, 440 aa, Artificial Sequence
  Polypeptide capable of specifically binding to LRP5 and LRP6 (3)
AVQLVESGGGLVQPGGSLRLSCAASGLTFSRYTMGWFRQAPGKEREFVAAIVRSGGSTYYADSVKGRFTISRDNSKNTVYLQMNSLRPEDTAVYYCAADRRGRGENYILLYSSGRYEYWGQGTLVTVSSGGGGSGGGGSGGGGSGGGGSGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGGSGGGGSGGGGSGGGGSGGGGSGGGGSEVQLVESGGGLVQPGGSLRLSCAASGGTFSSYAMGWFRQAPGKEREFVAAISWRSGSTYYADSVKGRFTISRDNSKNTVYLQMNSLRPEDTAVYYCAADPRGYGVAYVSAYYEYWGQGTLVTVSSA